(19) United States
(12) Patent Application Publication
(10) Pub. No.: US 2024/0327541 A1
(43) Pub. Date: Oct. 3, 2024

(54) METHODS AND COMPOSITIONS COMPRISING NOVEL BISPECIFIC ANTIBODIES

(71) Applicant: SYNOA THERAPEUTICS, LLC, Philadelphia, PA (US)

(72) Inventor: Jonathan Larkin, Philadelphia, PA (US)

(73) Assignee: SYNOA THERAPEUTICS, LLC, Philadelphia, PA (US)

(21) Appl. No.: 18/737,179

(22) Filed: Jun. 7, 2024

Related U.S. Application Data
(63) Continuation of application No. 18/463,037, filed on Sep. 7, 2023, now Pat. No. 12,043,672.
(60) Provisional application No. 63/404,273, filed on Sep. 7, 2022.

(51) Int. Cl. C07K 16/40; C07K 16/22

(52) U.S. Cl. CPC C07K 16/40; C07K 16/22; C07K 2317/21; C07K 2317/24; C07K 2317/31; C07K 2317/76; C07K 2317/92

(57) ABSTRACT

Provided herein are novel bispecific antibodies for the treatment of chronic pain. The bispecific antibodies comprise two or more antigen binding components, wherein at least one antigen binding component is capable of binding to human ADAMTS5, and wherein at least one antigen binding component is capable of binding to nerve growth factor (NGF).

### PRIORITY CLAIM AND CROSS-REFERENCE

This application is a continuation of U.S. patent application Ser. No. 18/463,037, filed on Sep. 7, 2023, which claims priority under 35 U.S.C. § 119 (e) to U.S. Patent Application No. 63/404,273 filed on Sep. 7, 2022. The foregoing application, and all documents cited therein, together with any manufacturer's instructions, descriptions, mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### TECHNICAL FIELD

Embodiments of this disclosure relate generally to novel non-opioid therapeutics for the treatment of chronic pain, such as osteoarthritis, comprising the use of antibodies that address both pain and structural cartilage degeneration. Provided herein are novel bispecific antibodies, pharmaceutical compositions comprising the same, and methods for making and using the same.

### SEQUENCE LISTING

The instant application contains a Sequence Listing which has been submitted electronically and is hereby incorporated by reference in its entirety. Said XML copy, was created Sep. 7, 2023, is named H6487-0003.xml and is 215,545 bytes in size.

### BACKGROUND OF THE INVENTION

Chronic pain is a major public health problem. It is estimated to affect more than 100 million people in the United States and about 20-30% of the population worldwide. The prevalence of persistent pain is expected to rise in the near future as the incidence of associated diseases (including diabetes, obesity, cardiovascular disorders, arthritis, and cancer) increases in the aging U.S. population.

According to the Merck Manual, chronic pain may be the result of a variety of causes. In some instances, chronic pain can happen because of a long-term disease or an injury that fails to heal. Oftentimes, chronic pain can be caused by an ongoing problem such as a long-lasting disorder such as arthritis, diabetes, cancer, or fibromyalgia. Sometimes the nervous system becomes more sensitive than usual to pain signals resulting in chronic pain and often causing long-term nerve signaling disruption. Chronic pain can also lead to other symptoms such as feeling tired, problems sleeping, not feeling hungry, or not being interested in sex. Chronic pain can make it hard to work and do normal daily activities. Also associated with chronic pain are emotional symptoms, such as depression, anxiety, or withdrawing from social activities.

One of the most common causes of chronic pain is arthritis. Arthritis is not a single disease; the term refers to joint pain or joint disease, and there are more than 100 types of arthritis and related conditions. People of all ages, races and sexes live with arthritis, and it is the leading cause of disability in the U.S. It is most common among women, and although it is not a disease of aging, some types of arthritis occur in older people more than younger people. Common arthritis symptoms include swelling, pain, stiffness and diminished range of motion in joints. Symptoms vary from mild to severe and may come and go. Some may stay about the same for years, but symptoms can also progress and get worse over time. Severe arthritis can result in chronic pain, difficulty performing daily activities and make walking and climbing stairs painful and grueling.

Arthritis can also cause permanent joint changes. These may be visible, such as knobby finger joints, but often the damage can be seen only on X-rays. Some types of arthritis affect the heart, eyes, lungs, kidneys and skin as well as the joints.

Osteoarthritis (OA) is by far the most common type of arthritis. It can damage almost any joint but mainly occurs in the hands, spine, hips and knees. OA was once considered a wear-and-tear disease in which cartilage, the protective layer on the ends of bones, wore down after years of use. But with further research, the thinking about OA has changed. Doctors now know that OA is a disease of the whole joint, not just cartilage (although cartilage remains a primary focus). Bones in affected joints become weaker, the connective tissue that holds the joint together deteriorates and inflammation damages the joint lining. Contrary to decades of belief, inflammation plays a key role in OA, just as it does in most other types of arthritis.

Doctors generally threat chronic pain with medicines, physical therapy, occupational therapy as well as relaxation techniques, hypnosis, biofeedback, and other behavioral and psychological therapies including treatments for emotional symptoms.

Depending upon the severity of disease, pain may be managed with NSAIDs (non-steroidal anti-inflammatory drugs) such as over-the-counter pain medicines, for example, aspirin, ibuprofen or acetaminophen. Also used are antidepressants, corticosteroids, muscle relaxers, topical products, sedatives or medical marijuana.

In certain severe cases, opioids may be prescribed to help manage pain. Opioids are powerful analgesics which are commonly used and found to be effective for many types of pain. However, opioids can produce significant side effects, including constipation, nausea, mental clouding, and respiratory depression, which can sometimes lead to death. Additionally, opioids often don't work for the long term. Although opioids can be successfully used to treat moderate to severe pain ranging from cancer to broken bones, there is a growing epidemic of opioid addiction and abuse. Long-term opioid use can result in physical dependence, making it difficult to discontinue use even when the original cause of pain is no longer present. Furthermore, there is mounting evidence that long-term opioid use for pain can actually produce a chronic pain state, whereby patients find themselves in a vicious cycle, where opioids are used to treat pain caused by previous opioid use. Data from the Centers for Disease Control and Prevention indicate that the prescribing of opioids by clinicians has increased threefold in the last 20 years, contributing to the problem of prescription opioid abuse. Today, the number of people who die from prescription opioids exceeds the number of those who die from heroin and cocaine, combined.

Negative side-effects and complications related to therapeutic intervention are not restricted to opioids. Indeed, every medication has a potential for side effects, some happen to be more serious than others. Complications from medical treatments for chronic pain can include: acute liver failure from acetaminophen treatment, addiction and/or overdose, mood changes, confusion and respiratory issues from nerve pain medications, spinal cord damage or infection from spinal cord stimulators.

A further issue with currently available drugs and medicines, is that they are focused on treating one aspect of chronic pain, namely the pain component only. There are no medications currently available that are effective for treating pain, and for treating the underlying causes of pain, such as for example, cartilage degeneration. With issues and complications such as addiction, toxicity and modest long term efficacy, currently available pain treatments are inadequate to meet the needs of a majority of patients suffering from pain, chronic pain, osteoarthritis, and arthritis.

What is needed are non-opioid compositions and methods for treating chronic pain, including osteoarthritis. Such compositions and methods should be effective in treating pain and also address the underlying case of pain. What is also needed are single molecule therapeutics having dual function wherein such therapeutics are effective, non-toxic and non-addictive.

### SUMMARY OF THE INVENTION

In an embodiment, the present disclosure relates to novel compositions and methods for treating chronic pain, wherein such novel methods and compositions simultaneously inhibit both pain and structural tissue degeneration. In certain embodiments, the compositions comprise a single therapeutic molecule, and in certain embodiments such therapeutic molecule may provide extended relief.

### BRIEF DESCRIPTION OF FIGURES

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 provides schematic representations of certain bispecific and monoclonal control antibody constructs.

FIG. 2 provides a schematic showing the design of binding and affinity kinetics experiments performed using a Bio-Layer Intreferometry (BLI) technology platform (Octet RH16, Sartorius). Bispecific and monoclonal control antibodies were measured for binding to the target human proteins (ADAMTS5 and NGF) in sequential order and results were used to quantify binding and affinity to enable characterization and rank ordering (FIG. 2).

FIG. 3A provides a tabular and FIG. 3B provides a graphical visualization of experimental results demonstrating quantitative target binding and affinity kinetics of bispecific and control antibodies. Experimental data was analyzed for quantitative antibody loading and target binding (measured as nm shift) and affinity kinetics (association [Kon], dissociation [Kdis] and equilibrium dissociation [KD] constants) using Octet Data Analysis Software.

FIG. 4 provides a table summarizing affinities for bispecific and monoclonal antibodies as assessed during the second association/dissociation step from sequential binding experiments.

FIG. 5A and FIG. 5B provide graphs demonstrating comparative quantitation of different bispecific antibody capacities to bind both targets in relation to monoclonal and isotype control antibodies: FIG. 5A (NGF first and ADAMTS5 second), FIG. 5B (ADAMTS5 first and NGF second).

FIG. 6 provides a patent sequence table listing each of the constructs discussed herein together with the identification of amino acid and DNA sequences that comprise the heavy chains and light chains of the constructs.

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety, including U.S. Patent Application No. 63/404,273 and United States Patent Application Publication No. US2012-0095193 A1 and WO2012116260A1.

As used herein, the term “subject” should be construed to include subjects, for example medical or surgical subjects, such as humans and other animals requiring therapeutic intervention.

This description of the exemplary embodiments is intended to be read in connection with the accompanying figures, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the figure under discussion. These relative terms are for convenience of description and are not considered to be restrictive or limiting.

For purposes of the description hereinafter, it is to be understood that the embodiments described below may assume alternative variations and embodiments. It is also to be understood that the specific articles, compositions, and/or processes described herein are exemplary and should not be considered as limiting.

In the present disclosure the singular forms “a,” “an,” and “the” include the plural reference, and reference to a particular numerical value includes at least that particular value, unless the context clearly indicates otherwise. Thus, for example, a reference to “an antibody” or “an antibody fragment” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

As used herein, term “amino acid” broadly refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid.

As used herein, an “antibody fragment” includes a portion of an intact antibody, such as, for example, the antigen-binding or variable region of an antibody. Examples of antibody fragments include Fab, Fab′, F(ab′)2, and Fv fragments. For example, antibody fragments include isolated fragments, “Fv” fragments (consisting of the variable regions of the heavy and light chains), recombinant single chain polypeptide molecules in which light and heavy chain variable regions are connected by a peptide linker (“scFv proteins”), recombinant single domain antibodies consisting of a variable region of an antibody heavy chain (e.g., VHH), and minimal recognition units consisting of the amino acid residues that mimic a hypervariable region (e.g., a hypervariable region of a heavy chain variable region (VH), a hypervariable region of a light chain variable region (VL), one or more CDR domains within the VH, and/or one or more CDR domains within the VL). In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, heavy chain variable region, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively, or additionally, antigen binding fragment of an antibody may be wholly or partially synthetically produced. An antigen binding fragment of an antibody may optionally comprise a single chain antibody fragment.

As used herein, the term “binding” typically refers to a non-covalent association between or among two or more entities, unless expressed indicated otherwise, for example, referring to covalent bonding. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

The term “domain” is used herein to refer to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively, or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecular (e.g., a small molecule, carbohydrate, a lipid, a nucleic acid, or a polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

As used herein, the term “bispecific antibody” generally refers to an antibody having two distinct binding domains that can bind to two antigens or two epitopes (an antigen part) of one or more antigens simultaneously. Typically, a bispecific antibody containing at least two (or more) such segments is considered to be a bispecific antibody if the two segments are moieties that (1) are not included in nature in the same antibody, and/or (2) have not previously been linked to one another in a single antibody, and/or (3) have been linked to one another through action of the hand of man. Bispecific antibodies with defined specificities, as contemplated herein, are artificial molecules, per se not found in nature. They are generated by biochemical, molecular or genetic means. The generation of bispecific IgG molecules is difficult due to the fact that the antigen-binding sites are built by the variable domains of the light and heavy chain (VL, VH). A bispecific antibody requires two different heavy chains, and two different light chains, and exhibits asymmetry due to the presence of, at least, two different Fv regions. Unrestrained pairing of heavy and light chains of two antibodies expressed in one cell can theoretically result in 16 different combinations (10 different molecules), with only one being bispecific and the remaining pairings resulting in non-functional or monospecific molecules. To direct and to force correct assembly of correct binding sites, i.e., heavy and light chains, is one of the challenges of generating bispecific antibodies, and the inventors herein have overcome these challenges in the context of engineering novel bispecific antibodies for the treatment and management of chronic pain. (Brinkmann et al. The making of bispecific antibodies. MAbs. 2017 February/Mar;9 (2): 182-212. doi: 10.1080/19420862.2016.1268307. PMID: 28071970; PMCID: PMC5297537)

As used herein, “linker” refers to polypeptides typically added into antibody constructs most commonly to provide flexibility, fusion and/or increased distance between domains. For example, a glycine-serine (GS) repeat sequence is the most common linker sequence in the design of scFv-containing constructs due to their flexible nature. Inclusion of added linker sequence of various lengths between the VH/CHI and VL/CL domains of Dual Variable Domain (DVD) bispecific antibodies provide increased distance and flexibility between the two antigen binding domains and also act to fuse the binding domains together in a single amino acid chain. The linkers employed in DVDs are derived from native antibody sequence, which is believed to reduce the immunogenic potential of these constructs following administration.

As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues.

As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

The abbreviations or acronyms used in the present disclosure have corresponding meanings as those skilled in the art understand. For example, the term “scFv” refers to a single chain antibody fragment. The terms “anti-IFN-γ scFv” and “IFN-γ scFv” may be used interchangeably, and the term “anti-” refers to an entity or portion targeting or to be bound with another entity or portion. The term “anti-IFN-γ scFv” refer to a single chain antibody fragment targeting or to be bound with IFN-γ.

Chronic pain is one of the most prevalent and costly health problems in the world. According to recent studies, chronic pain (pain that persists past normal healing time, typically for more that 3 to 6 months) affects an estimated 20% of people worldwide, and accounts for 15-20% of physician visits. In addition to an individual's suffering, the economic consequences of dealing with chronic pain have widespread implications including for example, increased medical expenses, lost income, lost productivity, compensation payments, as well as legal ramifications.

A major category of chronic pain consists of arthritis, within which osteoarthritis (OA) is one of the most common forms. OA is a serious disease characterized by significant pain and impaired quality of life for over 500 million patients affected worldwide and drives an ever-growing burden on global health and economics. Current pharmacologic options to treat the condition focus on pain relief, but are limited by modest efficacy, safety concerns in the setting of chronic administration, and growing evidence that they may accelerate disease progression. Joint replacements are limited to large joints with end-stage structural disease and, although broadly used and effective for many patients, have many access restrictions to the procedure, can result in unfavorable long-term outcomes and are not an option for those with moderate, but highly symptomatic OA. Together, these observations highlight the urgent need to develop safer and more effective therapeutics to treat OA patients.

Pain and structural degeneration in OA are primarily driven by distinct and uncoupled pathways. This observation underscores the complex nature of the disease and helps explain the heterogeneity of patients impacted by the condition, as independent mechanisms may contribute to pain and disease onset/progression. The present disclosure provides a highly successful and novel therapeutic that both decreases pain and prevents (or reverses) structural degeneration. In certain embodiments, the invention disclosed herein is also able to remain in the joint thereby prolonging its efficacy and minimizing off-target effects. In certain embodiments, the bispecific antibodies described herein are able to alleviate pain and contribute to cartilage repair over an extended period of time. Though not wishing to be bound by the following theory, through simultaneously inhibiting two molecular targets in a single bispecific drug; nerve growth factor (NGF), a molecule which potently transmits pain, and a protease (which causes cartilage breakdown and serves as a tissue anchor to keep the drug at the site of disease), the inventors herein have developed a transformational medicine for the treatment of chronic pain and OA.

In certain embodiments, the molecular target of the bispecific drug comprises proteinases such as ADAMTS5, that cleave substrates located in the extracellular matrix of tissues comprising aggrecan, versican, biglycan, brevican, decorin, fibromodulin, lumican and the like which is associated with degenerative disease processes. Though not wishing to be bound by the following theory, it is possible to target the substrate (for example aggrecan) at the ADAMTS5 proteolysis site with one binding component of the antibody, which would have the same inhibitory and anchoring effect as targeting ADAMTS5 directly.

In certain embodiments, the compositions of the invention comprise the utilization of bispecific antibody platform technology expertise to generate and select novel, long-acting NGF/ADAMTS5, targeting therapeutic candidates for clinical development. The novel compositions described herein enable therapeutic intervention for addressing chronic pain, including OA, comprising the use of non-opioid therapeutics. In certain embodiments, the novel compositions herein may be used for treating OA pain following intra-articular administration. In certain embodiments, the compositions may be utilized to treat all OA joints via systemic administration. In additional embodiments, the compositions may qualify for a disease modification label for other chronic pain indications, including but not limited to lower back pain and cancer-related pain. The compositions are useful for both humans and non-human animals, including for example for use in the field of veterinary medicine to treat OA in dogs, cats, horses and other animals.

Provided herein are novel methods and compositions for treating chronic pain, wherein such novel methods and compositions simultaneously inhibit both pain and structural tissue degeneration. The compositions described herein are unique in that they comprise a single therapeutic molecule, and in certain embodiments, the compositions are capable of providing sustained relief. The compositions may be used to treat chronic pain, including, but not limited to arthritis, osteoarthritis, costochondritis, as well as other diseases and disorders associated with cartilage and bone issues. In certain embodiments, the compositions herein comprise a binding protein with two or more antigen binding components, wherein at least one antigen binding component is capable of binding to one more proteinases such as ADAMTS5 (which act on substrates, typically in the extracellular tissue matrix such as aggrecan, versican, biglycan, brevican, decorin, fibromodulin, lumican and the like), and wherein at least one antigen binding component is capable of binding to nerve growth factor (NGF). The antigen binding component may comprise an antibody, a monoclonal antibody, and/or (active) fragments thereof; the monoclonal antibody or fragment thereof may be mouse, chimeric, humanized, or fully human. In certain embodiments, the antigen binding component capable of binding to NGF may comprises a monoclonal antibody or Fc Fusion protein selected from the group including, but not limited to, tanezumab, fasinumab, fulranumab, TrkA Fc and/or p75NTR Fc.

In an embodiment, the novel compositions of the invention comprise a binding protein with two or more antigen binding components, wherein at least one antigen binding component is capable of binding to human ADAMTS5, and wherein at least one antigen binding component is capable of binding to nerve growth factor (NGF). The antigen binding component may comprise an antibody or fragment thereof; the antibody may comprise a monoclonal antibody or fragment thereof, wherein the monoclonal antibody or fragment thereof is mouse, chimeric, humanized, or fully human. In certain embodiments, the antigen binding components may comprise at least one complementarity determining region. The antigen binding components may selectively bind with high affinity to ADAMTS5. The antigen binding component capable of binding to NGF may comprise a monoclonal antibody selected from the group including, but not limited to, tanezumab, fasinumab, fulranumab, TrkA Fc and/or p75NTR Fc.

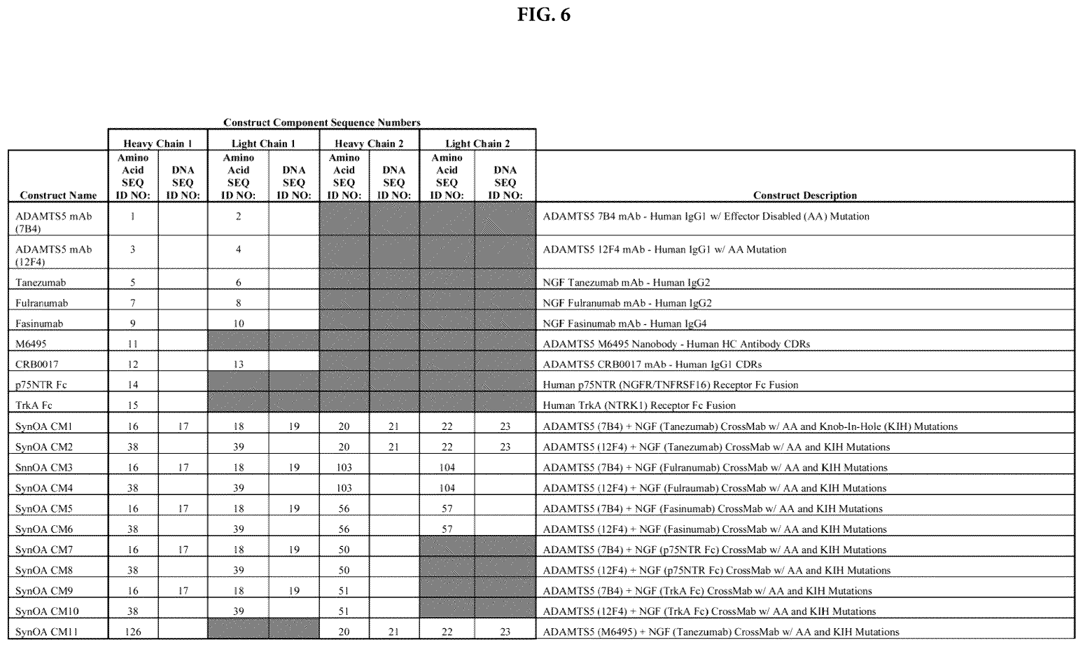

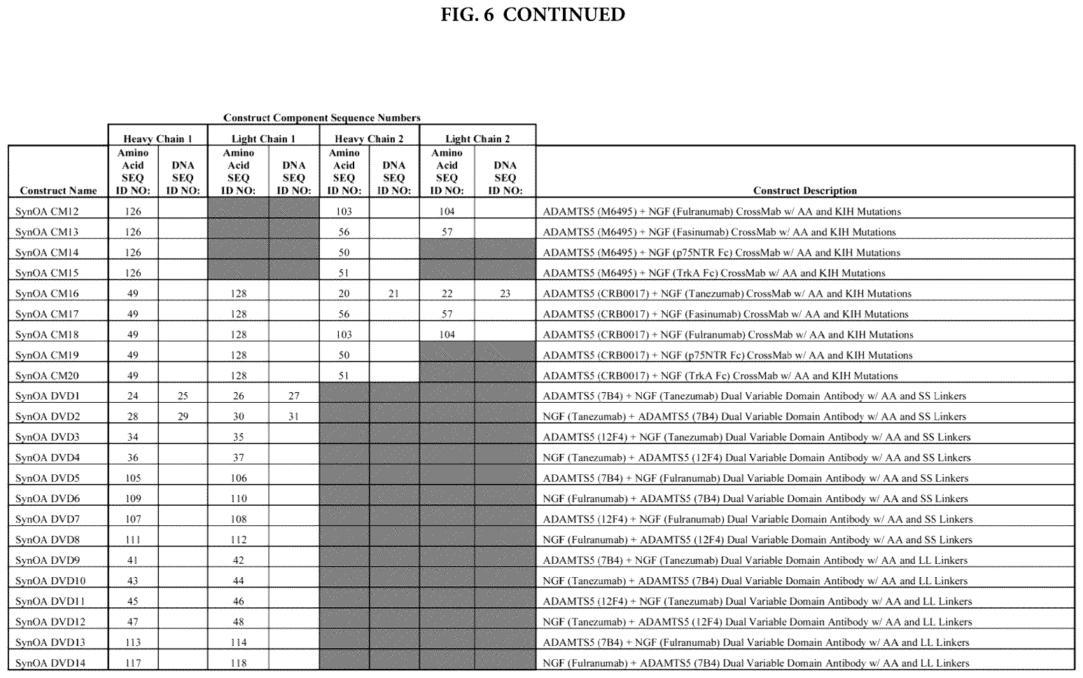

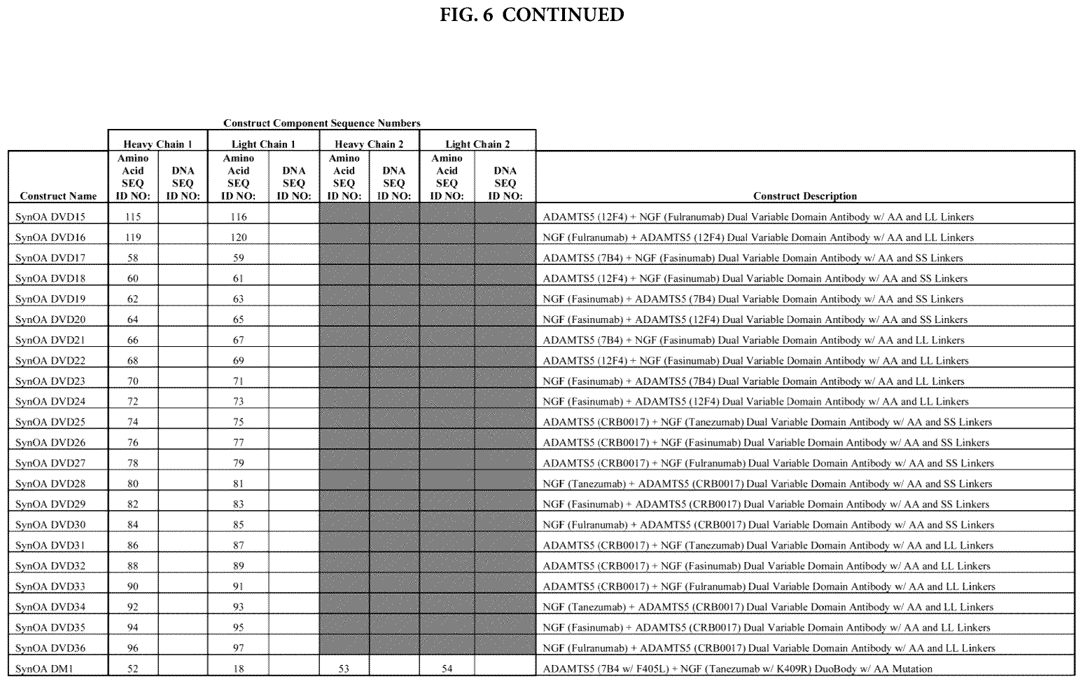

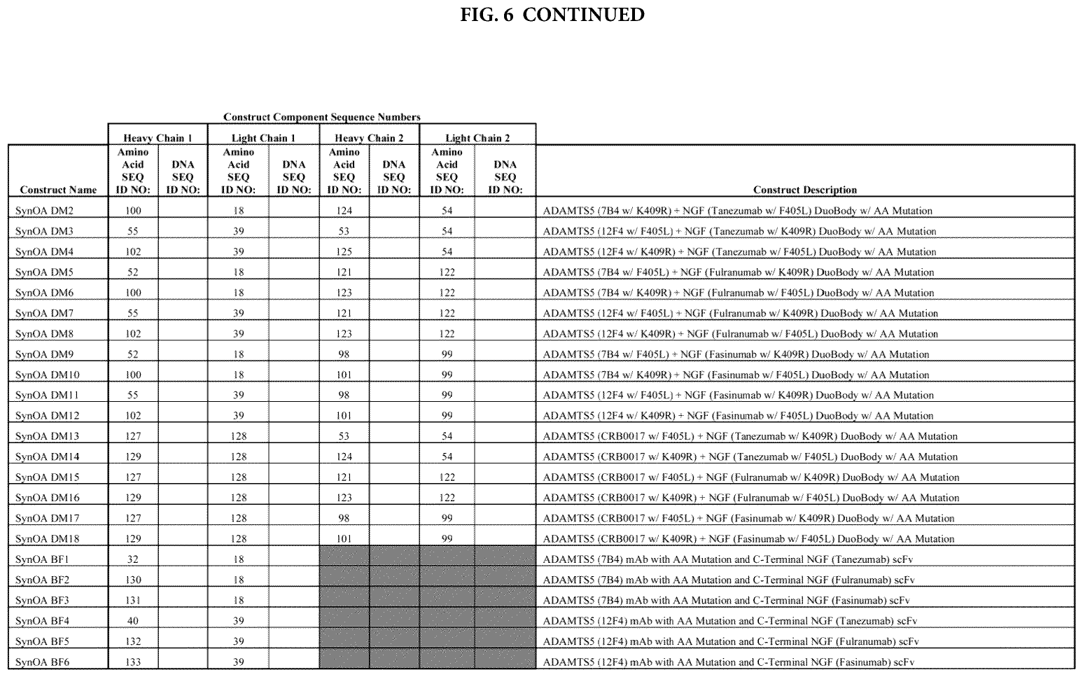

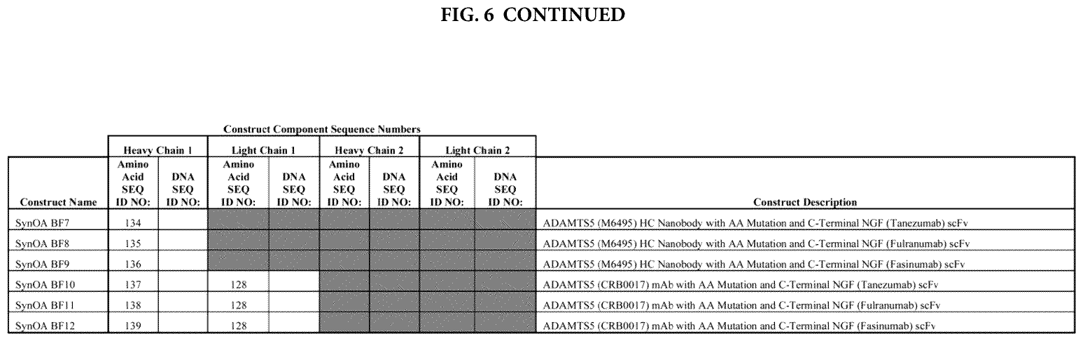

In certain embodiments, the novel composition comprises one antigen binding component that selectively binds with high affinity to one or more ADAMTS5 molecules and one antigen binding protein selectively binds with high affinity to one or more NGF molecules. Such a composition may comprise a bispecific antibody, represented by the constructs provided in FIG. 6.

In certain embodiments, the novel compositions and/or formulations of the invention may further comprise bone targeting agents, including but not limited to denosumab, and/or agents targeting RANK-L, romosozumab, and/or agents targeting sclerostin.

In certain embodiments, the novel compositions and/or formulations of the invention may further comprise vascular targeting agents, including but not limited, to bevacizumab, ramucirumab and/or agents targeting VEGF.

In certain embodiments, the novel compositions and/or formulations of the invention may further comprise TNF-alpha targeting agents, including but not limited, to adalimumab, infliximab and/or agents targeting TNF-alpha.

In certain embodiments, the novel compositions and/or formulations of the invention may further comprise interleukin targeting agents, including but not limited, to canakinumab, tocilizumab and/or agents targeting interleukins.

In certain embodiments, the novel compositions and/or formulations of the invention may further comprise an antigen binding protein capable of binding to an anti-tumor modulator, including, but not limited to PD-1.

In certain embodiments, the novel compositions of the invention may further comprise pharmaceutically acceptable excipients.

In an embodiment, provided herein are methods for treating the conditions of chronic pain, arthritis, or osteoarthritis in a subject, by administering to the subject a composition comprising a binding protein with two or more antigen binding components, wherein at least one antigen binding component is capable of binding to human ADAMTS5, and wherein at least one antigen binding component is capable of binding to nerve growth factor (NGF). In an embodiment, the composition may be administered intraarticularly, intravenously, intramuscularly, subcutaneously, orally, intranasally, and/or via respiratory inhaler. In an embodiment, the subject to be treated may be suffering from a disease of the cartilage.

In an embodiment, the novel compositions of the invention as disclosed herein, may be used to treat a subject suffering from one or more diseases selected from the group of: osteoarthritis, cancer, pain, chronic pain, neuropathic pain, postoperative pain, sports injuries, crosive arthritis, rheumatoid arthritis, psoriatic arthritis, Lyme arthritis, juvenile arthritis, ankylosing spondylosis, neuralgia, neuropathies, algesia, nerve injury, ischemia, neurodegeneration, inflammatory diseases, cartilage degeneration, diseases affecting the larynx, trachea, auditory canal, intervertebral discs, ligaments, tendons, joint capsules or bone development, intervertebral disc degeneration, osteopenia, or periodontal diseases, acute joint injury, and/or a disease related to joint destruction.

In certain embodiments, following administration of at least one dose of the novel compositions herein, the subject experiences a reduction in cartilage degradation.

In certain embodiments, following administration of at least one dose of the novel compositions herein, the subject experiences the inhibition or reduction in aggrecan proteolysis.

In an embodiment, provided herein are methods for treating osteoarthritis in a subject comprising the administration of a composition comprising: a protein with two or more antigen binding components, wherein at least one antigen binding component is capable of binding to human ADAMTS5, and wherein at least one antigen binding component is capable of binding to nerve growth factor (NGF). In an embodiment, the protein with two antigen binding components consists of a bispecific antibody, and one component thereof selectively binds with high affinity to ADAMTS5 and the other component thereof selectively binds with high affinity to NGF. The component that binds to ADAMTS5 is selected from the group including, but not limited to, constructs ADAMTS5 mAb (7B4), ADAMTS5 mAb (12F4), M6495, and CRB0017 (see FIG. 6) the component capable of binding to NGF comprises a monoclonal antibody selected from the group including, but not limited to, tanezumab, fasinumab, fulranumab, TrkA Fc and/or p75NTR Fc. The bispecific antibody is represented by constructs identified in FIG. 6. In certain embodiments, the composition may be administered intraarticularly, intravenously, intramuscularly, subcutaneously, orally, intranasally, and/or via respiratory inhaler. In certain embodiments, administration of the bispecific antibody composition enables the alleviation of pain and also the reduction of cartilage degradation. In certain embodiments, administration of the bispecific antibody composition results in extended relief. In certain embodiments, the subject is a vertebrate, including but not limited to human, non-human mammal, cat, dog, horse, cattle, rabbit, or cow.

In certain embodiments, the antigen binding component capable of binding to ADAMTS5 is characterized in that it inhibits ADAMTS5-mediated aggrecanase activity. In certain embodiments, the antigen binding component capable of binding to ADAMTS5 is characterized in that it inhibits the production of aggrecanase activity biomarkers, including but not limited to ARGS neoepitope. Administration of at least one dose of said composition may reduce cartilage degradation in said subject.

In certain embodiments, the administration of the novel bispecific antibody compositions of the invention improves the conditions of a subject suffering from at least one disease selected from the group consisting of: osteoarthritis, cancer, pain, chronic pain, neuropathic pain, postoperative pain, sports injuries, erosive arthritis, rheumatoid arthritis, psoriatic arthritis, Lyme arthritis, juvenile arthritis, ankylosing spondylosis, neuralgia, neuropathies, algesia, nerve injury, ischaemia, neurodegeneration, inflammatory diseases, cartilage degeneration, diseases affecting the larynx, trachea, auditory canal, intervertebral discs, ligaments, tendons, joint capsules or bone development, intervertebral disc degeneration, osteopenia, or periodontal diseases, acute joint injury, and/or a disease related to joint destruction.

In certain embodiments, the therapeutic bispecific antibody formulation of the invention comprises an injectable formulation, oral formulation, or intravenous formulation. The therapeutic bispecific antibody may be in a concentration ranging between 0.01 to 10 μg/mL, 0.5 to 10 μg/mL, 0.5 to 5 μg/mL, 0.5 to 4 μg/mL, 0.1 to 500 μg/mL, 1 to 400 μg/mL, 1 to 50 μg/mL, 10 to 20 μg/mL, 100 to 300 μg/mL, 125 and 175 μg/mL, 1 to 100 mg/mL, 20 to 80 mg/mL, 30 to 70 mg/mL, 40 to 70 mg/mL, 40 to 60 mg/mL. In certain embodiments, pharmaceutically acceptable excipients are present in a concentration range between 1 to 100 mg/mL, between 20 to 80 mg/mL, between 30 to 70 mg/mL, between 30 to 70 mg/mL and between 40 to 60 mg/mL.

In certain embodiments, methods for treating pain, arthritis or OA are provided wherein the novel compositions of the invention are administered to an organism at the site of pain, for example at a joint, such as in the neck, shoulder, elbow, wrist, fingers, hips, knees, spine or ankles. As such, methods for treating pain comprise administering a dose between 0.01 μg to 100 mg of a bispecific antibody composition to an organism having pain at the site of pain, the composition comprising: bispecific antibodies and pharmaceutically acceptable excipients. The compositions or formulations may be administered as single or multiple doses and the doses may be administered as needed, for example, they may be administered twice daily, daily, every other day, every third day, three times per week, twice per week, weekly, biweekly, monthly, bimonthly, quarterly, semi-annually, or annually. In certain embodiments, the compositions are engineered to have sustained release and prolonged effect The dose amount may be determined based on factors known to those skilled in the art (such as clinical factors, weight, pharmacokinetic profiles and conditions to be treated and may vary from about 1 μg to about 10 μg, about 10 μg to about 50 μg, about 50 μg to about 150 μg, about 150 μg to about 250 μg, about 250 μg to about 500 μg, about 500 μg to about 750 μg, about 750 μg to about 1 mg, about 1 mg to about 50 mg, about 1 mg to about 100 mg, about 50 mg to about 100 mg. In certain embodiments, the bispecific antibody may be present in a concentration of between 0.01 to 15 μg/mL, 0.01 to 10 μg/mL, 0.5 to 10 μg/mL, 0.5 to 5 μg/mL, or 0.5 to 4 μg/mL.

The compositions and formulations described herein may be administered by standard routes. In general, the compositions and formulations may be administered by the topical, intraarticular, transdermal, intraperitoneal, intracranial, intracerebroventricular, intracerebral, intravaginal, intrauterine, oral, rectal or parenteral (e.g., intravenous, intraspinal, subcutaneous or intramuscular, epidural) ophthalmic (including intravitreal or intracameral), nasal, topical (including buccal and sublingual), administration. route. In certain embodiments, osmotic minipumps may also be used to provide controlled delivery of therapeutic agents through cannulae to the site of interest. The biodegradable polymers and their use are described, for example, in detail in Brem et al., J. Neurosurg. 74:441-446 (1991), which is hereby incorporated by reference in its entirety.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Suitable dosages of the compositions and formulations disclosed herein will depend on the level of pain, condition or disease state being treated and other clinical factors such as weight and condition of the human or animal and the route of administration of the compound. It is to be understood that the invention has application for both human and veterinary use. The methods of the invention contemplate single as well as multiple administrations, given either simultaneously or over an extended period of time.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

The following examples are given to illustrate exemplary embodiments of the present disclosure. It should be understood, however, that the present disclosure is not to be limited to the specific conditions or details described in these examples. Examples are provided below to facilitate a more complete understanding of the invention. The following examples illustrate the exemplary modes of making and practicing the invention.

### EXAMPLES

### Example 1

### Bispecific Antibody Design

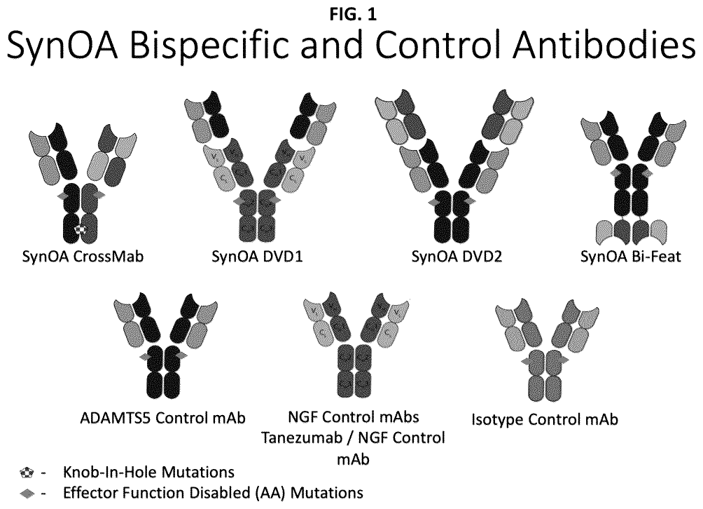

Sequences for the design of bispecific antibody constructs were derived from published monoclonal antibodies with single target specificity (SEQ ID NOS: 1-13) and/or human target receptor Fc fusion proteins (SEQ ID NOS: 14-15) and combined using multiple established bispecific antibody technology platforms. In some embodiments, the complementary determining regions (CDRs), and/or variable domains from the heavy and light chains of the published monoclonal antibodies were transferred from a human IgG2 framework into a human IgG1 framework. Where necessary and appropriate, mutations to the framework sequences were introduced to reduce immune effector function (AA mutant reducing ADCC, CDC activities) and/or enable efficient heterogeneous heavy and light chain pairing (for example, knob-in-hole, CH1-CL domain swapping and/or complementary heavy chain specific F405L and K409R). In some cases, linker sequences of various lengths were added between VH/CH1 and VL/CL domains (Dual Variable Domain bispecifics) and/or between the interface of CH3/VH and VH/VL domains (scFv containing constructs) to provide distance between target binding domains to increase binding potential or enable IgG/scFv fusion and VH/VL association within the scFv. Sequence ID numbers for the components of each bispecific antibody construct are summarized in Table 1 (SynOA Patent Sequence Table) and individual component sequences (SEQ ID NOS: 16-139) are appended (SynOA Bisepcific Ab Patent Sequences). Schematic representations of example bispecific and monoclonal control antibody constructs are shown in FIG. 1 below.

### Example 2

### Bispecific Antibody Construct Generation

Sequences of selected constructs were codon optimized at the DNA level for downstream mammalian cellular expression system compatibility using standard methods. Construct sequences were synthesized using as gBlock fragments (Integrated DNA Technologies, Inc) and cloned into mammalian expression vectors. The final constructs were then verified by forward and reverse DNA sequencing to confirm accuracy.

### Example 3

### Bispecific Antibody Expression and Purification

Expression vectors containing appropriate combinations to produce the desired bispecific antibodies were co-transfected into a mammalian expression cell line (CHO), and stable pools expressing and secreting the bispecific antibodies were established under selection with appropriate antibiotics. After expansion of the stable pools to appropriate volumes, the cell culture supernatants were collected for antibody purification using protein G agarose beads (Millipore, #16-266). Purified antibodies were dialyzed against PBS and the concentrations were measured using the BCA protein assay kit (ThermoFisher, #23250). Small fractions of the purified antibodies were assessed for purity and appropriate migration profiles on SDS-PAGE gels under reducing and non-reducing conditions, and the remaining antibodies were aliquoted and stored at −80° C.

### Example 4

### Bispecific Antibody Target Binding and Affinity Measurements

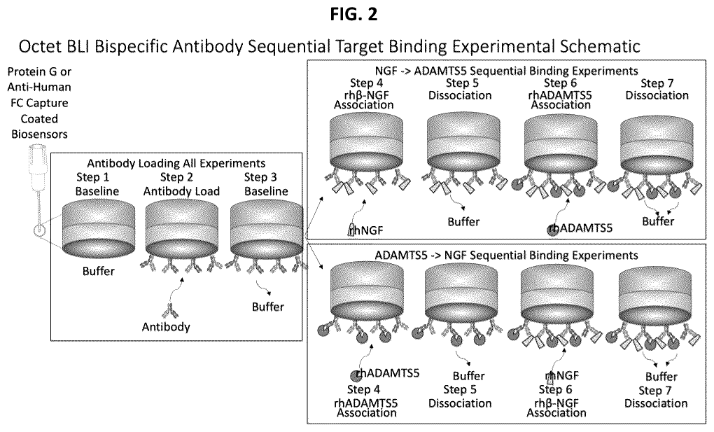

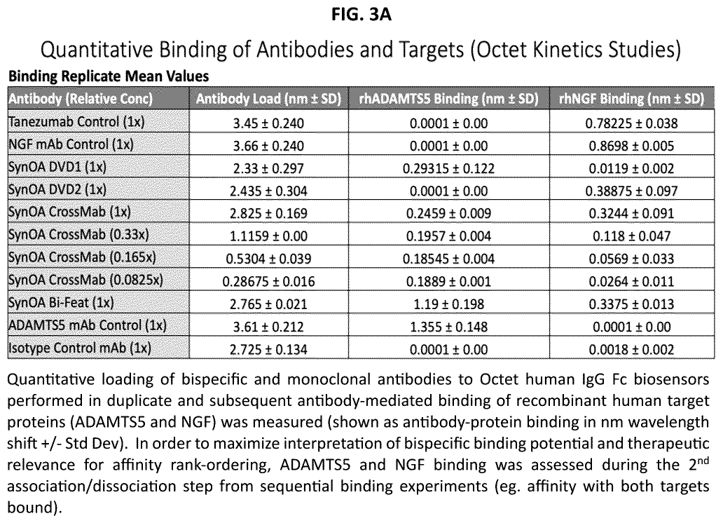

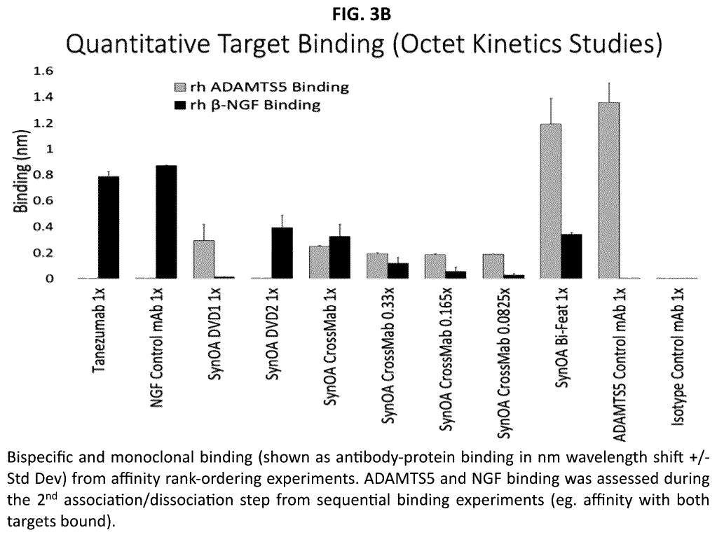

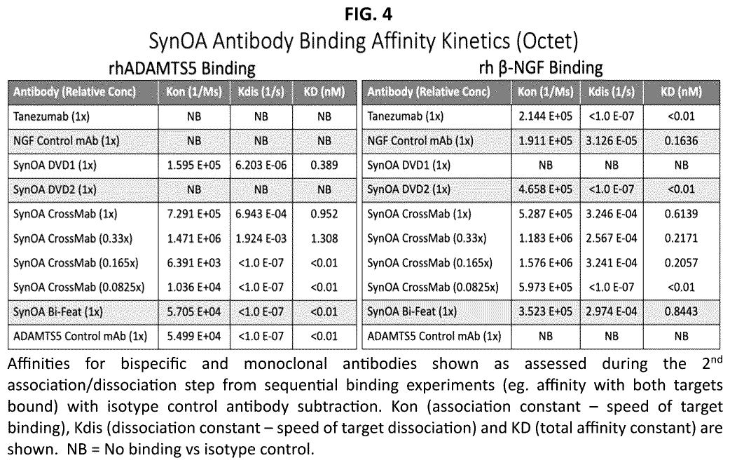

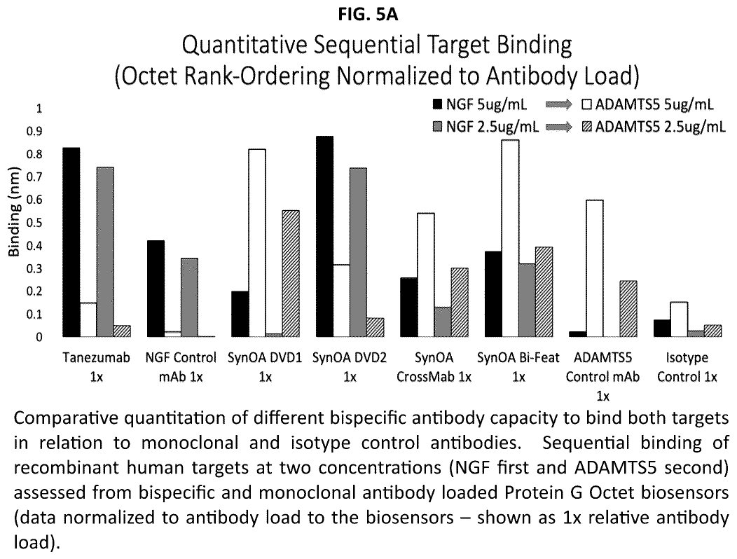

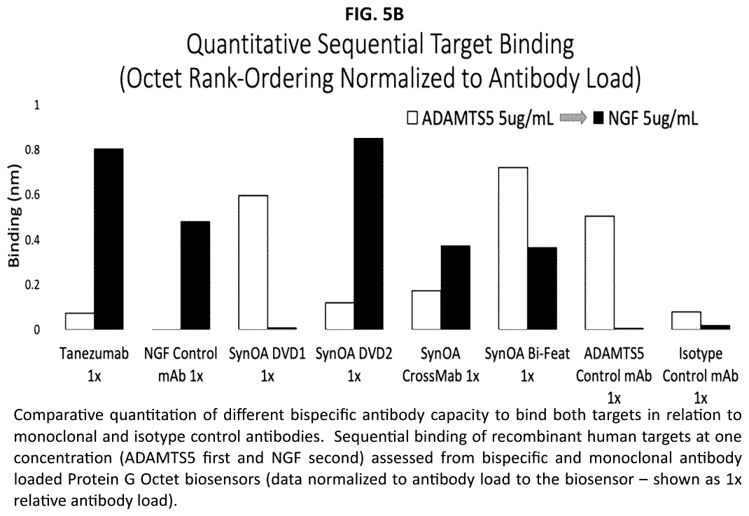

Binding and affinity kinetics of bispecific and monoclonal control antibodies to the target human proteins were measured using a Bio-Layer Interferometry (BLI) technology platform (Octet RH16, Sartorius) to enable characterization and rank ordering. For all experiments, Protein G and/or Anti-Human Fc-Capture coated Octet biosensors were first calibrated for loading of each bispecific and control antibody to identify comparable loading capacity. Subsequent target-specific real-time binding and affinity experiments using comparable antibody loading concentrations from calibrations were performed in multiple experiments for each target in sequential order using recombinant human ADAMTS5 (R&D Systems Cat #2198-AD) and recombinant human beta-NGF (R&D Systems Cat #256-GF-100/CF) each separated by a 10-minute PBS/Kinetics buffer wash/dissociation step (shown schematically in FIG. 2). To measure the ability of the bispecific antibodies to bind to both targets simultaneously and assess the potential of each target once bound to influence the binding stability of the other target, the sequential order of target addition was alternated in independent experiments. Monoclonal antibodies specific for each target (ADAMTS5 or beta-NGF), isotype-matched antibodies with specificities to targets not included in the experiments and buffer-only loading conditions were run as positive and negative controls, respectively. Experiments were designed and run using Octet Data Acquisition Software and all experiments were performed at 30° C. with continuous shaking of the reagent plate at 1000 RPM. In some experiments a dose range of antibody loading and/or target protein concentration was used to enable interpretation of results. Once acquired, the experimental data was analysed for quantitative antibody loading and target binding (measured as nm shift) and affinity kinetics (association [Kon], dissociation [Kdis] and equilibrium dissociation [KD] constants) using Octet Data Analysis Software (FIG. 2). Values reported for binding and affinity rank order characterization were calculated following the second sequential binding/association step for each target (e.g. with both targets bound) to maximize stringency and interpretation of therapeutic relevance (FIG. 3A and FIG. 3B). For affinity kinetics studies, the isotype control antibody loaded biosensors were used as a reference in the calculations to control for non-specific target binding (FIG. 4). Additional studies were conducted in order to assess comparative quantitation of different bispecific antibody capacity to bind both targets in relation to monoclonal and isotype control antibodies (FIG. 5A and FIG. 5B).

### Example 5

### Bispecific Antibody Functional Inhibition of ADAMTS5 Activity

Inhibition of ADAMTS5-mediated proteolysis of its native substrates (including, but not limited to, Aggrecan, Brevican, Versican) by antibodies, and other inhibitors, can be measured using multiple experimental assays to calculate and rank order the potency of inhibitors by IC50s (half maximal inhibitory concentrations). Functional ADAMTS5 assays typically involve incubating a fixed concentration of purified recombinant ADAMTS5 (human or other species) with a fixed concentration of a purified, recombinant or peptide version of a native substrate (most commonly aggrecan) in an assay buffer (containing ZnCl) at a physiological pH and temperature to allow for proteolysis of the substrate to progress. ADAMTS5 inhibitors are tested by conducting these assays in separate assay wells or tubes in the presence or absence of each inhibitor in a concentration dose-range to derive a functional potency IC50 calculation. Following a given time, the reaction is stopped by addition of an EDTA-containing solution and proteolysis of the substrate is detected.

ADAMTS5 mediated proteolysis in these experiments can be detected in numerous ways depending on the substrate used:
1) Purified and recombinant substrates-visual mobility of cleaved and uncleaved substrate in each assay sample following polyacrylamide gel electrophoresis and staining of the gel with a total protein dye.
2) Peptide substrate-Typically fluorescence resonance energy transfer (FRET) based assays that employ dual donor and acceptor fluorescence-tagged peptides containing the proteolytic cleavage site which emit a fluorescent signal when the peptide is cleaved. One example of an ADAMTS5 substrate peptide is WAAG-3R (Anaspec, Cat #60431-1).
3) All substrate forms-Using a neoepitope monoclonal antibody (such as OA-1 or BC-3 for the aggrecan ARGS neoepitope) that recognize an epitope that is generated following ADAMTS5-mediated proteolysis. This can be performed by western blotting or in various immunoassay-based methods for quantitation.

Each inhibitor and test condition is visualized by plotting the measured value or percent inhibition of each assay condition on the y-axis and the inhibitor concentration (log scale) on the x-axis and the IC50 can be calculated manually or by using software (such as GraphPad Prism, RStudio or MS Excel).

### Example 6

### Bispecific Antibody Functional Inhibition of beta-NGF Activity

Inhibition of beta-NGF activity to derive an IC50 potency for rank ordering can be measured using an NGF receptor (TrkA [NTRK1] and/or p75NTR [NGFR/TNFRSF16]) expressing reporter cell line (such as the commercial TRKA Human RTK Kinase Cell-Based Antagonist Functional Assay from DiscoverX). To perform these assays a fixed concentration of recombinant human beta-NGF is pre-incubated for up to 1 hour at a physiological temperature in the presence or absence of a concentration dose range of the inhibitor and the NGF receptor reporter cell line is then treated with the sample in an assay plate. Following a subsequent incubation period, the reporter signal (typically by luminescence) is detected and the resulting data can be visualized by plotting the measured value or percent inhibition of each assay condition on the y-axis and the inhibitor concentration (log scale) on the x-axis and the IC50 for each inhibitor is calculated manually or by using software (such as GraphPad Prism, RStudio or MS Excel).

Source mAb Sequences for SynOA
bispecific antibodies
SEQ ID NO: 1:
Amino Acid 7B4 Heavy Chain:
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSRDELTKNQVSLTCLVKGFYPSDI
AVEWESNGQPENNYKTTPPVLDSDGSFFLYSKLTVDKSRWQQGNVFSC
SVMHEALHNHYTQKSLSLSPGK
SEQ ID NO: 2:
Amino Acid 7B4 Light Chain:
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 3:
Amino Acid 12F4 Heavy Chain:
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNH
YTQKSLSLSPGK
SEQ ID NO: 4:
Amino Acid 12F4 Light Chain:
DIQMTQSPSSLSASVGDRVTITCKASQSVGTTIVWTQQLPGKAPKLLI
YSASNRHTGVPSRFSGSGSGTDFTLTISSLQPEDFATYYCQQYTSYPF
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 5:
Amino Acid Tanezumab Heavy Chain:
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPSVFPLAPCSRSTSESTA
ALGCLVKDYFPEPVTVSWNSGALTSGVHTFPAVLQSSGLYSLSSVVTV
PSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAK
TKPREEQFNSTFRVVSVLTVVHQDWLNGKEYKCKVSNKGLPSSIEKTI
SKTKGQPEPQVYTLPPSREEMTKNQVSLTCLVKGFYPSDIAVEWESNG
QPENNYKTTPPMLDSDGSFFLYSKLTVDKSRWQQGNVFSCSVMHEALH
NHYTQKSLSLSPGK
SEQ ID NO: 6:
Amino Acid Tanezumab Light Chain:
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPSVFIFPP
SDEQLKSGTASVVCLLNNFYPREAKVQWKVDNALQSGNSQESVTEQDS
KDSTYSLSSTLTLSKADYEKHXVYACEVTHQGLSSPVTKSFNRGEC
SEQ ID NO: 7:
Amino Acid Fulranumab Heavy chain
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPSVFPLAPCSRSTSES
TAALGCLVKDYFPEPVTVSWNSGALTSGVHTFPAVLQSSGLYSLSSVV
TVPSSNFGTQTYTCNVDHKPSNTKVDKTVERKCCVECPPCPAPPVAGP
SVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWE
SNGQPENNYKTTPPMLDSDGSFFLYSKLTVDKSRWQQGNVFSCSVMHE
ALHNHYTQKSLSLSPGK
SEQ ID NO: 8:
Amino Acid Fulranumab Light chain
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 9:
Amino Acid Fasinumab Heavy chain
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPSVFPLAPCSRSTSESTAALG
CLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHN
HYTQKSLSLSLGK
SEQ ID NO: 10:
Amino Acid Fasinumab Light chain
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 11:
Amino Acid M6495 Heavy Chain CDRs
CDRH1 GRTVSSYAMG;
CDRH2 GISRSAERTY;
CDRH3 DLDPNRIFSREEYAY
SEQ ID NO: 12:
Amino Acide CRB0017 Heavy Chain CDRs
CDRH1-SYWMH;
CDRH2-YINPSTGYTEYNQKFK;
CDRH3-GGYDDLGY
SEQ ID NO: 13:
Amino Acid CRB0017 Light Chain CDRs
CDRL1-RSSKSLLYKDGKTYLY;
CDRL2-LMSTRAS;
CDRL3-QQLVUYPYT
SEQ ID NO: 14:
P75NTR IgG1 Fc Sequence w AA mutation
KEACPTGLYTHSGECCKACNLGEGVAQPCGANQTVCEPCLDSVTFSDV
VSATEPCKPCTECVGLQSMSAPCVEADDAVCRCAYGYYQDETTGRCEA
CRVCEAGSGLVFSCQDKQNTVCEECPDGTYSDEANHVDPCLPCTVCED
TERQLRECTRWADAECEEIPGRWITRSTPPEGSDSTAPSTQEPEAPPE
QDLIASTVAGVVTTVMGSSQPVVTRGTTDNDIEGRMDPKSCDKTHTCP
PCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQ
GNVFSCSVMHEALHNHYTQKSLSLSPGK
SEQ ID NO: 15:
TrkA IgG1 Fc Sequence w AA mutation
AAPCPDACCPHGSSGLRCTRDGALDSLHHLPGAENLTELYIENQQHLQ
HLELRDLRGLGELRNLTIVKSGLRFVAPDAFHFTPRLSRLNLSFNALE
SLSWKTVQGLSLQELVLSGNPLHCSCALRWLQRWEEEGLGGVPEQKLQ
CHGQGPLAHMPNASCGVPTLKVQVPNASVDVGDDVLLRCQVEGRGLEQ
AGWILTELEQSATVMKSGGLPSLGLTLANVTSDLNRKNVTCWAENDVG
RAEVSVQVNVSFPASVQLHTAVEMHHWCIPFSVDGQPAPSLRWLFNGS
VLNETSFIFTEFLEPAANETVRHGCLRLNQPTHVNNGNYTLLAANPFG
QASASIMAAFMDNPFEFNPEDPIPVSFSPVDTNSTSGDPVEIEGRIDP
KSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKN
QVSLTCLVKGFYPSDIAVEWESNGQPENNYKTTPPVLDSDGSFFLYSK
LTVDKSRWQQGNVFSCSVMHEALHNHYTQKSLSLSPGK
SynOA Prototype Sequences
(Incl. AA and DNA Sequences)
SEQ ID NO: 16
SynOA CM1-7B4 Amino Acid Heavy Chain
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVCTLPPSRDELTKNQVSLSCAVKGFYPSDI
AVEWESNGQPENNYKTTPPVLDSDGSFFLVSKLTVDKSRWQQGNVFSC
SVMHEALHNHYTQKSLSLSPGK
SEQ ID NO: 17
SynOA CM1-7B4 DNA Heavy Chain
GAAGTCCAACTTGTGGAAAGTGGTGGGGGACTTGTGCAACCTGGCGGA
TCACTCCGTCTCAGTTGTGCCGCTAGTGGTTTTACCTTTTCCGATGCT
TGGATGGATTGGGTTAGACAAGCTCCTGGGAAAGGATTGGAGTGGGTT
GCAGAAATCCGCAACAAGGCAAATAACCACGCCAGACACTACGCTGAG
AGTGTCAAAGGTCGCTTTACTATAAGTAGAGATAACGCCAAAAATAGT
CTTTACTTGCAAATGAACTCCCTCCGGGCCGAGGACACTGCTGTTTAC
TACTGCGCAAGGACATACTATTACGGTAGCTCATACGGATATTGTGAT
GTTTGGGGACAAGGTACACTCGTTACCGTCAGTAGCGCTAGCACTAAG
GGGCCATCAGTATTCCCCCTGGCTCCCAGCTCCAAATCTACCTCAGGA
GGAACAGCAGCTCTGGGTTGCCTTGTAAAAGACTACTTTCCTGAACCT
GTTACTGTTAGCTGGAATAGTGGAGCTCTTACAAGCGGCGTACATACT
TTCCCTGCCGTGTTGCAGTCTAGTGGTCTTTACTCTCTTTCTTCTGTA
GTAACTGTACCTAGTAGCTCCTTGGGTACACAAACCTACATATGTAAC
GTCAATCATAAGCCTAGCAATACCAAAGTAGATAAAAAAGTTGAGCCT
AAAAGTTGTGACAAAACCCACACTTGCCCTCCCTGCCCTGCACCCGAG
GCCGCAGGGGGACCCTCAGTCTTCCTGTTTCCTCCAAAACCTAAAGAC
ACTTTGATGATATCCAGGACCCCCGAGGTAACCTGTGTGGTTGTAGAT
GTTAGCCATGAAGATCCCGAGGTAAAGTTCAACTGGTATGTTGATGGG
GTAGAGGTGCATAACGCTAAAACTAAACCTCGGGAGGAACAGTACAAT
TCAACTTACAGGGTTGTAAGCGTACTCACAGTCCTGCACCAAGACTGG
CTGAATGGCAAGGAGTATAAATGTAAAGTATCTAACAAGGCACTGCCC
GCTCCTATTGAAAAGACAATATCAAAAGCTAAGGGGCAGCCACGAGAA
CCCCAGGTGTGCACCCTCCCCCCATCCAGAGATGAACTTACCAAAAAT
CAAGTGAGCTTGTCCTGCGCTGTCAAAGGATTCTATCCTTCAGATATA
GCCGTGGAGTGGGAAAGTAACGGCCAGCCTGAAAACAATTATAAGACA
ACACCACCCGTGCTGGACAGCGATGGTAGTTTTTTTCTGGTCAGCAAG
TTGACTGTAGATAAGTCTCGATGGCAGCAGGGCAATGTTTTCAGTTGT
TCTGTAATGCACGAAGCTCTTCATAATCACTATACTCAGAAGTCTCTT
TCACTTAGCCCCGGCAAA
SEQ ID NO: 18
SynOA CM1-7B4 Amino Acid Light Chain
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 19
SynOA CM1-7B4 DNA Light Chain
GACATACAAATGACTCAATCTCCATCTTCCTTGTCTGCTTCAGTAGGA
GACCGAGTTACAATTACTTGCCGCACTTCCGAGAACATTTACTCATAT
CTGGCTTGGTATCAGCAAAAGCCAGGTAAGGCCCCAAAACTCCTTATC
TATAATGCTAAGACTCTTGCTGAGGGAGTACCCAGTAGGTTTTCCGGT
TCCGGTTCAGGCACAGATTTCACACTCACTATTTCTTCACTCCAGCCT
GAGGACTTCGCTACTTATTATTGCCAACATCACTACGGGACACCCTGG
ACTTTCGGGCAAGGGACTAAACTGGAAATAAAACGTACCGTGGCTGCC
CCAAGTGTCTTCATATTTCCCCCATCTGATGAGCAACTCAAATCAGGT
ACTGCTTCTGTTGTTTGCCTTCTCAACAATTTTTATCCACGTGAGGCA
AAAGTCCAGTGGAAGGTGGACAACGCCCTGCAATCAGGTAACAGTCAG
GAGTCAGTGACAGAACAAGATAGCAAAGACAGTACTTATTCCCTTTCC
AGCACCCTGACCCTCTCTAAAGCTGACTATGAGAAACATAAGGTCTAC
GCCTGTGAAGTAACACATCAAGGTCTTTCATCTCCAGTCACCAAGTCT
TTCAACAGGGGGGAGTGT
SEQ ID NO: 20:
NGF mAb1 (Tanezumab) Amino Acid Heavy Chain:
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASVAAPSVFIFPPSDEQLKSGTA
SVVCLLNNFYPREAKVQWKVDNALQSGNSQESVTEQDSKDSTYSLSST
LTLSKADYEKHKVYACEVTHQGLSSPVTKSFNRGECDKTHTCPPCPAP
EAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKAL
PAPIEKTISKAKGQPREPQVYTLPPCRDELTKNQVSLWCLVKGFYPSD
IAVEWESNGQPENNYKTTPPVLDSDGSFFLYSKLTVDKSRWQQGNVFS
CSVMHEALHNHYTQKSLSLSPGK
SEQ ID NO: 21:
Tanezumab DNA Heavy Chain
CAGGTGCAGCTCCAAGAATCAGGCCCAGGCTTGGTGAAACCCTCAGAA
ACTCTCAGCCTCACATGCACTGTGTCTGGCTTTAGCCTGATTGGTTAT
GACCTCAACTGGATTCGTCAACCCCCCGGCAAGGGTCTGGAATGGATC
GGAATCATCTGGGGAGATGGAACTACAGACTACAATAGTGCCGTAAAG
TCCCGAGTGACAATAAGTAAAGACACATCCAAGAACCAGTTTTCACTC
AAACTTTCCTCAGTCACCGCTGCTGACACAGCAGTCTATTATTGCGCA
AGAGGTGGATACTGGTATGCTACAAGCTACTACTTCGATTACTGGGGA
CAGGGTACCCTTGTGACCGTATCCTCCGCATCCGTGGCAGCCCCTAGT
GTATTCATCTTCCCTCCTTCAGATGAGCAACTCAAGTCAGGAACTGCC
TCAGTCGTGTGTTTGTTGAATAACTTCTACCCACGTGAAGCCAAAGTC
CAATGGAAGGTCGATAATGCCTTGCAATCCGGGAACAGCCAAGAGTCA
GTGACCGAGCAGGACAGTAAAGACAGTACTTACTCTTTGTCATCTACC
CTTACCCTTTCCAAGGCTGACTACGAGAAACATAAGGTGTACGCTTGC
GAAGTAACTCACCAGGGACTCAGTAGCCCAGTTACCAAATCATTCAAC
AGAGGAGAATGTGATAAGACTCATACCTGCCCCCCTTGTCCTGCTCCC
GAGGCTGCTGGGGGCCCATCTGTCTTTCTGTTTCCCCCAAAGCCTAAG
GATACTCTGATGATTTCTCGAACTCCCGAGGTCACTTGCGTAGTAGTA
GACGTCAGTCACGAAGACCCAGAAGTCAAGTTCAATTGGTATGTAGAT
GGGGTAGAAGTGCATAATGCTAAGACTAAACCTCGAGAGGAGCAGTAC
AACTCAACTTACAGGGTCGTTAGCGTATTGACCGTCCTCCATCAAGAT
TGGCTCAACGGAAAAGAATACAAATGTAAAGTGTCTAATAAGGCCCTG
CCCGCACCTATCGAAAAAACAATCTCTAAGGCTAAAGGGCAGCCACGT
GAACCACAAGTATATACTCTGCCACCTTGCCGCGATGAGTTGACTAAA
AACCAGGTGAGTTTGTGGTGTTTGGTAAAAGGCTTTTACCCTTCTGAC
ATAGCCGTGGAATGGGAGTCCAACGGGCAACCAGAGAACAATTACAAA
ACAACTCCTCCTGTCCTGGACAGTGACGGTTCTTTTTTTCTCTATTCA
AAGCTGACCGTGGACAAGTCAAGATGGCAACAGGGCAACGTCTTCTCT
TGTAGTGTGATGCACGAAGCCTTGCATAATCATTATACCCAGAAATCC
CTTTCACTCTCTCCTGGTAAG
SEQ ID NO: 22
SynOA CM1-Tanezumab Amino Acid Light Chain:
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKVTVLSSASTKGPSVFPLAPSSKSTSGGTAALGCLVKDYFPE
PVTVSWNSGALTSGVHTFPAVLQSSGLYSLSSVVTVPSSSLGTQTYIC
NVNHKPSNTKVDKKVEPKSC
SEQ ID NO: 23:
SynOA CM1-Tanezumab DNA Light Chain
GACATACAGATGACACAGAGCCCCTCTTCCTTGTCTGCTTCTGTGGGT
GATCGCGTCACAATTACCTGCCGCGCTAGTCAATCCATATCAAACAAC
CTGAACTGGTATCAACAGAAGCCCGGAAAGGCCCCCAAGCTCCTCATT
TACTACACATCCAGATTCCACTCCGGTGTTCCATCTAGGTTCAGCGGC
TCAGGGTCTGGCACAGACTTCACATTCACAATCAGCTCACTTCAGCCC
GAGGACATAGCTACATACTATTGTCAGCAGGAACACACACTCCCTTAC
ACATTTGGCCAAGGAACCAAAGTAACTGTACTTTCATCCGCAAGCACT
AAGGGTCCATCAGTCTTTCCACTTGCACCTTCATCTAAATCCACCTCA
GGTGGTACTGCAGCACTCGGTTGTTTGGTAAAAGATTACTTTCCTGAG
CCAGTGACTGTTAGCTGGAATAGTGGAGCCCTTACCTCCGGTGTCCAT
ACATTCCCCGCTGTGTTGCAATCATCTGGTCTCTATTCCCTTTCATCT
GTAGTTACCGTCCCTAGCTCCTCTCTGGGCACACAAACATATATTTGT
AACGTGAACCATAAACCCTCCAACACCAAAGTTGATAAGAAGGTCGAG
CCCAAATCTTGT
SEQ ID NO: 24:
SynOA DVD1 Amino Acid Heavy Chain
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPQVQLQESGPGLVKP
SETLSLTCTVSGFSLIGYDLNWIRQPPGKGLEWIGIIWGDGTTDYNSA
VKSRVTISKDTSKNQFSLKLSSVTAADTAVYYCARGGYWYATSYYFDY
WGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNV
NHKPSNTKVDKKVEPKSCDKTHTCPPCPAPEAAGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGK
SEQ ID NO: 25:
SynOA DVD1 DNA Heavy Chain
GAAGTACAATTGGTGGAAAGCGGCGGAGGCCTTGTCCAACCTGGCGGT
TCTTTGCGGCTGTCCTGTGCCGCTAGTGGTTTTACCTTTAGTGATGCC
TGGATGGACTGGGTGCGGCAGGCCCCTGGTAAGGGCCTGGAATGGGTC
GCTGAAATAAGAAATAAGGCAAATAACCATGCAAGACATTACGCTGAA
AGTGTGAAGGGCAGGTTCACTATATCCCGAGATAACGCAAAGAACAGT
TTGTACCTTCAGATGAACTCCTTGCGTGCCGAGGATACCGCCGTCTAC
TACTGCGCTCGCACATACTACTACGGTTCATCATACGGGTACTGCGAC
GTCTGGGGGCAAGGAACACTTGTCACCGTTTCTTCTGCCTCTACTAAG
GGACCCCAGGTTCAGTTGCAGGAGTCCGGGCCCGGTCTGGTAAAGCCT
TCTGAAACCCTGAGTTTGACCTGTACCGTGTCCGGTTTCTCACTGATT
GGCTATGATCTCAACTGGATACGACAGCCCCCCGGGAAGGGCCTGGAG
TGGATTGGCATCATCTGGGGAGATGGAACCACCGACTACAACTCCGCT
GTCAAAAGTAGAGTGACTATTTCCAAAGACACCAGCAAGAACCAATTC
TCACTGAAATTGAGTTCAGTTACAGCTGCCGACACTGCTGTTTACTAT
TGTGCAAGAGGGGGATATTGGTATGCCACTTCCTATTATTTTGACTAT
TGGGGCCAGGGAACACTTGTAACAGTATCCTCAGCCTCAACAAAAGGA
CCATCTGTATTTCCCCTTGCACCCAGTTCAAAGTCTACTTCAGGTGGG
ACTGCCGCTCTCGGCTGTCTGGTAAAAGATTATTTCCCCGAGCCAGTT
ACTGTAAGTTGGAATAGCGGTGCACTGACAAGTGGGGTCCATACTTTC
CCTGCCGTGCTTCAGAGTTCCGGCCTTTATTCACTCTCTAGTGTAGTT
ACCGTTCCCTCATCTAGCCTTGGAACCCAAACCTATATCTGTAATGTG
AATCATAAACCATCTAATACTAAGGTTGATAAAAAGGTTGAACCTAAG
AGCTGCGATAAAACACATACCTGTCCTCCTTGTCCTGCTCCTGAAGCT
GCTGGTGGACCTTCAGTGTTTCTGTTCCCCCCCAAGCCAAAAGATACA
CTGATGATCTCTCGTACTCCTGAGGTTACTTGTGTCGTCGTCGATGTG
TCACACGAAGATCCCGAAGTGAAGTTCAACTGGTACGTCGATGGTGTT
GAAGTACACAATGCCAAAACAAAACCCAGAGAGGAGCAATACAATAGT
ACATACCGCGTAGTGTCTGTTCTCACCGTATTGCATCAGGACTGGCTG
AACGGTAAAGAGTACAAATGTAAAGTTTCTAATAAAGCACTCCCAGCC
CCTATAGAAAAGACCATCAGCAAAGCTAAAGGGCAGCCCCGTGAGCCA
CAAGTCTACACATTGCCTCCTAGTAGGGATGAGCTGACAAAAAATCAA
GTGTCCCTGACCTGCCTCGTGAAAGGATTTTATCCTTCTGATATAGCA
GTTGAATGGGAATCCAACGGTCAACCTGAGAATAATTATAAGACCACT
CCCCCAGTGCTGGATTCTGATGGCAGTTTCTTCTTGTATTCTAAATTG
ACTGTCGACAAATCCCGTTGGCAACAGGGAAATGTGTTTTCATGCTCT
GTTATGCACGAGGCTTTGCACAATCACTACACTCAGAAGTCCCTGAGC
TTGTCTCCAGGAAAA
SEQ ID NO: 26:
SynOA DVD1 Amino Acid Light Chain
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPDIQMTQSPSSLSASVGDRVTITCRASQSISN
NLNWYQQKPGKAPKLLIYYTSRFHSGVPSRFSGSGSGTDFTFTISSLQ
PEDIATYYCQQEHTLPYTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 27:
SynOA DVD1 DNA Light Chain
GACATACAGATGACACAGTCTCCTAGCTCCCTGTCCGCAAGCGTTGGA
GATAGAGTCACAATAACTTGTAGAACATCAGAAAACATATATAGCTAC
TTGGCTTGGTACCAACAGAAGCCTGGTAAGGCACCCAAGCTCCTCATA
TACAACGCCAAGACCCTTGCCGAGGGTGTGCCAAGCAGATTCAGCGGG
TCAGGGAGCGGGACCGATTTTACACTTACTATTTCCTCTTTGCAGCCC
GAAGACTTTGCTACTTACTACTGTCAGCACCACTACGGCACTCCTTGG
ACTTTCGGACAAGGTACTAAACTTGAAATAAAGAGGACCGTAGCCGCC
CCCGATATACAGATGACTCAAAGTCCTAGTTCCCTTTCCGCTAGCGTA
GGAGACCGCGTAACAATTACCTGTAGAGCATCACAAAGTATAAGCAAC
AATCTGAACTGGTACCAGCAGAAACCTGGGAAGGCCCCTAAGTTGCTG
ATCTATTACACCTCCAGGTTTCATAGTGGCGTACCATCTCGTTTCAGT
GGATCAGGTTCTGGCACCGATTTCACTTTTACCATATCCTCACTCCAG
CCCGAAGATATCGCTACCTACTATTGTCAACAGGAACACACCCTTCCT
TATACATTTGGGCAGGGGACCAAGCTTGAGATCAAACGGACTGTGGCA
GCACCTAGTGTCTTCATATTCCCCCCTTCCGACGAGCAACTGAAAAGT
GGTACTGCTTCAGTAGTTTGCTTGTTGAACAACTTCTATCCACGCGAA
GCAAAAGTGCAATGGAAGGTCGATAACGCACTTCAATCTGGTAACTCT
CAAGAAAGTGTCACAGAGCAGGACAGTAAGGATAGTACATATAGCCTT
AGTTCAACACTGACTCTTTCTAAGGCCGATTATGAGAAACACAAGGTA
TACGCTTGTGAAGTGACCCACCAGGGACTTTCTAGCCCAGTTACCAAA
TCCTTCAACAGAGGAGAGTGC
SEQ ID NO: 28:
SynOA DVD2 Amino Acid Heavy Chain
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPEVQLVESGGGLVQPGGS
LRLSCAASGFTFSDAWMDWVRQAPGKGLEWVAEIRNKANNHARHYAES
VKGRFTISRDNAKNSLYLQMNSLRAEDTAVYYCARTYYYGSSYGYCDV
WGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNV
NHKPSNTKVDKKVEPKSCDKTHTCPPCPAPEAAGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGK
SEQ ID NO: 29
SynOA DVD2 DNA Heavy Chain
CAGGTACAGCTCCAGGAATCCGGGCCCGGTCTTGTGAAACCTTCAGAA
ACACTGTCTTTGACTTGTACAGTGAGTGGATTTTCTCTCATCGGGTAC
GATCTCAACTGGATCCGGCAACCTCCTGGGAAAGGTCTCGAATGGATA
GGGATCATCTGGGGCGATGGCACTACCGATTATAATTCAGCCGTGAAG
TCTCGAGTAACAATCTCCAAGGATACCAGTAAGAACCAGTTTAGTCTG
AAACTCAGCTCTGTTACAGCCGCCGACACAGCTGTCTACTATTGCGCC
AGGGGAGGGTATTGGTATGCTACCAGTTATTATTTCGACTATTGGGGA
CAGGGAACTTTGGTAACCGTCAGTAGTGCATCTACTAAAGGCCCCGAG
GTCCAGCTTGTTGAGTCTGGGGGTGGTCTCGTCCAACCTGGGGGTTCC
TTGCGGCTGTCATGTGCAGCTTCCGGCTTCACTTTTAGTGATGCTTGG
ATGGATTGGGTCCGGCAGGCCCCAGGTAAGGGTTTGGAATGGGTGGCC
GAAATAAGAAATAAGGCCAACAATCATGCTCGCCACTACGCAGAGTCC
GTAAAAGGACGATTTACAATCAGCCGTGACAACGCAAAAAATAGCTTG
TATCTTCAAATGAACAGTTTGAGGGCTGAGGATACAGCAGTGTATTAC
TGTGCTCGAACTTACTACTACGGGAGTAGTTATGGTTACTGTGATGTA
TGGGGTCAGGGAACTCTGGTAACCGTCTCAAGCGCTTCAACTAAAGGT
CCTTCAGTATTCCCCTTGGCTCCAAGCTCCAAGAGTACCAGTGGCGGT
ACCGCAGCTTTGGGCTGCCTGGTAAAAGATTATTTCCCAGAACCAGTA
ACTGTAAGCTGGAATAGCGGGGCTTTGACATCAGGGGTACACACCTTT
CCAGCTGTGCTTCAGTCTTCAGGCTTGTACAGTTTGTCTTCCGTAGTC
ACAGTCCCATCTTCTAGTCTGGGGACCCAAACCTATATCTGTAATGTC
AATCATAAGCCCTCAAATACAAAAGTGGACAAAAAAGTAGAGCCTAAG
AGCTGTGATAAAACACATACATGCCCTCCTTGTCCTGCCCCCGAAGCC
GCCGGCGGTCCCTCAGTATTTCTTTTTCCACCAAAACCCAAAGATACC
CTCATGATCAGTCGCACCCCAGAAGTCACCTGTGTCGTGGTAGATGTA
AGCCACGAGGATCCCGAGGTCAAATTCAACTGGTATGTGGATGGCGTA
GAAGTTCACAACGCCAAGACAAAGCCCAGGGAAGAACAGTACAATTCA
ACCTACAGGGTTGTCTCTGTACTGACCGTCCTTCACCAAGACTGGTTG
AACGGTAAAGAGTATAAATGTAAGGTATCCAACAAAGCTTTGCCAGCA
CCTATAGAGAAAACCATCTCCAAAGCTAAGGGTCAACCCAGAGAGCCA
CAAGTGTATACCCTGCCTCCCAGTAGAGATGAACTGACAAAGAACCAA
GTTTCTCTGACTTGCTTGGTCAAAGGGTTCTATCCTAGCGACATCGCT
GTAGAATGGGAATCAAATGGACAACCTGAAAATAACTACAAGACTACC
CCACCCGTGCTTGACAGTGATGGCAGTTTTTTCCTTTACAGTAAGCTC
ACTGTCGACAAGAGCCGATGGCAGCAAGGCAACGTCTTCTCTTGTTCC
GTTATGCATGAGGCCCTCCACAACCATTATACCCAAAAGAGTTTGTCA
TTGTCCCCTGGTAAA
SEQ ID NO: 30:
SynOA DVD2 Amino Acid Light Chain
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPDIQMTQSPSSLSASVGDRVTITCRTSENIYS
YLAWYQQKPGKAPKLLIYNAKTLAEGVPSRFSGSGSGTDFTLTISSLQ
PEDFATYYCQHHYGTPWTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 31:
SynOA DVD2 DNA Light Chain
GACATCCAGATGACACAGTCACCATCCTCTCTTAGTGCAAGTGTGGGC
GATCGGGTTACAATAACTTGTAGAGCCTCTCAATCAATTTCAAACAAT
CTGAATTGGTATCAACAGAAGCCTGGCAAGGCACCAAAACTTTTGATC
TACTACACTTCACGTTTCCATTCTGGTGTACCATCTCGCTTCTCCGGA
AGTGGAAGTGGCACAGATTTCACATTTACCATTAGTTCCTTGCAGCCC
GAAGATATAGCCACTTACTATTGTCAGCAGGAACATACCCTGCCATAC
ACTTTCGGCCAGGGCACAAAACTCGAGATCAAGCGTACTGTGGCAGCC
CCTGATATTCAGATGACCCAGTCCCCAAGTTCACTCTCAGCTTCTGTC
GGCGACCGTGTAACAATAACCTGCCGTACCTCTGAAAACATATACTCA
TACTTGGCCTGGTATCAACAAAAACCAGGTAAAGCTCCTAAGTTGCTC
ATCTATAATGCTAAGACTTTGGCTGAAGGAGTGCCCTCTCGATTCTCT
GGGTCAGGATCCGGAACAGATTTCACACTGACTATATCCAGCTTGCAA
CCAGAAGACTTCGCCACTTATTACTGCCAACATCACTATGGGACACCC
TGGACATTTGGGCAAGGAACCAAACTGGAGATAAAGCGGACTGTGGCC
GCACCTTCCGTTTTCATCTTTCCACCCTCTGACGAACAACTCAAATCA
GGGACTGCTTCAGTCGTGTGTCTCCTCAATAATTTTTATCCTCGGGAG
GCTAAAGTCCAATGGAAAGTAGACAATGCCCTCCAATCCGGGAATAGT
CAGGAAAGCGTCACAGAGCAAGATTCAAAAGACAGTACCTACTCATTG
TCTAGTACCCTTACCCTTAGTAAAGCAGACTACGAGAAACATAAGGTT
TATGCCTGCGAGGTGACTCACCAGGGTTTGTCTAGCCCAGTGACTAAA
TCCTTCAATCGCGGGGAGTGT
SEQ ID NO: 32:
SynOA BF1-Amino Acid Heavy Chain
7B4 with C-Term Tanezumab scFv
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSRDELTKNQVSLTCLVKGFYPSDI
AVEWESNGQPENNYKTTPPVLDSDGSFFLYSKLTVDKSRWQQGNVFSC
SVMHEALHNHYTQKSLSLSPGKGGGGSGGGGSGGGGSQVQLQESGPGL
VKPSETLSLTCTVSGFSLIGYDLNWIRQPPGKGLEWIGIIWGDGTTDY
NSAVKSRVTISKDTSKNQFSLKLSSVTAADTAVYYCARGGYWYATSYY
FDYWGQGTLVTVSSGGGGSGGGGSGGGGSDIQMTQSPSSLSASVGDRV
TITCRASQSISNNLNWYQQKPGKAPKLLIYYTSRFHSGVPSRFSGSGS
GTDFTFTISSLQPEDIATYYCQQEHTLPYTFGQGTKLEIK
SEQ ID NO: 33:
SynOA BF1-DNA Heavy Chain
7B4 with C-Term Tanezumab scFv
GAGGTGCAGCTGGTGGAAAGCGGCGGGGGGCTGGTGCAGCCCGGAGGC
TCACTGAGACTGTCTTGCGCTGCCAGCGGCTTCACCTTCAGCGACGCC
TGGATGGACTGGGTGAGGCAGGCCCCTGGGAAGGGGCTGGAGTGGGTG
GCTGAGATCCGGAATAAGGCCAATAACCACGCCAGGCACTATGCCGAG
AGTGTGAAAGGCAGATTTACCATCTCTAGGGACAATGCCAAGAACTCA
CTGTACCTGCAGATGAACTCCCTGAGAGCCGAGGACACCGCCGTGTAC
TACTGTGCCAGAACCTATTACTACGGAAGCAGCTACGGCTATTGCGAT
GTGTGGGGACAGGGAACCCTGGTGACTGTGTCTTCCTGAGCCTCCACC
AAGGGCCCCTCCGTGTTCCCACTGGCCCCGAGTAGCAAGTCTACCTCC
GGCGGGACAGCCGCACTGGGCTGCCTGGTGAAGGACTACTTTCCCGAG
CCTGTGACAGTGAGCTGGAATTCCGGGGCCCTGACATCAGGCGTGCAC
ACCTTCCCAGCCGTGCTCCAGAGCTCTGGCCTGTATAGTCTGAGCAGT
GTGGTGACCGTGCCCTCCAGCTCCCTGGGAACTCAGACCTATATCTGT
AACGTGAACCACAAGCCTTCTAATACCAAGGTGGATAAGAAAGTGGAA
CCAAAGTCATGCGACAAAACACACACCTGCCCCCCCTGCCCCGCCCCT
GAAGCCGCCGGCGGGCCTAGCGTGTTTCTGTTCCCTCCTAAGCCTAAG
GACACACTGATGATTTCCAGGACCCCCGAGGTGACCTGCGTCGTCGTG
GACGTGAGCCACGAGGACCCCGAGGTGAAATTCAACTGGTACGTGGAC
GGCGTGGAAGTGCACAACGCTAAAACCAAGCCTAGAGAGGAACAGTAC
AACTCTACATACAGGGTGGTGTCCGTCTTGACTGTGCTGCACCAGGAC
TGGCTGAACGGCAAGGAATATAAGTGCAAGGTGAGCAATAAAGCACTC
CCCGCCCCCATTGAAAAAACCATCAGCAAGGCTAAGGGCCAGCCTAGG
GAACCACAGGTGTATACCCTGCCACCTAGCAGAGACGAGCTGACCAAG
AATCAGGTGTCTCTGACATGTCTGGTGAAGGGGTTTTACCCTAGCGAC
ATTGCCGTCGAGTGGGAGTCTAATGGCCAGCCTGAGAACAATTATAAG
ACTACCCCTCCTGTGCTGGACAGTGATGGCTCATTTTTTCTGTACTCT
AAGCTGACAGTGGACAAGAGCAGGTGGCAGCAGGGCAATGTGTTCAGC
TGCAGCGTGATGCACGAGGCCCTGCATAATCATTATACCCAGAAGTCT
CTGTCTCTCAGCCCAGGCAAAGGCGGAGGGGGGTCTGGCGGCGGGGGT
TCCGGCGGCGGGGGCTCCCAGGTGCAGCTGCAGGAGAGCGGACCTGGC
CTGGTGAAACCCAGCGAGACCCTGTCCCTGACATGCACCGTGAGCGGC
TTCTCCCTGATCGGGTATGACCTGAATTGGATCCGGCAGCCTCCTGGC
AAGGGTCTGGAGTGGATCGGAATCATCTGGGGCGACGGCACCACCGAT
TATAACAGCGCCGTGAAGTCTCGGGTGACTATCTCTAAGGATACTTCC
AAGAACCAGTTTTCCCTGAAGCTGAGCTCTGTGACCGCTGCCGATACC
GCCGTGTACTACTGCGCCAGGGGCGGTTACTGGTACGCCACCAGCTAC
TACTTCGACTACTGGGGCCAGGGCACCCTGGTGACCGTGTCCAGCGGC
GGCGGCGGCTCTGGCGGTGGGGGCTCCGGCGGCGGAGGGTCTGACATT
CAGATGACCCAGAGCCCCTCCTCCCTGTCTGCCTCCGTGGGCGACAGA
GTGACCATCACATGTAGAGCCTCCCAGTCAATATCTAACAACCTGAAT
TGGTATCAGCAGAAGCCCGGCAAAGCCCCTAAGCTGCTGATTTACTAC
ACTTCTCGCTTTCACTCCGGAGTGCCCAGCAGATTTAGTGGGAGCGGC
TCTGGGACAGACTTCACTTTTACCATCAGTAGCCTGCAGCCCGAGGAC
ATCGCCACCTATTACTGCCAGCAGGAGCATACCCTGCCCTACACTTTC
GGACAGGGGACCAAGCTGGAGATCAAGTGA
Additional Constructs
SEQ ID NO: 34:
SynOA DVD3 Amino Acid Heavy Chain
EVQLVESGGGLVQPGGSLRLSCAASGFTFSDAWMDWVRQAPGKGLEWV
AEIRHKANDHAIFYDESVKGRFTISRDDSKNTVYLQMNSLRAEDTAVY
YCTSPFAYWGQGTLVTVSSASTKGPQVQLQESGPGLVKPSETLSLTCT
VSGFSLIGYDLNWIRQPPGKGLEWIGIIWGDGTTDYNSAVKSRVTISK
DTSKNQFSLKLSSVTAADTAVYYCARGGYWYATSYYFDYWGQGTLVTV
SSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKV
DKKVEPKSCDKTHTCPPCPAPEAAGGPSVFLFPPKPKDTLMISRTPEV
TCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSR
DELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 35:
SynOA DVD3 Amino Acid Light Chain
DIQMTQSPSSLSASVGDRVTITCKASQSVGTTIVWYQQKPGKAPKLLI
YSASNRHTGVPSRFSGSGSGTDFTLTISSLQPEDFATYYCQQYTSYPF
TFGQGTKLEIKRTVAAPDIQMTQSPSSLSASVGDRVTITCRASQSISN
NLNWYQQKPGKAPKLLIYYTSRFHSGVPSRFSGSGSGTDFTFTISSLQ
PEDIATYYCQQEHTLPYTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 36:
SynOA DVD4 Amino Acid Heavy Chain
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPEVQLVESGGGLVQPGGS
LRLSCAASGFTFSDAWMDWVRQAPGKGLEWVAEIRHKANDHAIFYDES
VKGRFTISRDDSKNTVYLQMNSLRAEDTAVYYCTSPFAYWGQGTLVTV
SSASTKGPASTKGPSVFPLAPSSKSTSGGTAALGCLVKDYFPEPVTVS
WNSGALTSGVHTFPAVLQSSGLYSLSSVVTVPSSSLGTQTYICNVNHK
PSNTKVDKKVEPKSCDKTHTCPPCPAPEAAGGPSVFLFPPKPKDTLMI
SRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVY
TLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSP
GK
SEQ ID NO: 37:
SynOA DVD4 Amino Acid Light Chain
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPDIQMTQSPSSLSASVGDRVTITCKASQSVGT
TIVWYQQKPGKAPKLLIYSASNRHTGVPSRFSGSGSGTDFTLTISSLQ
PEDFATYYCQQYTSYPFTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 38:
SynOA CM2-Amino Acid 12F4 Heavy Chain
(with AA and KIH mutations):
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVCTLPPSRDELTKNQVSLSCAVKGFYPSDIAVEWESNGQP
ENNYKTTPPVLDSDGSFFLVSKLTVDKSRWQQGNVFSCSVMHEALHNH
YTQKSLSLSPGK
SEQ ID NO: 39:
SynOA CM2-Amino Acid 12F4 Light Chain
DIQMTQSPSSLSASVGDRVTITCKASQSVGTTIVWTQQLPGKAPKLLI
YSASNRHTGVPSRFSGSGSGTDFTLTISSLQPEDFATYYCQQYTSYPF
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 40
SynOA BF4-Amino Acid 12F4 Heavy Chain
AA mutant + Tanezumab scFv
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLSCLVKGFYPSDIAVEWESNGQP
ENNYKTTPPVLDSDGSFFLYSKLTVDKSRWQQGNVFSCSVMHEALHNH
YTQKSLSLSPGKGGGGSGGGGSGGGGSQVQLQESGPGLVKPSETLSLT
CTVSGFSLIGYDLNWIRQPPGKGLEWIGIIWGDGTTDYNSAVKSRVTI
SKDTSKNQFSLKLSSVTAADTAVYYCARGGYWYATSYYFDYWGQGTLV
TVSSGGGGSGGGGSGGGGSDIQMTQSPSSLSASVGDRVTITCRASQSI
SNNLNWYQQKPGKAPKLLIYYTSRFHSGVPSRFSGSGSGTDFTFTISS
LQPEDIATYYCQQEHTLPYTFGQGTKLEIK
SEQ ID NO: 41:
SynOA DVD9 Amino Acid 7B4 +
Tanezumab Heavy Chain
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPQVQLQES
GPGLVKPSETLSLTCTVSGFSLIGYDLNWIRQPPGKGLEWIGIIWGDG
TTDYNSAVKSRVTISKDTSKNQFSLKLSSVTAADTAVYYCARGGYWYA
TSYYFDYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNH
YTQKSLSLSPGK
SEQ ID NO: 42:
SynOA DVD9 Amino Acid 7B4 +
Tanezumab Light Chain
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPSVFIFPPDIQMTQSPSSLSASVGDRVTITCR
ASQSISNNLNWYQQKPGKAPKLLIYYTSRFHSGVPSRFSGSGSGTDFT
FTISSLQPEDIATYYCQQEHTLPYTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 43:
SynOA DVD10-Amino Acid Tanezumab +
7B4 Heavy Chain
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPSVFPLAPEVQLVESGGG
LVQPGGSLRLSCAASGFTFSDAWMDWVRQAPGKGLEWVAEIRNKANNH
ARHYAESVKGRFTISRDNAKNSLYLQMNSLRAEDTAVYYCARTYYYGS
SYGYCDVWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNH
YTQKSLSLSPGK
SEQ ID NO: 44:
SynOA DVD10-Amino Acid Tanezumab +
7B4 Light Chain
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPSVFIFPPDIQMTQSPSSLSASVGDRVTITCR
TSENIYSYLAWYQQKPGKAPKLLIYNAKTLAEGVPSRFSGSGSGTDFT
LTISSLQPEDFATYYCQHHYGTPWTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 45:
SynOA DVD11 Amino Acid 12F4 +
Tanezumab Heavy Chain
EVQLVESGGGLVQPGGSLRLSCAASGFTFSDAWMDWVRQAPGKGLEWV
AEIRHKANDHAIFYDESVKGRFTISRDDSKNTVYLQMNSLRAEDTAVY
YCTSPFAYWGQGTLVTVSSASTKGPSVFPLAPQVQLQESGPGLVKPSE
TLSLTCTVSGFSLIGYDLNWIRQPPGKGLEWIGIIWGDGTTDYNSAVK
SRVTISKDTSKNQFSLKLSSVTAADTAVYYCARGGYWYATSYYFDYWG
QGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNH
KPSNTKVDKKVEPKSCDKTHTCPPCPAPEAAGGPSVFLFPPKPKDTLM
ISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQV
YTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLS
PGK
SEQ ID NO: 46:
SynOA DVD11 Amino Acid 12F4 +
Tanezumab Light Chain
DIQMTQSPSSLSASVGDRVTITCKASQSVGTTIVWYQQKPGKAPKLLIY
SASNRHTGVPSRFSGSGSGTDFTLTISSLQPEDFATYYCQQYTSYPFT
FGQGTKLEIKRTVAAPSVFIFPPDIQMTQSPSSLSASVGDRVTITCRA
SQSISNNLNWYQQKPGKAPKLLIYYTSRFHSGVPSRFSGSGSGTDFTF
TISSLQPEDIATYYCQQEHTLPYTFGQGTKLEIKRTVAAPSVFIFPPS
DEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID NO: 47:
SynOA DVD12 Amino Acid Tanezumab +
12F4 Heavy Chain
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPSVFPLAPEVQLVESGGG
LVQPGGSLRLSCAASGFTFSDAWMDWVRQAPGKGLEWVAEIRHKANDH
AIFYDESVKGRFTISRDDSKNTVYLQMNSLRAEDTAVYYCTSPFAYWG
QGTLVTVSSASTKGPASTKGPSVFPLAPSSKSTSGGTAALGCLVKDYF
PEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQ
KSLSLSPGK
SEQ ID NO: 48
SynOA DVD12 Amino Acid Tanezumab +
12F4 Light Chain
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPSVFIFPPDIQMTQSPSSLSASVGDRVTITCK
ASQSVGTTIVWYQQKPGKAPKLLIYSASNRHTGVPSRFSGSGSGTDFT
LTISSLQPEDFATYYCQQYTSYPFTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 49:
ADAMTS 5 (CRB0017) HC with AA and
KIH mutations
EVQLLESGGGLVQPGGSLRLSCAASGFTFSSYWMHWVRQAPGKGLEWV
AYINPSTGYTEYNQKFKRFTISRDSKNTVYLQMNSLRAEDTAVYYCTS
GGYDDLGYWGQGTLVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVCTLPPSRDELTKNQVSLSCAVKGFYPSDIAVEWESNGQ
PENNYKTTPPVLDSDGSFFLVSKLTVDKSRWQQGNVFSCSVMHEALHN
HYTQKSLSLSPGK
SEQ ID NO: 50:
SynOA CM8-Amino Acid p75NTR Fc Heavy Chain
w AA and KIH mutations
KEACPTGLYTHSGECCKACNLGEGVAQPCGANQTVCEPCLDSVTFSDV
VSATEPCKPCTECVGLQSMSAPCVEADDAVCRCAYGYYQDETTGRCEA
CRVCEAGSGLVFSCQDKQNTVCEECPDGTYSDEANHVDPCLPCTVCED
TERQLRECTRWADAECEEIPGRWITRSTPPEGSDSTAPSTQEPEAPPE
QDLIASTVAGVVTTVMGSSQPVVTRGTTDNDIEGRMDPKSCDKTHTCP
PCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKV
SNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQ
GNVFSCSVMHEALHNHYTQKSLSLSPGK
SEQ ID NO: 51:
SynOA CM9-Amino Acid TrkA Fc Heavy Chain
w AA and KIH mutations
AAPCPDACCPHGSSGLRCTRDGALDSLHHLPGAENLTELYIENQQHLQ
HLELRDLRGLGELRNLTIVKSGLRFVAPDAFHFTPRLSRLNLSFNALE
SLSWKTVQGLSLQELVLSGNPLHCSCALRWLQRWEEEGLGGVPEQKLQ
CHGQGPLAHMPNASCGVPTLKVQVPNASVDVGDDVLLRCQVEGRGLEQ
AGWILTELEQSATVMKSGGLPSLGLTLANVTSDLNRKNVTCWAENDVG
RAEVSVQVNVSFPASVQLHTAVEMHHWCIPFSVDGQPAPSLRWLFNGS
VLNETSFIFTEFLEPAANETVRHGCLRLNQPTHVNNGNYTLLAANPFG
QASASIMAAFMDNPFEFNPEDPIPVSFSPVDTNSTSGDPVEIEGRIDP
KSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDW
LNGKEYKCKVSNKALPAPIEKTISKAKGQPREPQVYTLPPCRDELTKN
QVSLWCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 52:
SynOA DM1-Amino Acid 7B4 Heavy Chain
with AA and Duobody (F405L) mutation
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSRDELTKNQVSLSCLVKGFYPSDI
AVEWESNGQPENNYKTTPPVLDSDGSFLLYSKLTVDKSRWQQGNVFSC
SVMHEALHNHYTQKSLSLSPGK
SEQ ID NO: 53:
SynOA DM1-Amino Acid Tanezumab Heavy Chain
w AA and Duobody (K409R) Mutant
QVQLQESGPGLVKPSE
TLSLTCTVSGFSLIGYDLNWIRQPPGKGLEWIGIIWGDGTTDYNSAVK
SRVTISKDTSKNQFSLKLSSVTAADTAVYYCARGGYWYATSYYFDYWG
QGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNH
KPSNTKVDKKVEPKSCDKTHTCPPCPAPEAAGGPSVFLFPPKPKDTLM
ISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQV
YTLPPSRDELTKNQVSLTCLVKGFYPSDIAVEWESNGQPENNYKTTPP
VLDSDGSFFLYSRLTVDKSRWQQGNVFSCSVMHEALHNHYTQKSLSLS
PGK
SEQ ID NO: 54:
SynOA DM1-Amino Acid Tanezumab Light Chain
w AA and Duobody (K409R) Mutant
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 55:
SynOA DM3-Amino Acid 12F4 Heavy Chain
with AA and Duobody (F405L) Mutants
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLSCLVKGFYPSDIAVEWESNGQP
ENNYKTTPPVLDSDGSFLLYSKLTVDKSRWQQGNVFSCSVMHEALHNH
YTQKSLSLSPGK
SEQ ID NO: 56:
SynOA CM5-Amino Acid NGF (Fasinumab) Heavy Chain
w AA and KIH mutations
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASVAAPSVFIFPPSDEQLKSGTASVV
CLLNNFYPREAKVQWKVDNALQSGNSQESVTEQDSKDSTYSLSSTLTL
SKADYEKHKVYACEVTHQGLSSPVTKSFNRGECDKTHTCPPCPAPEAA
GGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAP
IEKTISKAKGQPREPQVYTLPPCRDELTKNQVSLWCLVKGFYPSDIAV
EWESNGQPENNYKTTPPVLDSDGSFFLYSKLTVDKSRWQQGNVFSCSV
MHEALHNHYTQKSLSLSPGK
SEQ ID NO: 57:
SynOA CM5-Amino Acid NGF (Fasinumab) Light Chain
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKVTVLSSASTKGPSVFPLAPSSKSTSGGTAALGCLVKDYFPE
PVTVSWNSGALTSGVHTFPAVLQSSGLYSLSSVVTVPSSSLGTQTYIC
NVNHKPSNTKVDKKVEPKSC
SEQ ID NO: 58:
SynOA DVD17-Amino Acid 7B4 +
Fasinumab DVD Heavy Chain w SS and AA mutant
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPQVQLVQSGAEVKKP
GASVKVSCKVSGFTLTELSIHWVRQAPGKGLEWMGGFDPEDGETIYAQ
KFQGRVTMTEDTSTDTAYMELTSLRSEDTAVYYCSTIGVVTNFDNWGQ
GTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHK
PSNTKVDKKVEPKSCDKTHTCPPCPAPEAAGGPSVFLFPPKPKDTLMI
SRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVY
TLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSP
GK
SEQ ID NO: 59:
SynOA DVD17-Amino Acid 7B4 +
Fasinumab DVD Light Chain w SS and AA mutant
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPDIQMTQSPSSLSASAGDRVTITCRASQAIRN
DLGWYQQKPGKAPKRLIYAAFNLQSGVPSRFSGSGSGTEFTLTISSLQ
PEDLASYYCQQYNRYPWTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 60:
SynOA DVD18-Amino Acid 12F4 +
Fasinumab DVD Heavy Chain w SS and AA mutant
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPQVQLVQSGAEVKKPGASVKVSCKV
SGFTLTELSIHWVRQAPGKGLEWMGGFDPEDGETIYAQKFQGRVTMTE
DTSTDTAYMELTSLRSEDTAVYYCSTIGVVTNFDNWGQGTLVTVSSAS
TKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVV
VDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELT
KNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 61:
SynOA DVD18-Amino Acid 12F4 +
Fasinumab DVD Light Chain w SS and AA mutant
DIQMTQSPSSLSASVGDRVTITCKASQSVGTTIVWTQQLPGLAPKLLI
YSASNRHTGVPSRFSGSGSGTDFTLTISSLQPEDFATYYCQQYTSYPF
TFGQGTKEIKRTVAAPDIQMTQSPSSLSASAGDRVTITCRASQAIRND
LGWYQQKPGKAPKRLIYAAFNLQSGVPSRFSGSGSGTEFTLTISSLQP
EDLASYYCQQYNRYPWTFGQGTKLEIKRTVAAPSVFIFPPSDEQLKSG
TASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 62:
SynOA DVD19-Amino Acid Fasinumab +
7B4 DVD Heavy Chain w SS and AA mutant
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPEVQLVESGGGLVQPGGSLRL
SCAASGFTFSDAWMDWVRQAPGKGLEWVAEIRNKANNHARHYAESVKG
RFTISRDNAKNSLYLQMNSLRAEDTAVYYCARTYYYGSSYGYCDVWGQ
GTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHK
PSNTKVDKKVEPKSCDKTHTCPPCPAPEAAGGPSVFLFPPKPKDTLMI
SRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVY
TLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSP
GK
SEQ ID NO: 63:
SynOA DVD19-Amino Acid Fasinumab +
7B4 DVD Light Chain w SS and AA mutant
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKLEIKRTVAAPDIQMTQSPSSLSASVGDRVTITCRTSENIYS
YLAWYQQKPGKAPKLLIYNAKTLAEGVPSRFSGSGSGTDFTLTISSLQ
PEDFATYYCQHHYGTPWTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 64:
SynOA DVD20-Amino Acid Fasinumab +
12F4 DVD Heavy Chain w SS and AA mutant
QVQLVQSGAEVKKPGASVKVSCKVSGFTLTELSIHWVRQAPGKGLEW
MGGFDPEDGETIYAQKFQGRVT
MTEDTSTDTAYMELTSLRSEDTAVYYCSTIGVVTNFDNWGQGTLVTVS
SASTKGPEVQLLESGGGLVQPGGSLRLSCAASGFTFSDAWMDWVRQAP
GKGLEWVAEIRHKANDHAIFYDESVKGRFTISRDSKNTVYLQMNSLRA
EDTAVYYCTSPFAYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVM
HEALHNHYTQKSLSLSPGK
SEQ ID NO: 65:
SynOA DVD20-Amino Acid Fasinumab +
12F4 DVD Light Chain w SS and AA mutant
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKLEIKRTVAAPDIQMTQSPSSLSASVGDRVTITCKASQSVGT
TIVWTQQLPGLAPKLLIYSASNRHTGVPSRFSGSGSGTDFTLTISSLQ
PEDFATYYCQQYTSYPFTFGQGTKEIKRTVAAPSVFIFPPSDEQLKSG
TASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 66:
SynOA DVD21-Amino Acid 7B4 +
Fasinumab DVD Heavy Chain
w LL and AA mutant
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPQVQLVQS
GAEVKKPGASVKVSCKVSGFTLTELSIHWVRQAPGKGLEWMGGFDPED
GETIYAQKFQGRVTMTEDTSTDTAYMELTSLRSEDTAVYYCSTIGVVT
NFDNWGQGTLVTVSSASTKGPSVFPLAPSSKSTSGGTAALGCLVKDYF
PEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQ
KSLSLSPGK
SEQ ID NO: 67:
SynOA DVD21-Amino Acid 7B4 +
Fasinumab DVD Light Chain w LL and AA mutant
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPSVFIFPPDIQMTQSPSSLSASAGDRVTITCR
ASQAIRNDLGWYQQKPGKAPKRLIYAAFNLQSGVPSRFSGSGSGTEFT
LTISSLQPEDLASYYCQQYNRYPWTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 68:
SynOA DVD22-Amino Acid 12F4 +
Fasinumab DVD Heavy Chain w LL and AA mutant
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPQVQLVQSGAEVKKPGAS
VKVSCKVSGFTLTELSIHWVRQAPGKGLEWMGGFDPEDGETIYAQKFQ
GRVTMTEDTSTDTAYMELTSLRSEDTAVYYCSTIGVVTNFDNWGQGTL
VTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSN
TKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 69:
SynOA DVD22-Amino Acid 12F4 +
Fasinumab DVD Light Chain w LL and AA mutant
DIQMTQSPSSLSASVGDRVTITCKASQSVGTTIVWTQQLPGLAPKLLI
YSASNRHTGVPSRFSGSGSGTDFTLTISSLQPEDFATYYCQQYTSYPF
TFGQGTKEIKRTVAAPSVFIFPPDIQMTQSPSSLSASAGDRVTITCRA
SQAIRNDLGWYQQKPGKAPKRLIYAAFNLQSGVPSRFSGSGSGTEFTL
TISSLQPEDLASYYCQQYNRYPWTFGQGTKLEIKRTVAAPSVFIFPPS
DEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID NO: 70:
SynOA DVD23-Amino Acid Fasinumab +
7B4 DVD Heavy Chain w LL and AA mutant
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPSVFPLAPEVQLVESGGGLVQ
PGGSLRLSCAASGFTFSDAWMDWVRQAPGKGLEWVAEIRNKANNHARH
YAESVKGRFTISRDNAKNSLYLQMNSLRAEDTAVYYCARTYYYGSSYG
YCDVWGQGTLVTVSSASTKGPSVFPLAPSSKSTSGGTAALGCLVKDYF
PEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPK
PKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQ
KSLSLSPGK
SEQ ID NO: 71:
SynOA DVD23-Amino Acid Fasinumab +
7B4 DVD Light Chain w LL and AA mutant
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKLEIKRTVAAPSVFIFPPDIQMTQSPSSLSASVGDRVTITCR
TSENIYSYLAWYQQKPGKAPKLLIYNAKTLAEGVPSRFSGSGSGTDFT
LTISSLQPEDFATYYCQHHYGTPWTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 72:
SynOA DVD24-Amino Acid Fasinumab +
12F4 DVD Heavy Chain w LL and AA mutant
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPSVFPLAPEVQLLESGGGLVQ
PGGSLRLSCAASGFTFSDAWMDWVRQAPGKGLEWVAEIRHKANDHAIF
YDESVKGRFTISRDSKNTVYLQMNSLRAEDTAVYYCTSPFAYWGQGTL
VTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSN
TKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 73:
SynOA DVD24-Amino Acid Fasinumab +
12F4 DVD Light Chain w LL and AA mutant
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKLEIKRTVAAPSVFIFPPDIQMTQSPSSLSASVGDRVTITCK
ASQSVGTTIVWTQQLPGLAPKLLIYSASNRHTGVPSRFSGSGSGTDFT
LTISSLQPEDFATYYCQQYTSYPFTFGQGTKEIKRTVAAPSVFIFPPS
DEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID NO: 74:
SynOA DVD25-Amino Acid CRB0017 +
Tanezumab DVD Heavy Chain w LL and AA mutant
EVQLVESGGGLVQPGGSLRLSCAASGFTFSSYWMHWVRQAPGKGLEWV
AYINPSTGYTEYNQKFKRFTISRDDSKNTVYLQMNSLRAEDTAVYYCT
SGGYDDLGYWGQGTLVTVSSASTKGPQVQLQESGPGLVKPSETLSLTC
TVSGFSLIGYDLNWIRQPPGKGLEWIGIIWGDGTTDYNSAVKSRVTIS
KDTSKNQFSLKLSSVTAADTAVYYCARGGYWYATSYYFDYWGQGTLVT
VSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTK
VDKKVEPKSCDKTHTCPPCPAPEAAGGPSVFLEPPKPKDTLMISRTPE
VTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPS
RDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 75:
SynOA DVD25-Amino Acid CRB0017 +
Tanezumab DVD Light Chain w LL and AA mutant
DIQMTQSPSSLSASVGDRVTITCRSSKSLLYKDGKTYLYWYQQKPGKA
PKLLIYLMSTRASGVPSRFSGSGSGTDFTLTISSLQPEDFATYYCQQL
VUYPYTFGQGTKLEIKRTVAAPDIQMTQSPSSLSASVGDRVTITCRAS
QSISNNLNWYQQKPGKAPKLLIYYTSRFHSGVPSRFSGSGSGTDFTFT
ISSLQPEDIATYYCQQEHTLPYTFGQGTKLEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 76:
SynOA DVD26-Amino Acid CRB0017 +
Tanezumab DVD Heavy Chain w LL and AA mutant
EVQLVESGGGLVQPGGSLRLSCAASGFTFSSYWMHWVRQAPGKGLEWV
AYINPSTGYTEYNQKFKRFTISRDDSKNTVYLQMNSLRAEDTAVYYCT
SGGYDDLGYWGQGTLVTVSSASTKGPQVQLVQSGAEVKKPGASVKVSC
KVSGFTLTELSIHWVRQAPGKGLEWMGGFDPEDGETIYAQKFQGRVTM
TEDTSTDTAYMELTSLRSEDTAVYYCSTIGVVTNFDNWGQGTLVTVSS
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 77:
SynOA DVD26-Amino Acid CRB0017 +
Tanezumab DVD Light Chain w LL and AA mutant
DIQMTQSPSSLSASVGDRVTITCRSSKSLLYKDGKTYLYWYQQKPGKA
PKLLIYLMSTRASGVPSRFSGSGSGTDFTLTISSLQPEDFATYYCQQL
VUYPYTFGQGTKLEIKRTVAAPDIQMTQSPSSLSASVGDRVTITCKAS
QSVGTTIVWTQQLPGLAPKLLIYSASNRHTGVPSRFSGSGSGTDFTLT
ISSLQPEDFATYYCQQYTSYPFTFGQGTKEIKRTVAAPSVFIFPPSDE
QLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 78:
SynOA DVD27-Amino Acid CRB0017 +
Fulranumab DVD Heavy Chain w LL and AA mutant
EVQLVESGGGLVQPGGSLRLSCAASGFTFSSYWMHWVRQAPGKGLEWV
AYINPSTGYTEYNQKFKRFTISRDDSKNTVYLQMNSLRAEDTAVYYCT
SGGYDDLGYWGQGTLVTVSSASTKGPEVQLVESGGGLVQPGGSLRLSC
AASGFTLRSYSMNWVRQAPGKGLEWVSYISRSSHTIFYADSVKGRFTI
SRDNAKNSLYLQMDSLRDEDTAMYYCARVYSSGWHVSDYFDYWGQGIL
VTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSN
TKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 79:
SynOA DVD27-Amino Acid CRB0017 +
Fulranumab DVD Light Chain w LL and AA mutant
DIQMTQSPSSLSASVGDRVTITCRSSKSLLYKDGKTYLYWYQQKPGKA
PKLLIYLMSTRASGVPSRFSGSGSGTDFTLTISSLQPEDFATYYCQQL
VUYPYTFGQGTKLEIKRTVAAPAIQLTQSPSSLSASVGDRVTITCRAS
QGISSALAWYQQKPGKAPKLLIYDASSLESGVPSRFSGSGSGTDFTLT
ISSLQPEDFATYYCQQFNSYPLTFGGGTKLEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 80:
SynOA DVD28-Amino Acid Tanezumab +
CRB0017 DVD Heavy Chain w LL and AA mutant
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPEVQLVESGGGLVQPGGSLRL
SCAASGFTFSSYWMHWVRQAPGKGLEWVAYINPSTGYTEYNQKFKRFT
ISRDDSKNTVYLQMNSLRAEDTAVYYCTSGGYDDLGYWGQGTLVTVSS
ASTKGPASTKGPSVFPLAPSSKSTSGGTAALGCLVKDYFPEPVTVSWN
SGALTSGVHTFPAVLQSSGLYSLSSVVTVPSSSLGTQTYICNVNHKPS
NTKVDKKVEPKSCDKTHTCPPCPAPEAAGGPSVFLFPPKPKDTLMISR
TPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTL
PPSRDELTKNQVSLTCLVKGFYPSDIAVEWESNGQPENNYKTTPPVLD
SDGSFFLYSKLTVDKSRWQQGNVFSCSVMHEALHNHYTQKSLSL
SEQ ID NO: 81:
SynOA DVD28-Amino Acid Tanezumab +
CRB0017 DVD Light Chain w LL and AA mutant
DIQMTQSPSSLSASVGDRVTITCKASQSVGTTIVWTQQLPGLAPKLLI
YSASNRHTGVPSRFSGSGSGTDFTLTISSLQPEDFATYYCQQYTSYPF
TFGQGTKEIKRTVAAPDIQMTQSPSSLSASVGDRVTITCRSSKSLLYK
DGKTYLYWYQQKPGKAPKLLIYLMSTRASGVPSRFSGSGSGTDFTLTI
SSLQPEDFATYYCQQLVUYPYTFGQGTKLEIKRTVAAPSVFIFPPSDE
QLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 82:
SynOA DVD29-Amino Acid Fasinumab +
CRB0017 DVD Heavy Chain w LL and AA mutant
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPEVQLVESGGGLVQPG
GSLRLSCAASGFTFSSYWMHWVRQAPGKGLEWVAYINPSTGYTEYNQK
FKRFTISRDDSKNTVYLQMNSLRAEDTAVYYCTSGGYDDLGYWGQGTL
VTVSSASTKGPASTKGPSVFPLAPSSKSTSGGTAALGCLVKDYFPEPV
TVSWNSGALTSGVHTFPAVLQSSGLYSLSSVVTVPSSSLGTQTYICNV
NHKPSNTKVDKKVEPKSCDKTHTCPPCPAPEAAGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
L
SEQ ID NO: 83:
SynOA DVD29-Amino Acid Fasinumab +
CRB0017 DVD Light Chain w LL and AA mutant
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPDIQMTQSPSSLSASVGDRVTITCRSSKSLLY
KDGKTYLYWYQQKPGKAPKLLIYLMSTRASGVPSRFSGSGSGTDFTLT
ISSLQPEDFATYYCQQLVUYPYTFGQGTKLEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 84:
SynOA DVD30-Amino Acid Fulranumab +
CRB0017 DVD Heavy Chain w LL and AA mutant
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPEVQLVESGGGLVQPG
GSLRLSCAASGFTFSSYWMHWVRQAPGKGLEWVAYINPSTGYTEYNQK
FKRFTISRDDSKNTVYLQMNSLRAEDTAVYYCTSGGYDDLGYWGQGTL
VTVSSASTKGPASTKGPSVFPLAPSSKSTSGGTAALGCLVKDYFPEPV
TVSWNSGALTSGVHTFPAVLQSSGLYSLSSVVTVPSSSLGTQTYICNV
NHKPSNTKVDKKVEPKSCDKTHTCPPCPAPEAAGGPSVFLFPPKPKDT
LMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
L
SEQ ID NO: 85:
SynOA DVD30-Amino Acid Fulranumab +
CRB0017 DVD Light Chain w LL and AA mutant
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPDIQMTQSPSSLSASVGDRVTITCRSSKSLLY
KDGKTYLYWYQQKPGKAPKLLIYLMSTRASGVPSRFSGSGSGTDFTLT
ISSLQPEDFATYYCQQLVUYPYTFGQGTKLEIKRTVAAPSVFIFPPSD
EQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 86:
SynOA DVD31-Amino Acid CRB0017 +
Tanezumab DVD Heavy Chain
w LL and AA mutant
EVQLVESGGGLVQPGGSLRLSCAASGFTFSSYWMHWVRQAPGKGLEWV
AYINPSTGYTEYNQKFKRFTISRDDSKNTVYLQMNSLRAEDTAVYYCT
SGGYDDLGYWGQGTLVTVSSASTKGPSVFPLAPQVQLQESGPGLVKPS
ETLSLTCTVSGFSLIGYDLNWIRQPPGKGLEWIGIIWGDGTTDYNSAV
KSRVTISKDTSKNQFSLKLSSVTAADTAVYYCARGGYWYATSYYFDYW
GQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSL
SPGK
SEQ ID NO: 87:
SynOA DVD31-Amino Acid CRB0017 +
Tanezumab DVD Light Chain w LL and AA mutant
DIQMTQSPSSLSASVGDRVTITCRSSKSLLYKDGKTYLYWYQQKPGKA
PKLLIYLMSTRASGVPSRFSGSGSGTDFTLTISSLQPEDFATYYCQQL
VUYPYTFGQGTKLEIKRTVAAPSVFIFPPDIQMTQSPSSLSASVGDRV
TITCRASQSISNNLNWYQQKPGKAPKLLIYYTSRFHSGVPSRFSGSGS
GTDFTFTISSLQPEDIATYYCQQEHTLPYTFGQGTKLEIKRTVAAPSV
FIFPPSDEQLKSGTASVVCLLNNFYPREAKVQWKVDNALQSGNSQESV
TEQDSKDSTYSLSSTLTLSKADYEKHKVYACEVTHQGLSSPVTKSFNR
GEC
SEQ ID NO: 88:
SynOA DVD32-Amino Acid CRB0017 +
Fasinumab DVD Heavy Chain w LL and AA mutant
EVQLVESGGGLVQPGGSLRLSCAASGFTFSSYWMHWVRQAPGKGLEWV
AYINPSTGYTEYNQKFKRFTISRDDSKNTVYLQMNSLRAEDTAVYYCT
SGGYDDLGYWGQGTLVTVSSASTKGPSVFPLAPQVQLVQSGAEVKKPG
ASVKVSCKVSGFTLTELSIHWVRQAPGKGLEWMGGFDPEDGETIYAQK
FQGRVTMTEDTSTDTAYMELTSLRSEDTAVYYCSTIGVVTNFDNWGQG
TLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMIS
RTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYT
LPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
K
SEQ ID NO: 89:
SynOA DVD32-Amino Acid CRB0017 +
Fasinumab DVD Light Chain w LL and AA mutant
DIQMTQSPSSLSASVGDRVTITCRSSKSLLYKDGKTYLYWYQQKPGKA
PKLLIYLMSTRASGVPSRFSGSGSGTDFTLTISSLQPEDFATYYCQQL
VUYPYTFGQGTKLEIKRTVAAPSVFIFPPDIQMTQSPSSLSASVGDRV
TITCKASQSVGTTIVWTQQLPGLAPKLLIYSASNRHTGVPSRFSGSGS
GTDFTLTISSLQPEDFATYYCQQYTSYPFTFGQGTKEIKRTVAAPSVF
IFPPSDEQLKSGTASVVCLLNNFYPREAKVQWKVDNALQSGNSQESVT
EQDSKDSTYSLSSTLTLSKADYEKHKVYACEVTHQGLSSPVTKSFNRG
EC
SEQ ID NO: 90:
SynOA DVD33-Amino Acid CRB0017 +
Fulranumab DVD Heavy Chain w LL and AA mutant
EVQLVESGGGLVQPGGSLRLSCAASGFTFSSYWMHWVRQAPGKGLEWV
AYINPSTGYTEYNQKFKRFTISRDDSKNTVYLQMNSLRAEDTAVYYCT
SGGYDDLGYWGQGTLVTVSSASTKGPSVFPLAPEVQLVESGGGLVQPG
GSLRLSCAASGFTLRSYSMNWVRQAPGKGLEWVSYISRSSHTIFYADS
VKGRFTISRDNAKNSLYLQMDSLRDEDTAMYYCARVYSSGWHVSDYFD
YWGQGILVTVSSASTKGPSVFPLAPSSKSTSGGTAALGCLVKDYFPEP
VTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYN
STYRVVSVLTVLHQDWLNGKEYKCKVSNKALPAPIEKTISKAKGQPRE
PQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSL
SLSPGK
SEQ ID NO: 91:
SynOA DVD33-Amino Acid CRB0017 +
Fulranumab DVD Light Chain w LL and AA mutant
DIQMTQSPSSLSASVGDRVTITCRSSKSLLYKDGKTYLYWYQQKPGKA
PKLLIYLMSTRASGVPSRFSGSGSGTDFTLTISSLQPEDFATYYCQQL
VUYPYTFGQGTKLEIKRTVAAPSVFIFPPAIQLTQSPSSLSASVGDRV
TITCRASQGISSALAWYQQKPGKAPKLLIYDASSLESGVPSRFSGSGS
GTDFTLTISSLQPEDFATYYCQQFNSYPLTFGGGTKLEIKRTVAAPSV
FIFPPSDEQLKSGTASVVCLLNNFYPREAKVQWKVDNALQSGNSQESV
TEQDSKDSTYSLSSTLTLSKADYEKHKVYACEVTHQGLSSPVTKSFNR
GEC
SEQ ID NO: 92:
SynOA DVD34-Amino Acid Tanezumab +
CRB0017 DVD Heavy Chain w LL and AA mutant
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPSVFPLAPEVQLVESGGG
LVQPGGSLRLSCAASGFTFSSYWMHWVRQAPGKGLEWVAYINPSTGYT
EYNQKFKRFTISRDDSKNTVYLQMNSLRAEDTAVYYCTSGGYDDLGYW
GQGTLVTVSSASTKGPASTKGPSVFPLAPSSKSTSGGTAALGCLVKDY
FPEPVTVSWNSGALTSGVHTFPAVLQSSGLYSLSSVVTVPSSSLGTQT
YICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKG
QPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYT
QKSLSLSPGK
SEQ ID NO: 93:
SynOA DVD34-Amino Acid Tanezumab +
CRB0017 DVD Light Chain w LL and AA mutant
DIQMTQSPSSLSASVGDRVTITCRASQSISNNLNWYQQKPGKAPKLLI
YYTSRFHSGVPSRFSGSGSGTDFTFTISSLQPEDIATYYCQQEHTLPY
TFGQGTKLEIKRTVAAPSVFIFPPDIQMTQSPSSLSASVGDRVTITCR
SSKSLLYKDGKTYLYWYQQKPGKAPKLLIYLMSTRASGVPSRFSGSGS
GTDFTLTISSLQPEDFATYYCQQLVUYPYTFGQGTKLEIKRTVAAPSV
FIFPPSDEQLKSGTASVVCLLNNFYPREAKVQWKVDNALQSGNSQESV
TEQDSKDSTYSLSSTLTLSKADYEKHKVYACEVTHQGLSSPVTKSFNR
GEC
SEQ ID NO: 94:
SynOA DVD35-Amino Acid Fasinumab +
CRB0017 DVD Heavy Chain w LL and AA mutant
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPSVFPLAPEVQLVESGGGLVQ
PGGSLRLSCAASGFTFSSYWMHWVRQAPGKGLEWVAYINPSTGYTEYN
QKFKRFTISRDDSKNTVYLQMNSLRAEDTAVYYCTSGGYDDLGYWGQG
TLVTVSSASTKGPASTKGPSVFPLAPSSKSTSGGTAALGCLVKDYFPE
PVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKS
LSL
SEQ ID NO: 95:
SynOA DVD35-Amino Acid Fasinumab +
CRB0017 DVD Light Chain w LL and AA mutant
DIQMTQSPSSLSASVGDRVTITCKASQSVGTTIVWTQQLPGLAPKLLI
YSASNRHTGVPSRFSGSGSGTDFTLTISSLQPEDFATYYCQQYTSYPF
TFGQGTKEIKRTVAAPSVFIFPPDIQMTQSPSSLSASVGDRVTITCRS
SKSLLYKDGKTYLYWYQQKPGKAPKLLIYLMSTRASGVPSRFSGSGSG
TDFTLTISSLQPEDFATYYCQQLVUYPYTFGQGTKLEIKRTVAAPSVF
IFPPSDEQLKSGTASVVCLLNNFYPREAKVQWKVDNALQSGNSQESVT
EQDSKDSTYSLSSTLTLSKADYEKHKVYACEVTHQGLSSPVTKSFNRG
EC
SEQ ID NO: 96:
SynOA DVD36-Amino Acid Fulranumab +
CRB0017 DVD Heavy Chain w LL and AA mutant
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPSVFPLAPEVQLVESG
GGLVQPGGSLRLSCAASGFTFSSYWMHWVRQAPGKGLEWVAYINPSTG
YTEYNQKFKRFTISRDDSKNTVYLQMNSLRAEDTAVYYCTSGGYDDLG
YWGQGTLVTVSSASTKGPASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNH
YTQKSLSL
SEQ ID NO: 97:
SynOA DVD36-Amino Acid Fulranumab +
CRB0017 DVD Light Chain w LL and AA mutant
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPSVFIFPPDIQMTQSPSSLSASVGDRVTITCR
SSKSLLYKDGKTYLYWYQQKPGKAPKLLIYLMSTRASGVPSRFSGSGS
GTDFTLTISSLQPEDFATYYCQQLVUYPYTFGQGTKLEIKRTVAAPSV
FIFPPSDEQLKSGTASVVCLLNNFYPREAKVQWKVDNALQSGNSQESV
TEQDSKDSTYSLSSTLTLSKADYEKHKVYACEVTHQGLSSPVTKSFNR
GEC
SEQ ID NO: 98:
SynOA DM9-Amino Acid Fasinumab Heavy Chain
with AA and Duobody (K409R) Mutants
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPSVFPLAPSSKSTSGGTAALG
CLVKDYFPEPVTVSWNSGALTSGVHTFPAVLQSSGLYSLSSVVTVPSS
SLGTQTYICNVNHKPSNTKVDKKVEPKSCDKTHTCPPCPAPEAAGGPS
VFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWES
NGQPENNYKTTPPVLDSDGSFFLYSRLTVDKSRWQQGNVFSCSVMHEA
LHNHYTQKSLSLSPGK
SEQ ID NO: 99:
SynOA DM9-Amino Acid NGF (Fasinumab) Light Chain
DIQMTQSPSSLSASAGDRVTITCRASQAIRNDLGWYQQKPGKAPKRLI
YAAFNLQSGVPSRFSGSGSGTEFTLTISSLQPEDLASYYCQQYNRYPW
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGE
C
SEQ ID NO: 100:
SynOA DM10-Amino Acid 7B4 Heavy Chain
with AA and Duobody (K409R) Mutants
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSRDELTKNQVSLSCLVKGFYPSDI
AVEWESNGQPENNYKTTPPVLDSDGSFFLYSRLTVDKSRWQQGNVFSC
SVMHEALHNHYTQKSLSLSPGK
SEQ ID NO: 101:
SynOA DM10-Amino Acid Fasinumab Heavy Chain
with AA and Duobody (F405L)
Mutants
QVQLVQSGAEVKKPGASVKVSCKVSGFTLTELSIHWVRQAPGKGLEWM
GGFDPEDGETIYAQKFQGRVTMTEDTSTDTAYMELTSLRSEDTAVYYC
STIGVVTNFDNWGQGTLVTVSSASTKGPSVFPLAPSSKSTSGGTAALG
CLVKDYFPEPVTVSWNSGALTSGVHTFPAVLQSSGLYSLSSVVTVPSS
SLGTQTYICNVNHKPSNTKVDKKVEPKSCDKTHTCPPCPAPEAAGGPS
VFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWES
NGQPENNYKTTPPVLDSDGSFKLYSKLTVDKSRWQQGNVFSCSVMHEA
LHNHYTQKSLSLSPGK
SEQ ID NO: 102 ADAMTS5 (12F4) HC with AA and
Duobody (K409R) Mutants
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLSCLVKGFYPSDIAVEWESNGQP
ENNYKTTPPVLDSDGSFFLYSRLTVDKSRWQQGNVFSCSVMHEALHNH
YTQKSLSLSPGK
SEQ ID NO: 103:
SynOA CM3-Amino Acid Fulranumab Heavy Chain
with AA and KIH mutations
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASVAAPSVFIFPPSDEQLKSG
TASVVCLLNNFYPREAKVQWKVDNALQSGNSQESVTEQDSKDSTYSLS
STLTLSKADYEKHKVYACEVTHQGLSSPVTKSFNRGECDKTHTCPPCP
APEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNK
ALPAPIEKTISKAKGQPREPQVYTLPPCRDELTKNQVSLWCLVKGFYP
SDIAVEWESNGQPENNYKTTPPVLDSDGSFFLYSKLTVDKSRWQQGNV
FSCSVMHEALHNHYTQKSLSLSPGK
SEQ ID NO: 104:
SynOA CM3-Amino Acid NGF (Fulranumab) Light
Chain
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKVTVLSSASTKGPSVFPLAPSSKSTSGGTAALGCLVKDYFPE
PVTVSWNSGALTSGVHTFPAVLQSSGLYSLSSVVTVPSSSLGTQTYIC
NVNHKPSNTKVDKKVEPKSC
SEQ ID NO: 105:
SynOA DVD5-Amino Acid 7B4 +
Fulranumab DVD Heavy Chain w SS and AA
Mutaton
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPEVQLVESGGGLVQP
GGSLRLSCAASGFTLRSYSMNWVRQAPGKGLEWVSYISRSSHTIFYAD
SVKGRFTISRDNAKNSLYLQMDSLRDEDTAMYYCARVYSSGWHVSDYF
DYWGQGILVTVSSASTKGPSVFPLAPSSKSTSGGTAALGCLVKDYFPE
PVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKS
LSLSPGK
SEQ ID NO: 106:
SynOA DVD5-Amino Acid 7B4 +
Fulranumab DVD Light Chain w SS and AA Mutaton
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPAIQLTQSPSSLSASVGDRVTITCRASQGISS
ALAWYQQKPGKAPKLLIYDASSLESGVPSRFSGSGSGTDFTLTISSLQ
PEDFATYYCQQFNSYPLTFGGGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 107:
SynOA DVD7-Amino Acid 12F4 + Fulranumab
DVD Heavy Chain w SS and AA Mutation
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPEVQLVESGGGLVQPGGSLRLSCAA
SGFTLRSYSMNWVRQAPGKGLEWVSYISRSSHTIFYADSVKGRFTISR
DNAKNSLYLQMDSLRDEDTAMYYCARVYSSGWHVSDYFDYWGQGILVT
VSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPS
RDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 108:
SynOA DVD7-Amino Acid 12F4 +
Fulranumab DVD Light Chain w SS and AA
Mutation
DIQMTQSPSSLSASVGDRVTITCKASQSVGTTIVWTQQLPGLAPKLLI
YSASNRHTGVPSRFSGSGSGTDFTLTISSLQPEDFATYYCQQYTSYPF
TFGQGTKEIKRTVAAPAIQLTQSPSSLSASVGDRVTITCRASQGISSA
LAWYQQKPGKAPKLLIYDASSLESGVPSRFSGSGSGTDFTLTISSLQP
EDFATYYCQQFNSYPLTFGGGTKLEIKRTVAAPSVFIFPPSDEQLKSG
TASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 109:
SynOA DVD6-Amino Acid Fulranumab +
7B4 DVD Heavy Chain w SS and AA Mutation
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPEVQLVESGGGLVQPG
GSLRLSCAASGFTFSDAWMDWVRQAPGKGLEWVAEIRNKANNHARHYA
ESVKGRFTISRDNAKNSLYLQMNSLRAEDTAVYYCARTYYYGSSYGYC
DVWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKS
LSLSPGK
SEQ ID NO: 110:
SynOA DVD6-Amino Acid Fulranumab +
7B4 DVD Light Chain w SS and AA Mutation
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPDIQMTQSPSSLSASVGDRVTITCRTSENIYS
YLAWYQQKPGKAPKLLIYNAKTLAEGVPSRFSGSGSGTDFTLTISSLQ
PEDFATYYCQHHYGTPWTFGQGTKLEIKRTVAAPSVFIFPPSDEQLKS
GTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 111:
SynOA DVD8-Amino Acid Fulranumab +
7B4 DVD Heavy Chain w SS and AA
Mutation
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPEVQLLESGGGLVQPG
GSLRLSCAASGFTFSDAWMDWVRQAPGKGLEWVAEIRHKANDHAIFYD
ESVKGRFTISRDSKNTVYLQMNSLRAEDTAVYYCTSPFAYWGQGTLVT
VSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPE
VTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPS
RDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 112:
SynOA DVD8-Amino Acid Fulranumab +
7B4 DVD Light Chain w SS and AA Mutation
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPDIQMTQSPSSLSASVGDRVTITCKASQSVGT
TIVWTQQLPGLAPKLLIYSASNRHTGVPSRFSGSGSGTDFTLTISSLQ
PEDFATYYCQQYTSYPFTFGQGTKEIKRTVAAPSVFIFPPSDEQLKSG
TASVVCLLNNFYPREAKVQWKVDNALQSGNSQESVTEQDSKDSTYSLS
STLTLSKADYEKHKVYACEVTHQGLSSPVTKSFNRGEC
Seq #113:
SynOA DVD13-Amino Acid 7B4 +
Fulranumab DVD Heavy Chain w LL and AA Mutation
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPEVQLVES
GGGLVQPGGSLRLSCAASGFTLRSYSMNWVRQAPGKGLEWVSYISRSS
HTIFYADSVKGRFTISRDNAKNSLYLQMDSLRDEDTAMYYCARVYSSG
WHVSDYFDYWGQGILVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK
SEQ ID NO: 114:
SynOA DVD13-Amino Acid 7B4 +
Fulranumab DVD Light Chain w LL and AA
Mutation
DIQMTQSPSSLSASVGDRVTITCRTSENIYSYLAWYQQKPGKAPKLLI
YNAKTLAEGVPSRFSGSGSGTDFTLTISSLQPEDFATYYCQHHYGTPW
TFGQGTKLEIKRTVAAPSVFIFPPAIQLTQSPSSLSASVGDRVTITCR
ASQGISSALAWYQQKPGKAPKLLIYDASSLESGVPSRFSGSGSGTDFT
LTISSLQPEDFATYYCQQFNSYPLTFGGGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 115:
SynOA DVD15-Amino Acid 12F4 +
Fulranumab DVD Heavy Chain w LL and AA
Mutation
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPEVQLVESGGGLVQPGGS
LRLSCAASGFTLRSYSMNWVRQAPGKGLEWVSYISRSSHTIFYADSVK
GRFTISRDNAKNSLYLQMDSLRDEDTAMYYCARVYSSGWHVSDYFDYW
GQGILVTVSSASTKGPSVFPLAPSSKSTSGGTAALGCLVKDYFPEPVT
VSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSL
SPGK
SEQ ID NO: 116:
SynOA DVD15-Amino Acid 12F4 +
Fulranumab DVD Light Chain w LL and AA
Mutation
DIQMTQSPSSLSASVGDRVTITCKASQSVGTTIVWTQQLPGLAPKLLI
YSASNRHTGVPSRFSGSGSGTDFTLTISSLQPEDFATYYCQQYTSYPF
TFGQGTKEIKRTVAAPSVFIFPPAIQLTQSPSSLSASVGDRVTITCRA
SQGISSALAWYQQKPGKAPKLLIYDASSLESGVPSRFSGSGSGTDFTL
TISSLQPEDFATYYCQQFNSYPLTFGGGTKLEIKRTVAAPSVFIFPPS
DEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID NO: 117:
SynOA DVD14-Amino Acid Fulranumab +
7B4 DVD Heavy Chain
w LL and AA
Mutation
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPSVFPLAPEVQLVESG
GGLVQPGGSLRLSCAASGFTFSDAWMDWVRQAPGKGLEWVAEIRNKAN
NHARHYAESVKGRFTISRDNAKNSLYLQMNSLRAEDTAVYYCARTYYY
GSSYGYCDVWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK
SEQ ID NO: 118:
SynOA DVD14-Amino Acid Fulranumab +
7B4 DVD Light Chain w LL and AA
Mutation
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPSVFIFPPDIQMTQSPSSLSASVGDRVTITCR
TSENIYSYLAWYQQKPGKAPKLLIYNAKTLAEGVPSRFSGSGSGTDFT
LTISSLQPEDFATYYCQHHYGTPWTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 119:
SynOA DVD16-Amino Acid Fulranumab +
12F4 DVD Heavy Chain
w LL and AA
Mutation
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPSVFPLAPEVQLLESG
GGLVQPGGSLRLSCAASGFTFSDAWMDWVRQAPGKGLEWVAEIRHKAN
DHAIFYDESVKGRFTISRDSKNTVYLQMNSLRAEDTAVYYCTSPFAYW
GQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVN
HKPSNTKVDKKVEPKSCDKTHTCPPCPAPEAAGGPSVFLFPPKPKDTL
MISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSL
SPGK
SEQ ID NO: 120:
SynOA DVD16-Amino Acid Fulranumab +
12F4 DVD Light Chain w LL and AA
Mutation
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPSVFIFPPDIQMTQSPSSLSASVGDRVTITCK
ASQSVGTTIVWTQQLPGLAPKLLIYSASNRHTGVPSRFSGSGSGTDFT
LTISSLQPEDFATYYCQQYTSYPFTFGQGTKEIKRTVAAPSVFIFPPS
DEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID NO: 121:
SynOA DM5-Amino Acid Fulranumab Heavy Chain
with AA and Duobody (K409R)
Mutants
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPSVFPLAPSSKSTSGG
TAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIA
VEWESNGQPENNYKTTPPVLDSDGSFFLYSRLTVDKSRWQQGNVFSCS
VMHEALHNHYTQKSLSLSPGK
SEQ ID NO: 122:
SynOA DM5-Amino Acid Fulranumab Light Chain
with AA and Duobody (K409R)
Mutants
AIQLTQSPSSLSASVGDRVTITCRASQGISSALAWYQQKPGKAPKLLI
YDASSLESGVPSRFSGSGSGTDFTLTISSLQPEDFATYYCQQFNSYPL
TFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 123:
SynOA DM6-Amino Acid Fulranumab Heavy Chain
with AA and Duobody (F405L)
Mutants
EVQLVESGGGLVQPGGSLRLSCAASGFTLRSYSMNWVRQAPGKGLEWV
SYISRSSHTIFYADSVKGRFTISRDNAKNSLYLQMDSLRDEDTAMYYC
ARVYSSGWHVSDYFDYWGQGILVTVSSASTKGPSVFPLAPSSKSTSGG
TAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIA
VEWESNGQPENNYKTTPPVLDSDGSFLLYSKLTVDKSRWQQGNVFSCS
VMHEALHNHYTQKSLSLSPGK
SEQ ID NO: 124:
SynOA DM2-Amino Acid Tanezumab Heavy Chain
w AA and Duobody (F405L)
Mutants
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFLLYSKLTVDKSRWQQGNVFSCSVM
HEALHNHYTQKSLSLSPGK
SEQ ID NO: 125:
SynOA DM4-Amino Acid Tanezumab Heavy Chain
w AA and Duobody (F405L)
Mutants
QVQLQESGPGLVKPSETLSLTCTVSGFSLIGYDLNWIRQPPGKGLEWI
GIIWGDGTTDYNSAVKSRVTISKDTSKNQFSLKLSSVTAADTAVYYCA
RGGYWYATSYYFDYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFLLYSKLTVDKSRWQQGNVFSCSVM
HEALHNHYTQKSLSLSPGK
SEQ ID NO: 126:
SynOA CM11-Amino Acid M6495 Heavy Chain
with AA and KIH Mutants
EVQLLESGGGLVQPGGSLRLSCAASGFTFSGRTVSSYAMGWVRQAPGK
GLEWVAGISRSAERTYRFTISRDSKNTVYLQMNSLRAEDTAVYYCTSD
LDPNRIFSREEYAYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVM
HEALHNHYTQKSLSLSPGK
SEQ ID NO: 127:
SynOA DM13-Amino Acid CRB0017 Heavy Chain
with AA and Duobody (F405L)
Mutants
EVQLLESGGGLVQPGGSLRLSCAASGFTFSSYWMHWVRQAPGKGLEWV
AYINPSTGYTEYNQKFKRFTISRDSKNTVYLQMNSLRAEDTAVYYCTS
GGYDDLGYWGQGTLVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSRDELTKNQVSLSCLVKGFYPSDIAVEWESNGQ
PENNYKTTPPVLDSDGSFLLYSKLTVDKSRWQQGNVFSCSVMHEALHN
HYTQKSLSLSPGK
SEQ ID NO: 128:
SynOA DM13-Amino Acid CRB0017 Light Chain
DIQMTQSPSSLSASVGDRVTITCRSSKSLLYKDGKTYLYWTQQLPGLA
PKLLIYLMSTRASGVPSRFSGSGSGTDFTLTISSLQPEDFATYYCQQL
VUYPYTFGQGTKLEIKRTVAAPSVFIFPPSDEQLKSGTASVVCLLNNF
YPREAKVQWKVDNALQSGNSQESVTEQDSKDSTYSLSSTLTLSKADYE
KHKVYACEVTHQGLSSPVTKSFNRGEC
SEQ ID NO: 129:
SynOA DM14-Amino Acid CRB0017 Heavy Chain
with AA and Duobody (K409R)
Mutants
EVQLLESGGGLVQPGGSLRLSCAASGFTFSSYWMHWVRQAPGKGLEWV
AYINPSTGYTEYNQKFKRFTISRDSKNTVYLQMNSLRAEDTAVYYCTS
GGYDDLGYWGQGTLVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSRDELTKNQVSLSCLVKGFYPSDIAVEWESNGQ
PENNYKTTPPVLDSDGSFFLYSRLTVDKSRWQQGNVFSCSVMHEALHN
HYTQKSLSLSPGK
SEQ ID NO: 130:
SynOA BF2-Amino Acid 7B4 Heavy Chain
AA mutant + Fulranumab scFv
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSRDELTKNQVSLTCLVKGFYPSDI
AVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGL
VQPGGSLRLSCAASGFTLRSYSMNWVRQAPGKGLEWVSYISRSSHTIF
YADSVKGRFTISRDNAKNSLYLQMDSLRDEDTAMYYCARVYSSGWHVS
DYFDYWGQGILVTVSSGGGGSGGGGSGGGGSAIQLTQSPSSLSASVGD
RVTITCRASQGISSALAWYQQKPGKAPKLLIYDASSLESGVPSRFSGS
GSGTDFTLTISSLQPEDFATYYCQQFNSYPLTFGGGTKLEIK
SEQ ID NO: 131:
SynOA BF3-Amino Acid 7B4 HC with AA mutant +
NGF (Fasinumab) scFv
EVQLVESGGGLVQPGGSLRLSCAASGFTFSDAWMDWVRQAPGKGLEWV
AEIRNKANNHARHYAESVKGRFTISRDNAKNSLYLQMNSLRAEDTAVY
YCARTYYYGSSYGYCDVWGQGTLVTVSSASTKGPSVFPLAPSSKSTSG
GTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALP
APIEKTISKAKGQPREPQVYTLPPSRDELTKNQVSLTCLVKGFYPSDI
AVEWESNGQPENNYKTTPPVLDSDGSFFLYSKLTVDKSRWQQGNVFSC
SVMHEALHNHYTQKSLSLSPGKGGGGSGGGGSGGGGSQVQLVQSGAEV
KKPGASVKVSCKVSGFTLTELSIHWVRQAPGKGLEWMGGFDPEDGETI
YAQKFQGRVTMTEDTSTDTAYMELTSLRSEDTAVYYCSTIGVVTNFDN
WGQGTLVTVSSGGGGSGGGGSGGGGSDIQMTQSPSSLSASAGDRVTIT
CRASQAIRNDLGWYQQKPGKAPKRLIYAAFNLQSGVPSRFSGSGSGTE
FTLTISSLQPEDLASYYCQQYNRYPWTFGQGTKLEIK
SEQ ID NO: 132:
SynOA BF5-Amino Acid 12F4 HC with AA mutant +
NGF (Fulranumab) scFv
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNH
YTQKSLSLSPGKGGGGSGGGGSGGGGSEVQLVESGGGLVQPGGSLRLS
CAASGFTLRSYSMNWVRQAPGKGLEWVSYISRSSHTIFYADSVKGRFT
ISRDNAKNSLYLQMDSLRDEDTAMYYCARVYSSGWHVSDYFDYWGQGI
LVTVSSGGGGSGGGGSGGGGSAIQLTQSPSSLSASVGDRVTITCRASQ
GISSALAWYQQKPGKAPKLLIYDASSLESGVPSRFSGSGSGTDFTLTI
SSLQPEDFATYYCQQFNSYPLTFGGGTKLEIK
SEQ ID NO: 133:
SynOA BF6-Amino Acid 12F4 HC with AA mutant +
Fasinumab scFv
EVQLLESGGGLVQPGGSLRLSCAASGFTFSDAWMDWVRQAPGKGLEWV
AEIRHKANDHAIFYDESVKGRFTISRDSKNTVYLQMNSLRAEDTAVYY
CTSPFAYWGQGTLVTVSSASTKGPSVFPLAPSSKSTSGGTAALGCLVK
DYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKA
KGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNH
YTQKSLSLSPGKGGGGSGGGGSGGGGSQVQLVQSGAEVKKPGASVKVS
CKVSGFTLTELSIHWVRQAPGKGLEWMGGFDPEDGETIYAQKFQGRVT
MTEDTSTDTAYMELTSLRSEDTAVYYCSTIGVVTNFDNWGQGTLVTVS
SGGGGSGGGGSGGGGSDIQMTQSPSSLSASAGDRVTITCRASQAIRND
LGWYQQKPGKAPKRLIYAAFNLQSGVPSRFSGSGSGTEFTLTISSLQP
EDLASYYCQQYNRYPWTFGQGTKLEIK
SEQ ID NO: 134:
SynOA BF7-Amino Acid M6495 HC with AA mutant +
Tanezumab scFv
EVQLLESGGGLVQPGGSLRLSCAASGFTFSGRTVSSYAMGWVRQAPGK
GLEWVAGISRSAERTYRFTISRDSKNTVYLQMNSLRAEDTAVYYCTSD
LDPNRIFSREEYAYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVM
HEALHNHYTQKSLSLSPGKGGGGSGGGGSGGGGSQVQLQESGPGLVKP
SETLSLTCTVSGFSLIGYDLNWIRQPPGKGLEWIGIIWGDGTTDYNSA
VKSRVTISKDTSKNQFSLKLSSVTAADTAVYYCARGGYWYATSYYFDY
WGQGTLVTVSSGGGGSGGGGSGGGGSDIQMTQSPSSLSASVGDRVTIT
CRASQSISNNLNWYQQKPGKAPKLLIYYTSRFHSGVPSRFSGSGSGTD
FTFTISSLQPEDIATYYCQQEHTLPYTFGQGTKLEIK
SEQ ID NO: 135:
SynOA BF8-Amino Acid M6495 HC with AA mutant +
Fulranumab scFv
EVQLLESGGGLVQPGGSLRLSCAASGFTFSGRTVSSYAMGWVRQAPGK
GLEWVAGISRSAERTYRFTISRDSKNTVYLQMNSLRAEDTAVYYCTSD
LDPNRIFSREEYAYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVM
HEALHNHYTQKSLSLSPGKGGGGSGGGGSGGGGSEVQLVESGGGLVQP
GGSLRLSCAASGFTLRSYSMNWVRQAPGKGLEWVSYISRSSHTIFYAD
SVKGRFTISRDNAKNSLYLQMDSLRDEDTAMYYCARVYSSGWHVSDYF
DYWGQGILVTVSSGGGGSGGGGSGGGGSAIQLTQSPSSLSASVGDRVT
ITCRASQGISSALAWYQQKPGKAPKLLIYDASSLESGVPSRFSGSGSG
TDFTLTISSLQPEDFATYYCQQFNSYPLTFGGGTKLEIK
SEQ ID NO: 136:
SynOA BF9-Amino Acid M6495 HC with AA mutant +
Fasinumab scFv
EVQLLESGGGLVQPGGSLRLSCAASGFTFSGRTVSSYAMGWVRQAPGK
GLEWVAGISRSAERTYRFTISRDSKNTVYLQMNSLRAEDTAVYYCTSD
LDPNRIFSREEYAYWGQGTLVTVSSASTKGPSVFPLAPSSKSTSGGTA
ALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVM
HEALHNHYTQKSLSLSPGKGGGGSGGGGSGGGGSQVQLVQSGAEVKKP
GASVKVSCKVSGFTLTELSIHWVRQAPGKGLEWMGGFDPEDGETIYAQ
KFQGRVTMTEDTSTDTAYMELTSLRSEDTAVYYCSTIGVVTNFDNWGQ
GTLVTVSSGGGGSGGGGSGGGGSDIQMTQSPSSLSASAGDRVTITCRA
SQAIRNDLGWYQQKPGKAPKRLIYAAFNLQSGVPSRFSGSGSGTEFTL
TISSLQPEDLASYYCQQYNRYPWTFGQGTKLEIK
SEQ ID NO: 137:
SynOA BF10-Amino Acid CRB0017 HC with
AA mutant + Tanezumab scFv
EVQLLESGGGLVQPGGSLRLSCAASGFTFSSYWMHWVRQAPGKGLEWV
AYINPSTGYTEYNQKFKRFTISRDSKNTVYLQMNSLRAEDTAVYYCTS
GGYDDLGYWGQGTLVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHN
HYTQKSLSLSPGKGGGGSGGGGSGGGGSQVQLQESGPGLVKPSETLSL
TCTVSGFSLIGYDLNWIRQPPGKGLEWIGIIWGDGTTDYNSAVKSRVT
ISKDTSKNQFSLKLSSVTAADTAVYYCARGGYWYATSYYFDYWGQGTL
VTVSSGGGGSGGGGSGGGGSDIQMTQSPSSLSASVGDRVTITCRASQS
ISNNLNWYQQKPGKAPKLLIYYTSRFHSGVPSRFSGSGSGTDFTFTIS
SLQPEDIATYYCQQEHTLPYTFGQGTKLEIK
SEQ ID NO: 138:
SynOA BF11-Amino Acid CRB0017 HC with AA mutant +
Fulranumab scFv
EVQLLESGGGLVQPGGSLRLSCAASGFTFSSYWMHWVRQAPGKGLEWV
AYINPSTGYTEYNQKFKRFTISRDSKNTVYLQMNSLRAEDTAVYYCTS
GGYDDLGYWGQGTLVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHN
HYTQKSLSLSPGKGGGGSGGGGSGGGGSEVQLVESGGGLVQPGGSLRL
SCAASGFTLRSYSMNWVRQAPGKGLEWVSYISRSSHTIFYADSVKGRF
TISRDNAKNSLYLQMDSLRDEDTAMYYCARVYSSGWHVSDYFDYWGQG
ILVTVSSGGGGSGGGGSGGGGSAIQLTQSPSSLSASVGDRVTITCRAS
QGISSALAWYQQKPGKAPKLLIYDASSLESGVPSRFSGSGSGTDFTLT
ISSLQPEDFATYYCQQFNSYPLTFGGGTKLEIK
SEQ ID NO: 139:
SynOA BF12-Amino Acid CRB0017 HC with AA mutant +
Fasinumab scFv
EVQLLESGGGLVQPGGSLRLSCAASGFTFSSYWMHWVRQAPGKGLEWV
AYINPSTGYTEYNQKFKRFTISRDSKNTVYLQMNSLRAEDTAVYYCTS
GGYDDLGYWGQGTLVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHN
HYTQKSLSLSPGKGGGGSGGGGSGGGGSQVQLVQSGAEVKKPGASVKV
SCKVSGFTLTELSIHWVRQAPGKGLEWMGGFDPEDGETIYAQKFQGRV
TMTEDTSTDTAYMELTSLRSEDTAVYYCSTIGVVTNFDNWGQGTLVTV
SSGGGGSGGGGSGGGGSDIQMTQSPSSLSASAGDRVTITCRASQAIRN
DLGWYQQKPGKAPKRLIYAAFNLQSGVPSRFSGSGSGTEFTLTISSLQ
PEDLASYYCQQYNRYPWTFGQGTKLEIK
Target Sequences and Species Homology
(Mature Amino Acid Sequence)
SEQ ID NO: 140:
Human ADAMTS5
MLLGWASLLLCAFRLPLAAVGPAATPAQDKAGQPPTAAAAAQPRRRQG
EEVQERAEPPGHPHPLAQRRRSKGLVQNIDQLYSGGGKVGYLVYAGGR
RFLLDLERDGSVGIAGFVPAGGGTSAPWRHRSHCFYRGTVDGSPRSLA
VFDLCGGLDGFFAVKHARYTLKPLLRGPWAEEEKGRVYGDGSARILHV
YTREGFSFEALPPRASCETPASTPEAHEHAPAHSNPSGRAALASQLLD
QSALSPAGGSGPQTWWRRRRRSISRARQVELLLVADASMARLYGRGLQ
HYLLTLASIANRLYSHASIENHIRLAVVKVVVLGDKDKSLEVSKNAAT
TLKNFCKWQHQHNQLGDDHEEHYDAAILFTREDLCGHHSCDTLGMADV
GTICSPERSCAVIEDDGLHAAFTVAHEIGHLLGLSHDDSKFCEETFGS
TEDKRLMSSILTSIDASKPWSKCTSATITEFLDDGHGNCLLDLPRKQI
LGPEELPGQTYDATQQCNLTFGPEYSVCPGMDVCARLWCAVVRQGQMV
CLTKKLPAVEGTPCGKGRICLQGKCVDKTKKKYYSTSSHGNWGSWGSW
GQCSRSCGGGVQFAYRHCNNPAPRNNGRYCTGKRAIYRSCSLMPCPPN
GKSFRHEQCEAKNGYQSDAKGVKTFVEWVPKYAGVLPADVCKLTCRAK
GTGYYVVFSPKVTDGTECRLYSNSVCVRGKCVRTGCDGIIGSKLQYDK
CGVCGGDNSSCTKIVGTFNKKSKGYTDVVRIPEGATHIKVRQFKAKDQ
TRFTAYLALKKKNGEYLINGKYMISTSETIIDINGTVMNYSGWSHRDD
FLHGMGYSATKEILIVQILATDPTKPLDVRYSFEVPKKSTPKVNSVTS
HGSNKVGSHTSQPQWVTGPWLACSRTCDTGWHTRTVQCQDGNRKLAKG
CPLSQRPSAFKQCLLKKC
SEQ ID NO: 141:
Cynomolgus ADAMTS5
MLLGWASLLLCAFRLPLAAAGPAAAPAQDKAGQPATAAAAAQPSRRQG
EEVQERTEPPGHPHPLAQRRSSKGLVQNIDQLYSGGGKVGYLVYAGGR
RFLLDLERDGSVGTAGFVPTGGGTSAPWRHRSHCFYRGTVDGSPRSLA
VFDLCGGLDGFFAVKHARYTLKPLLRGPWAEEETGRVYGDGSARILHV
YTREGFSFEALQPRASCETPASTPEPHERPPAHSNPGGRAALASQLLD
QSAVSPAGGPGPQTWWRRRRRSISRARQVELLLVADASMARLYGRGLQ
HYLLTLASIANRLYSHASIENHIRLAVVKVVVLGDKDKSLEVSKNAAT
TLKNFCKWQHQHNQLGDDHEEHYDAAILFTREDLCGHHSCDTLGMADV
GTICSPERSCAVIEDDGLHAAFTVAHEIGHLLGLSHDDSKFCEETFGS
TEDKRLMSSILTSIDASKPWSKCTSATITEFLDDGHGNCLLDQPRKQI
LGPEELPGQTYDATQQCNLTFGPEYSVCPGMDVCARLWCAVVRQGQMV
CLTKKLPAVEGTPCGKGRICLQGKCVDKTKKKYYSTSSHGNWGSWGSW
GQCSRSCGGGVQFAYRHCNNPAPRNNGRYCTGKRAIYRSCGLMPCPPN
GKSFRHEQCEAKNGYQSDAKGVKTFVEWVPKYAGVLPADVCKLTCRAK
GTGYYVVFSPKVTDGTECRPYSNSVCVRGKCVRTGCDSIIGSKLQYDK
CGVCGGDNSSCTKIVGTFNKKSKGYTDVVRIPEGATHIKVRQFKAKDQ
TRFTAYLALKKKNGEYLINGKYMISTSETIIDINGTVMNYSGWSHRDD
FLHGMGYSATKEILIVQILATDPTKPLDVRYSFFVPKKSTPKVNSVTS
HGSNKVGSHTSQLQWVTGPWLACSRTCDTGWHTRTVQCQDGNRKLAKG
CPLSQRPSAFKQCLLKKC
SEQ ID NO: 142:
Feline ADAMTS5
MLLGWASLLLCALRLPPVAAGPAAAPAQDTAGQPRAAAAAAQPRGRQG
EEAQERAEPPGHPHPLAPQRRSGGLVHNIDQIYAGGGKVGYLVYAGGR
RFLLDLERDGSLGAAGFAPAGSGPGASRRHRDHCFHRGTVDGSPRSLA
VFDLCGGLDGFFAVKHARYTLKPLLRGPRAEAEAGRVYGDGSSRVLHV
YTREGFSFEAVPPRASCETPASPPGPRERPPAHGGPGPRWELAPPFPD
QTVPSSEGTQGPQTWWRRRRRSISRARQVELLLVADASMARMYGRGLQ
HYLLTLASIANRLYSHASIENHIRLAVVKVVVLGDKDKSLEVSKNAAT
TLKNFCKWQHQHNQLEDDHEEHHDAAILFTREDLCGHHSCDTLGMADV
GTICSPERSCAVIEDDGLHAAFTVAHEIGHLLGLSHDDSKFCEENFGS
TEDKRLMSSILTSIDASKPWSKCTSATITEFLDDGHGNCLLDVPRQQI
SGPEELPGQTYDATQQCNLTFGPEYSVCPGMDVCTRLWCAVVRQGQMV
CLTKKLPAVEGTPCGKGRICLQGKCVDKTKKKYYSTSSHGNWGSWGPW
GQCSRSCGGGVQFAYRHCNNPAPRNSGRYCTGKRAIYRSCSVTPCPPN
GKSFRHEQCEAKNGYQSDAKGVKTFVEWVPKYAGVLLGDVCKLTCRAK
GTGYYVVFSPKVTDGTECRPYSNSVCVRGKCVRTGCDGIIGSRLQYDK
CAVCGGDNSSCTKVVGTFNKKSKGYTDVVRIPEGATHIKVRQFKAKDQ
TRFTAYLALKKKNGEYLINGKYMISTSETIIDINGTVMNYSGWSHRDD
FLHGMGYSATKEILIVQILATDPTKALDVRYSFFVPKKSTQKVNSVSS
HGSNKVGSHTPQLQWVTGPWLACSRTCDTGWHTRTVQCQDANRKLAKG
CLLSQRPSAFKQCLLKKC
SEQ ID NO: 143:
Canine ADAMTS5
MLLGWASLLLGALRLPPVAAGPAAAPAQDKAGQPWAAAAAAQPRRRQG
EEAREPAEPPGHPHPLAPQRRSSGLVQNVDQIYAGGGKVGYLVYAGGR
RFLLDLERDGSVGAAGSAPAGRGPGAPRRHRDHCFYRGTVDGSPRSLA
VLDLCGGLDGFFAVRHARYTLKPLLRGPWAGAGAGAEAERVYGDGSPR
ILHVYTREGFSFEALPPRTSCETPASPPGPRERPPAHSSPDPRWSPAP
PFPAPPAASPDGGPGPQTWWRRRRRSISRARQVELLLVADASMARMYG
RGLQHYLLTLASIANRLYSHASIENHIRLAVVKVVVLGDKDKSLEVSK
NAATTLKNFCKWQHQHNQLGDDHEEHYDAAILFTREDLCGHHSCDTLG
MADVGTICSPERSCAVIEDDGLHAAFTVAHEIGHLLGLSHDDSKFCEE
NFGSTEDKRLMSSILTSIDASKPWSKCTSATITEFLDDGHGNCLLDLP
RKQILGPEELPGQTYDATQQCNLTFGPEYSVCPGMDVCARLWCAVVRQ
GQMVCLTKKLPAVEGTPCGKGRICLQGKCVDKTKKKYYSTSSHGNWGS
WGSWGQCSRSCGGGVQFAYRHCNNPAPRNNGRYCTGKRAIYRSCNVTP
CPPNGKSFRHEQCEAKNGYQSDAKGVKTFVEWVPKYAGVLLGDVCKLT
CRAKGTGYYVVFSPKVTDGTECRPYSNSVCVRGKCVRTGCDGIIGSKL
QYDKCAVCGGDNSSCTKVVGTFNKKSKGYTDVVRIPEGATHIKVRQFK
AKDQTRFTAYLALKKKNGEYLINGKYMISTSETIIDINGTVMNYSGWS
HRDDFLHGMGYSATKEILIVQILATDPTKALDVRYSFFVPKKSTQKVN
SVTSHASNKVGSHTPQLQWVTGPWLACSRTCDTGWHTRTVQCQDANRK
LAKGCLLSQRPSAFKQCLLKKC
SEQ ID NO: 144:
Horse ADAMTS5
MLLGWASLLLCALRLPLVAAGPAAAPAQDKTGQPRAAAAAAQPRRRQG
EEAQERAEPPGHPHPLAPQRRSSGLVQNIDQIYSGGGKVGYLVYAGGR
RFLLDLERDGSVGAAGFVPVGGGPSATRRHRGHCFYRGTVDGSPRSLA
VFDLCGGLDGFFAVKHARYTLKPLLRGPWAETETGRVYGDGSARILHV
YTREGFSFEALPPRTSCETPASPPGSRERPPAHSSPEPRWALAPQFPD
QSATSSDGGQGSQTWRRRRRSISRARQVELLLVADASMARMYGRGLQH
YLLTLASIANRLYSHASIENHIRLAVVKMVVLGDKDKSLEVSKNAATT
LKNFCKWQHQHNQLGDDHEEHYDAAILFTREDLCGHHSCDTLGMADVG
TICSPERSCAVIEDDGLHAAFTVAHEIGHLLGLSHDDSKFCEENFGST
EEKRLMSSILTSIDASKPWSKCTSATITEFLDDGHGNCLLDLPRKQIL
GPEELPGQTYDATQQCNLTFGPEYSVCPGMDVCARLWCAVVRQGQMVC
LTKKLPAVEGTPCGKGRICLQGKCVDKTKKKYYSTSSHGNWGSWGSWG
QCSRSCGGGVQFAYRHCNNPAPRNNGRYCTGKRAIYRSCSVTPCPANG
KSFRHEQCEAKNGYQSDAKGVKTFVEWVPKYAGVLPGDVCKLTCRAKG
TGYYVVFSPKVTDGTECRPYSNSVCVRGKCVRTGCDGIIGSKLQYDKC
GVCGGDNSSCTKVVGTFNKKSKGYTDVVRIPEGATHIKVRQFKTKDQT
RFTAYLALKKKNGEYLINGKYMISTSETIIDINGTVMNYSGWSHRDDF
LHGMGYSATKEILIVQILATDPTKALDVRYSFFVPKKPTQKGNSVTSH
GSNKVGSTTPQLQWVTGPWLACSRTCDTGWHTRTVQCQDGNRKLAKGC
LLSQRPSAFKQCLLKKC
SEQ ID NO: 145:
Mouse ADAMTS5
MRLEWAPLLLLLLLLSASCLSLAADSPAAAPAQDKTRQPQAAAAAAEP
DQPQGEETRERGHLQPLAGQRRSGGLVQNIDQLYSGGGKVGYLVYAGG
RRFLLDLERDDTVGAAGSIVTAGGGLSASSGHRGHCFYRGTVDGSPRS
LAVFDLCGGLDGFFAVKHARYTLKPLLRGSWAEYERIYGDGSSRILHV
YNREGFSFEALPPRASCETPASPSGPQESPSVHSRSRRRSALAPQLLD
HSAFSPSGNAGPQTWWRRRRRSISRARQVELLLVADSSMARMYGRGLQ
HYLLTLASIANRLYSHASIENHIRLAVVKVVVLTDKDTSLEVSKNAAT
TLKNFCKWQHQHNQLGDDHEEHYDAAILFTREDLCGHHSCDTLGMADV
GTICSPERSCAVIEDDGLHAAFTVAHEIGHLLGLSHDDSKFCEENFGT
TEDKRLMSSILTSIDASKPWSKCTSATITEFLDDGHGNCLLDLPRKQI
LGPEELPGQTYDATQQCNLTFGPEYSVCPGMDVCARLWCAVVRQGQMV
CLTKKLPAVEGTPCGKGRVCLQGKCVDKTKKKYYSTSSHGNWGSWGPW
GQCSRSCGGGVQFAYRHCNNPAPRNSGRYCTGKRAIYRSCSVTPCPPN
GKSFRHEQCEAKNGYQSDAKGVKTFVEWVPKYAGVLPADVCKLTCRAK
GTGYYVVFSPKVTDGTECRPYSNSVCVRGRCVRTGCDGIIGSKLQYDK
CGVCGGDNSSCTKIIGTFNKKSKGYTDVVRIPEGATHIKVRQFKAKDQ
TRFTAYLALKKKTGEYLINGKYMISTSETIIDINGTVMNYSGWSHRDD
FLHGMGYSATKEILIVQILATDPTKALDVRYSFFVPKKTTQKVNSVIS
HGSNKVGPHSTQLQWVTGPWLACSRTCDTGWHTRTVQCQDGNRKLAKG
CLLSQRPSAFKQCLLKKC
SEQ ID NO: 146:
Rat ADAMTS5
MRLEWASLLLLLLLLCASCLALAADNPAAAPAQDKTRQPRAAAAAAQP
DQRQWEETQERGHPQPLARQRRSSGLVQNIDQLYSGGGKVGYLVYAGG
RRFLLDLERDDTVGAAGGIVTAGGLSASSGHRGHCFYRGTVDGSPRSL
AVFDLCGGLDGFFAVKHARYTLKPLLRGSWAESERVYGDGSSRILHVY
TREGFSFEALPPRTSCETPASPSGAQESPSVHSSSRRRTELAPQLLDH
SAFSPAGNAGPQTWWRRRRRSISRARQVELLLVADSSMAKMYGRGLQH
YLLTLASIANRLYSHASIENHIRLAVVKVVVLTDKSLEVSKNAATTLK
NFCKWQHQHNQLGDDHEEHYDAAILFTREDLCGHHSCDTLGMADVGTI
CSPERSCAVIEDDGLHAAFTVAHEIGHLLGLSHDDSKFCEENFGSTED
KRLMSSILTSIDASKPWSKCTSATITEFLDDGHGNCLLDVPRKQILGP
EELPGQTYDATQQCNLTFGPEYSVCPGMDVCARLWCAVVRQGQMVCLT
KKLPAVEGTPCGKGRICLQGKCVDKTKKKYYSTSSHGNWGSWGPWGQC
SRSCGGGVQFAYRHCNNPAPRNSGRYCTGKRAIYRSCSVIPCPPNGKS
FRHEQCEAKNGYQSDAKGVKTFVEWVPKYAGVLPADVCKLTCRAKGTG
YYVVFSPKVTDGTECRPYSNSVCVRGRCVRTGCDGIIGSKLQYDKCGV
CGGDNSSCTKIIGTFNKKSKGYTDVVRIPEGATHIKVRQFKAKDQTRF
TAYLALKKKTGEYLINGKYMISTSETIIDINGTVMNYSGWSHRDDFLH
GMGYSATKEILIVQILATDPTKALDVRYSFFVPKKTTQKVNSVISHSS
NKVGLHSPQLQWVTGPWLACSRTCDTGWHTRTVQCQDGNRKLAKGCIL
SQRPSAFKQCLLKKC

ADAMTS5 Protein Sequence Homology (% Identity)

| | Human | Cynomolgus | Dog | Cat | Horse | Mouse | Rat |
|---|---|---|---|---|---|---|---|
| Human | | 97.74 | 93.1 | 91.40 | 93.66 | 90.8 | 91.6 |
| Cynomolgus | 97.74 | | 93.5 | 91.51 | 94.09 | 90.5 | 91.5 |
| Dog | 93.1 | 93.5 | | 94.33 | 94.54 | 92.0 | 92.3 |
| Cat | 91.40 | 91.51 | 94.33 | | 94.09 | 89.93 | 90.59 |
| Horse | 93.66 | 94.09 | 94.54 | 94.09 | | 91.26 | 91.69 |
| Mouse | 90.8 | 90.5 | 92.0 | 89.93 | 91.26 | | 96.7 |
| Rat | 91.6 | 91.5 | 92.3 | 90.59 | 91.69 | 96.7 | |

SEQ ID NO: 147: Human Beta-NGF
MSMLFYTLITAFLIGIQAEPHSESNVPAGHTIPQAHWTKLQHSLD
TALRRARSAPAAAIAARVAGQTRNITVDPRLFKKRRLRSPRVLFS
TQPPREAADTQDLDFEVGGAAPFNRTHRSKRSSSHPIFHRGEFSV
CDSVSVWVGDKTTATDIKGKEVMVLGEVNINNSVFKQYFFETKCR
DPNPVDSGCRGIDSKHWNSYCTTTHTFVKALTMDGKQAAWRFIRI
DTACVCVLSRKAVRRA
SEQ ID NO: 148: Cynomolgus Beta-NGF
MSMLFYTLITAFLIGIQAEPHSESNVPAGHTIPQAHWTKLQHSLD
TALRRVRSAPAVAIAARVAGQTRNITVDPRLFKKRRLRSPRVLFS
TQPPPEAADTQDLDFEVGGAAPFNRTHRSKRSSSHPIFHRGEFSV
CDSVSVWVGDKTTATDIKGKEVMVLGEVNINNSVFKQYFFETKCR
DPNPVDSGCRGIDSKHWNSYCTTTHTFVKALTMDGKQAAWRFIRI
DTACVCVLSRKAVRRA
SEQ ID NO: 149: Canine Beta-NGF
MSMLFYTLITALLIGIRAEPHPESHVPAGHAIPHAHWTKLQHSLD
TALRRARSAPAGAIAARVTGQTRNITVDPKLFKKRRLRSPRVLFS
THPPPVAADAQDLDLEAGSTASVNRTHRSKRSSSHPVFHRGEFSV
CDSVSVWVGDKTTATDIKGKEVMVLGEVNINNSVFKQYFFETKCR
DPTPVDSGCRGIDSKHWNSYCTTTHTFVKALTMDGKQAAWRFIRI
DTACVCVLSRKAGRRA
SEQ ID NO: 150: Feline Beta-NGF
MSMLSYTLITALLIGIQAEPHPESNVPAGHTIPQAHWTKLQHSLD
TALRRARSTPAGAIAARVAGQTRNITVDPKLFKKRRLRSPRVLFS
THPPPVAADTQGLDLEAGGAASFNRTHRSKRSSSHPVFHRGEFSV
CDSVSVWVGDKTTATDIKGKEVMVLGEVNINNSVFKQYFFETKCR
DPTPVDSGCRGIDSKHWNSYCTTTHTFVKALTMDGKQAAWRFIRI
DTACVCVLSRKAGRRA
SEQ ID NO: 151: Horse Beta-NGF
MSMLFYTLITALLIGTQAEPHTESNVPAGHAIPQAHWTKLQHSLD
TALRRARSAPARAIAARVAGQTRNITVDPKLFKKRRLRSPRVLFS
TQPPPVAADTQDLDFEAGGAASFNRTHRSKRSSSHPVFHRGEFSV
CDSVSVWVGDKTTATDIKGKEVMVLGEVNINNSVFKQYFFETKCR
DPNPVDSGCRGIDSKHWNSYCTTTHTFVKALTMDGKQAAWRFIRI
DTACVCVLSRKTGRKA
SEQ ID NO: 152: Mouse Beta-NGF
MSMLFYTLITAFLIGVQAEPYTDSNVPEGDSVPEAHWTKLQHSLD
TALRRARSAPTAPIAARVTGQTRNITVDPRLFKKRRLHSPRVLFS
TQPPPTSSDTLDLDFQAHGTIPFNRTHRSKRSSTHPVFHMGEFSV
CDSVSVWVGDKTTATDIKGKEVTVLAEVNINNSVFRQYFFETKCR
ASNPVESGCRGIDSKHWNSYCTTTHTFVKALTTDEKQAAWRFIRI
DTACVCVLSRKATRRG
SEQ ID NO: 153: Rat Beta-NGF
MSMLFYTLITAFLIGVQAEPYTDSNVPEGDSVPEAHWTKLQHSLD
TALRRARSAPAEPIAARVTGQTRNITVDPKLFKKRRLRSPRVLFS
TQPPPTSSDTLDLDFQAHGTISFNRTHRSKRSSTHPVFHMGEFSV
CDSVSVWVGDKTTATDIKGKEVTVLGEVNINNSVFKQYFFETKCR
APNPVESGCRGIDSKHWNSYCTTTHTFVKALTTDDKQAAWRFIRI
DTACVCVLSRKAARRG

BETA-NGF Protein Sequence Homology (% Identity)

| | Human | Cynomolgus | Dog | Cat | Horse | Mouse | Rat |
|---|---|---|---|---|---|---|---|
| Human | | 98.8 | 90.9 | 93.36 | 94.19 | 85.1 | 85.9 |
| Cynomolgus | 98.8 | | 90.9 | 93.36 | 94.19 | 83.5 | 86.2 |
| Dog | 90.9 | 90.9 | | 95.02 | 93.36 | 82.2 | 85.1 |
| Cat | 93.36 | 93.36 | 95.02 | | 95.02 | 82.16 | 85.06 |
| Horse | 94.19 | 94.19 | 93.36 | 95.02 | | 84.23 | 87.14 |
| Mouse | 85.1 | 83.5 | 82.2 | 82.16 | 84.23 | | 95.9 |
| Rat | 85.9 | 86.2 | 85.1 | 85.06 | 87.14 | 95.9 | |

1. A composition comprising:
binding protein with two or more antigen binding components,
wherein at least one antigen binding component is capable of binding to human ADAMTS5,
and wherein at least one antigen binding component is capable of binding to nerve growth factor (NGF).

2. The composition of claim 1, wherein said antigen binding component is an antibody or fragment thereof.

3. The composition of claim 1, wherein said antibody is a monoclonal antibody or fragment thereof.

4. The composition of claim 3, wherein said monoclonal antibody or fragment thereof is mouse, feline, canine, equine, chimeric, humanized, or fully human.

5. The composition of claim 1, wherein the antigen binding component capable of binding to NGF comprises a monoclonal antibody selected from the group including, but not limited to, tanezumab, fasinumab, fulranumab, TrkA Fc and/or p75NTR Fc.

6. The composition of claim 1, wherein antigen binding component capable of binding to NGF consists of tanezumab.

7. The composition of claim 1, wherein one antigen binding component selectively binds with high affinity to one or more ADAMTS5 molecules and wherein one antigen binding protein selectively binds with high affinity to one or more NGF molecules.

8. The composition of claim 1, further comprising pharmaceutically acceptable excipients.

9. The composition of claim 1, wherein the binding protein with two or more antigen binding components comprises a bispecific antibody, and wherein the bispecific antibody is represented by the constructs of FIG. 6.

10. A method for treating chronic pain in a subject, comprising administering to the subject at least one dose of a composition comprising binding protein with two or more antigen binding components,
wherein at least one antigen binding component is capable of binding to human ADAMTS5,
and wherein at least one antigen binding component is capable of binding to nerve growth factor (NGF).

11. The method of claim 10, wherein chronic pain is associated with at least one disease selected from the group consisting of: osteoarthritis, cancer, pain, neuropathic pain, postoperative pain, sports injuries, erosive arthritis, rheumatoid arthritis, psoriatic arthritis, Lyme arthritis, juvenile arthritis, ankylosing spondylosis, neuralgia, neuropathies, algesia, nerve injury, ischaemia, neurodegeneration, inflammatory diseases, cartilage degeneration, diseases affecting the larynx, trachea, auditory canal, intervertebral discs, ligaments, tendons, joint capsules or bone development, intervertebral disc degeneration, osteopenia, or periodontal diseases, acute joint injury, and/or a disease related to joint destruction.

12. The method of claim 11, wherein the chronic pain is associated with osteoarthritis.

13. The method of claim 11, wherein the chronic pain is associated with cartilage degeneration and wherein administering at least one dose of said composition reduces cartilage degeneration in said subject.

14. The method of claim 13, wherein the binding protein with two or more antigen binding components comprises a bispecific antibody, and wherein the bispecific antibody is represented by the constructs of FIG. 6.

15. The method of claim 13, wherein the composition is administered intraarticularly, intravenously, intramuscularly, subcutaneously, orally, intranasally, and/or via respiratory inhaler.

16. The method of claim 15, wherein the composition is injected at the site of chronic pain.

17. The method of claim 16, wherein the site of chronic pain is the neck, shoulder, elbow, wrist, fingers, hips, knees, spine or ankles.

18. The method of claim 17, wherein the administration of the composition enables the alleviation of pain and also the reduction of cartilage degeneration.

19. The method of claim 18, wherein the bispecific antibody localizes to the site of chronic pain and has sustained effect over an extended period of time.

20. The method of claim 19, wherein the bispecific antibody is administered twice daily, daily, every other day, every third day, three times per week, twice per week, weekly, biweekly, monthly, bimonthly, quarterly, semi-annually, or annually.


SEQUENCE LISTING (159 sequences)

SEQ ID NO: 1 - protein, 454 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 444 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
DIQMTQSPSSLSASVGDRVTITCKASQSVGTTIVWTQQLPGKAPKLLIYSASNRHTGVPSRFSGSGSGTDFTLTISSLQPEDFATYYCQQYTSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 446 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPEPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 214 aa, synthetic construct
  VARIANT 190: note = X can be any amino acid
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHXVYACEVTHQGLSSPVTKSFNRGEC

SEQ ID NO: 7 - protein, 449 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 214 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 445 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, synthetic construct
GRTVSSYAMG

SEQ ID NO: 12 - protein, 5 aa, synthetic construct
SYWMH

SEQ ID NO: 13 - protein, 16 aa, synthetic construct
RSSKSLLYKDGKTYLY

SEQ ID NO: 14 - protein, 460 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNDIEGRMDPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 614 aa, synthetic construct
AAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEIEGRIDPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 454 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - DNA, 1362 bp, synthetic construct
  source 1..1362: mol_type = other DNA
gaagtccaacttgtggaaagtggtgggggacttgtgcaacctggcggatcactccgtctcagttgtgccgctagtggttttaccttttccgatgcttggatggattgggttagacaagctcctgggaaaggattggagtgggttgcagaaatccgcaacaaggcaaataaccacgccagacactacgctgagagtgtcaaaggtcgctttactataagtagagataacgccaaaaatagtctttacttgcaaatgaactccctccgggccgaggacactgctgtttactactgcgcaaggacatactattacggtagctcatacggatattgtgatgtttggggacaaggtacactcgttaccgtcagtagcgctagcactaaggggccatcagtattccccctggctcccagctccaaatctacctcaggaggaacagcagctctgggttgccttgtaaaagactactttcctgaacctgttactgttagctggaatagtggagctcttacaagcggcgtacatactttccctgccgtgttgcagtctagtggtctttactctctttcttctgtagtaactgtacctagtagctccttgggtacacaaacctacatatgtaacgtcaatcataagcctagcaataccaaagtagataaaaaagttgagcctaaaagttgtgacaaaacccacacttgccctccctgccctgcacccgaggccgcagggggaccctcagtcttcctgtttcctccaaaacctaaagacactttgatgatatccaggacccccgaggtaacctgtgtggttgtagatgttagccatgaagatcccgaggtaaagttcaactggtatgttgatggggtagaggtgcataacgctaaaactaaacctcgggaggaacagtacaattcaacttacagggttgtaagcgtactcacagtcctgcaccaagactggctgaatggcaaggagtataaatgtaaagtatctaacaaggcactgcccgctcctattgaaaagacaatatcaaaagctaaggggcagccacgagaaccccaggtgtgcaccctccccccatccagagatgaacttaccaaaaatcaagtgagcttgtcctgcgctgtcaaaggattctatccttcagatatagccgtggagtgggaaagtaacggccagcctgaaaacaattataagacaacaccacccgtgctggacagcgatggtagtttttttctggtcagcaagttgactgtagataagtctcgatggcagcagggcaatgttttcagttgttctgtaatgcacgaagctcttcataatcactatactcagaagtctctttcacttagccccggcaaa

SEQ ID NO: 18 - protein, 214 aa, synthetic construct; identical to SEQ ID NO: 2

SEQ ID NO: 19 - DNA, 642 bp, synthetic construct
  source 1..642: mol_type = other DNA
gacatacaaatgactcaatctccatcttccttgtctgcttcagtaggagaccgagttacaattacttgccgcacttccgagaacatttactcatatctggcttggtatcagcaaaagccaggtaaggccccaaaactccttatctataatgctaagactcttgctgagggagtacccagtaggttttccggttccggttcaggcacagatttcacactcactatttcttcactccagcctgaggacttcgctacttattattgccaacatcactacgggacaccctggactttcgggcaagggactaaactggaaataaaacgtaccgtggctgccccaagtgtcttcatatttcccccatctgatgagcaactcaaatcaggtactgcttctgttgtttgccttctcaacaatttttatccacgtgaggcaaaagtccagtggaaggtggacaacgccctgcaatcaggtaacagtcaggagtcagtgacagaacaagatagcaaagacagtacttattccctttccagcaccctgaccctctctaaagctgactatgagaaacataaggtctacgcctgtgaagtaacacatcaaggtctttcatctccagtcaccaagtctttcaacaggggggagtgt

SEQ ID NO: 20 - protein, 455 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1365 bp, synthetic construct
  source 1..1365: mol_type = other DNA
caggtgcagctccaagaatcaggcccaggcttggtgaaaccctcagaaactctcagcctcacatgcactgtgtctggctttagcctgattggttatgacctcaactggattcgtcaaccccccggcaagggtctggaatggatcggaatcatctggggagatggaactacagactacaatagtgccgtaaagtcccgagtgacaataagtaaagacacatccaagaaccagttttcactcaaactttcctcagtcaccgctgctgacacagcagtctattattgcgcaagaggtggatactggtatgctacaagctactacttcgattactggggacagggtacccttgtgaccgtatcctccgcatccgtggcagcccctagtgtattcatcttccctccttcagatgagcaactcaagtcaggaactgcctcagtcgtgtgtttgttgaataacttctacccacgtgaagccaaagtccaatggaaggtcgataatgccttgcaatccgggaacagccaagagtcagtgaccgagcaggacagtaaagacagtacttactctttgtcatctacccttaccctttccaaggctgactacgagaaacataaggtgtacgcttgcgaagtaactcaccagggactcagtagcccagttaccaaatcattcaacagaggagaatgtgataagactcatacctgccccccttgtcctgctcccgaggctgctgggggcccatctgtctttctgtttcccccaaagcctaaggatactctgatgatttctcgaactcccgaggtcacttgcgtagtagtagacgtcagtcacgaagacccagaagtcaagttcaattggtatgtagatggggtagaagtgcataatgctaagactaaacctcgagaggagcagtacaactcaacttacagggtcgttagcgtattgaccgtcctccatcaagattggctcaacggaaaagaatacaaatgtaaagtgtctaataaggccctgcccgcacctatcgaaaaaacaatctctaaggctaaagggcagccacgtgaaccacaagtatatactctgccaccttgccgcgatgagttgactaaaaaccaggtgagtttgtggtgtttggtaaaaggcttttacccttctgacatagccgtggaatgggagtccaacgggcaaccagagaacaattacaaaacaactcctcctgtcctggacagtgacggttctttttttctctattcaaagctgaccgtggacaagtcaagatggcaacagggcaacgtcttctcttgtagtgtgatgcacgaagccttgcataatcattatacccagaaatccctttcactctctcctggtaag

SEQ ID NO: 22 - protein, 212 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - DNA, 636 bp, synthetic construct
  source 1..636: mol_type = other DNA
gacatacagatgacacagagcccctcttccttgtctgcttctgtgggtgatcgcgtcacaattacctgccgcgctagtcaatccatatcaaacaacctgaactggtatcaacagaagcccggaaaggcccccaagctcctcatttactacacatccagattccactccggtgttccatctaggttcagcggctcagggtctggcacagacttcacattcacaatcagctcacttcagcccgaggacatagctacatactattgtcagcaggaacacacactcccttacacatttggccaaggaaccaaagtaactgtactttcatccgcaagcactaagggtccatcagtctttccacttgcaccttcatctaaatccacctcaggtggtactgcagcactcggttgtttggtaaaagattactttcctgagccagtgactgttagctggaatagtggagcccttacctccggtgtccatacattccccgctgtgttgcaatcatctggtctctattccctttcatctgtagttaccgtccctagctcctctctgggcacacaaacatatatttgtaacgtgaaccataaaccctccaacaccaaagttgataagaaggtcgagcccaaatcttgt

SEQ ID NO: 24 - protein, 581 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1743 bp, synthetic construct
  source 1..1743: mol_type = other DNA
gaagtacaattggtggaaagcggcggaggccttgtccaacctggcggttctttgcggctgtcctgtgccgctagtggttttacctttagtgatgcctggatggactgggtgcggcaggcccctggtaagggcctggaatgggtcgctgaaataagaaataaggcaaataaccatgcaagacattacgctgaaagtgtgaagggcaggttcactatatcccgagataacgcaaagaacagtttgtaccttcagatgaactccttgcgtgccgaggataccgccgtctactactgcgctcgcacatactactacggttcatcatacgggtactgcgacgtctgggggcaaggaacacttgtcaccgtttcttctgcctctactaagggaccccaggttcagttgcaggagtccgggcccggtctggtaaagccttctgaaaccctgagtttgacctgtaccgtgtccggtttctcactgattggctatgatctcaactggatacgacagccccccgggaagggcctggagtggattggcatcatctggggagatggaaccaccgactacaactccgctgtcaaaagtagagtgactatttccaaagacaccagcaagaaccaattctcactgaaattgagttcagttacagctgccgacactgctgtttactattgtgcaagagggggatattggtatgccacttcctattattttgactattggggccagggaacacttgtaacagtatcctcagcctcaacaaaaggaccatctgtatttccccttgcacccagttcaaagtctacttcaggtgggactgccgctctcggctgtctggtaaaagattatttccccgagccagttactgtaagttggaatagcggtgcactgacaagtggggtccatactttccctgccgtgcttcagagttccggcctttattcactctctagtgtagttaccgttccctcatctagccttggaacccaaacctatatctgtaatgtgaatcataaaccatctaatactaaggttgataaaaaggttgaacctaagagctgcgataaaacacatacctgtcctccttgtcctgctcctgaagctgctggtggaccttcagtgtttctgttcccccccaagccaaaagatacactgatgatctctcgtactcctgaggttacttgtgtcgtcgtcgatgtgtcacacgaagatcccgaagtgaagttcaactggtacgtcgatggtgttgaagtacacaatgccaaaacaaaacccagagaggagcaatacaatagtacataccgcgtagtgtctgttctcaccgtattgcatcaggactggctgaacggtaaagagtacaaatgtaaagtttctaataaagcactcccagcccctatagaaaagaccatcagcaaagctaaagggcagccccgtgagccacaagtctacacattgcctcctagtagggatgagctgacaaaaaatcaagtgtccctgacctgcctcgtgaaaggattttatccttctgatatagcagttgaatgggaatccaacggtcaacctgagaataattataagaccactcccccagtgctggattctgatggcagtttcttcttgtattctaaattgactgtcgacaaatcccgttggcaacagggaaatgtgttttcatgctctgttatgcacgaggctttgcacaatcactacactcagaagtccctgagcttgtctccaggaaaa

SEQ ID NO: 26 - protein, 327 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 981 bp, synthetic construct
  source 1..981: mol_type = other DNA
gacatacagatgacacagtctcctagctccctgtccgcaagcgttggagatagagtcacaataacttgtagaacatcagaaaacatatatagctacttggcttggtaccaacagaagcctggtaaggcacccaagctcctcatatacaacgccaagacccttgccgagggtgtgccaagcagattcagcgggtcagggagcgggaccgattttacacttactatttcctctttgcagcccgaagactttgctacttactactgtcagcaccactacggcactccttggactttcggacaaggtactaaacttgaaataaagaggaccgtagccgcccccgatatacagatgactcaaagtcctagttccctttccgctagcgtaggagaccgcgtaacaattacctgtagagcatcacaaagtataagcaacaatctgaactggtaccagcagaaacctgggaaggcccctaagttgctgatctattacacctccaggtttcatagtggcgtaccatctcgtttcagtggatcaggttctggcaccgatttcacttttaccatatcctcactccagcccgaagatatcgctacctactattgtcaacaggaacacacccttccttatacatttgggcaggggaccaagcttgagatcaaacggactgtggcagcacctagtgtcttcatattccccccttccgacgagcaactgaaaagtggtactgcttcagtagtttgcttgttgaacaacttctatccacgcgaagcaaaagtgcaatggaaggtcgataacgcacttcaatctggtaactctcaagaaagtgtcacagagcaggacagtaaggatagtacatatagccttagttcaacactgactctttctaaggccgattatgagaaacacaaggtatacgcttgtgaagtgacccaccagggactttctagcccagttaccaaatccttcaacagaggagagtgc

SEQ ID NO: 28 - protein, 581 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPEVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1743 bp, synthetic construct
  source 1..1743: mol_type = other DNA
caggtacagctccaggaatccgggcccggtcttgtgaaaccttcagaaacactgtctttgacttgtacagtgagtggattttctctcatcgggtacgatctcaactggatccggcaacctcctgggaaaggtctcgaatggatagggatcatctggggcgatggcactaccgattataattcagccgtgaagtctcgagtaacaatctccaaggataccagtaagaaccagtttagtctgaaactcagctctgttacagccgccgacacagctgtctactattgcgccaggggagggtattggtatgctaccagttattatttcgactattggggacagggaactttggtaaccgtcagtagtgcatctactaaaggccccgaggtccagcttgttgagtctgggggtggtctcgtccaacctgggggttccttgcggctgtcatgtgcagcttccggcttcacttttagtgatgcttggatggattgggtccggcaggccccaggtaagggtttggaatgggtggccgaaataagaaataaggccaacaatcatgctcgccactacgcagagtccgtaaaaggacgatttacaatcagccgtgacaacgcaaaaaatagcttgtatcttcaaatgaacagtttgagggctgaggatacagcagtgtattactgtgctcgaacttactactacgggagtagttatggttactgtgatgtatggggtcagggaactctggtaaccgtctcaagcgcttcaactaaaggtccttcagtattccccttggctccaagctccaagagtaccagtggcggtaccgcagctttgggctgcctggtaaaagattatttcccagaaccagtaactgtaagctggaatagcggggctttgacatcaggggtacacacctttccagctgtgcttcagtcttcaggcttgtacagtttgtcttccgtagtcacagtcccatcttctagtctggggacccaaacctatatctgtaatgtcaatcataagccctcaaatacaaaagtggacaaaaaagtagagcctaagagctgtgataaaacacatacatgccctccttgtcctgcccccgaagccgccggcggtccctcagtatttctttttccaccaaaacccaaagataccctcatgatcagtcgcaccccagaagtcacctgtgtcgtggtagatgtaagccacgaggatcccgaggtcaaattcaactggtatgtggatggcgtagaagttcacaacgccaagacaaagcccagggaagaacagtacaattcaacctacagggttgtctctgtactgaccgtccttcaccaagactggttgaacggtaaagagtataaatgtaaggtatccaacaaagctttgccagcacctatagagaaaaccatctccaaagctaagggtcaacccagagagccacaagtgtataccctgcctcccagtagagatgaactgacaaagaaccaagtttctctgacttgcttggtcaaagggttctatcctagcgacatcgctgtagaatgggaatcaaatggacaacctgaaaataactacaagactaccccacccgtgcttgacagtgatggcagttttttcctttacagtaagctcactgtcgacaagagccgatggcagcaaggcaacgtcttctcttgttccgttatgcatgaggccctccacaaccattatacccaaaagagtttgtcattgtcccctggtaaa

SEQ ID NO: 30 - protein, 327 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPDIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 981 bp, synthetic construct
  source 1..981: mol_type = other DNA
gacatccagatgacacagtcaccatcctctcttagtgcaagtgtgggcgatcgggttacaataacttgtagagcctctcaatcaatttcaaacaatctgaattggtatcaacagaagcctggcaaggcaccaaaacttttgatctactacacttcacgtttccattctggtgtaccatctcgcttctccggaagtggaagtggcacagatttcacatttaccattagttccttgcagcccgaagatatagccacttactattgtcagcaggaacataccctgccatacactttcggccagggcacaaaactcgagatcaagcgtactgtggcagcccctgatattcagatgacccagtccccaagttcactctcagcttctgtcggcgaccgtgtaacaataacctgccgtacctctgaaaacatatactcatacttggcctggtatcaacaaaaaccaggtaaagctcctaagttgctcatctataatgctaagactttggctgaaggagtgccctctcgattctctgggtcaggatccggaacagatttcacactgactatatccagcttgcaaccagaagacttcgccacttattactgccaacatcactatgggacaccctggacatttgggcaaggaaccaaactggagataaagcggactgtggccgcaccttccgttttcatctttccaccctctgacgaacaactcaaatcagggactgcttcagtcgtgtgtctcctcaataatttttatcctcgggaggctaaagtccaatggaaagtagacaatgccctccaatccgggaatagtcaggaaagcgtcacagagcaagattcaaaagacagtacctactcattgtctagtacccttacccttagtaaagcagactacgagaaacataaggtttatgcctgcgaggtgactcaccagggtttgtctagcccagtgactaaatccttcaatcgcggggagtgt

SEQ ID NO: 32 - protein, 712 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSGGGGSGGGGSGGGGSDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIK

SEQ ID NO: 33 - DNA, 2142 bp, synthetic construct
  source 1..2142: mol_type = other DNA
gaggtgcagctggtggaaagcggcggggggctggtgcagcccggaggctcactgagactgtcttgcgctgccagcggcttcaccttcagcgacgcctggatggactgggtgaggcaggcccctgggaaggggctggagtgggtggctgagatccggaataaggccaataaccacgccaggcactatgccgagagtgtgaaaggcagatttaccatctctagggacaatgccaagaactcactgtacctgcagatgaactccctgagagccgaggacaccgccgtgtactactgtgccagaacctattactacggaagcagctacggctattgcgatgtgtggggacagggaaccctggtgactgtgtcttcctgagcctccaccaagggcccctccgtgttcccactggccccgagtagcaagtctacctccggcgggacagccgcactgggctgcctggtgaaggactactttcccgagcctgtgacagtgagctggaattccggggccctgacatcaggcgtgcacaccttcccagccgtgctccagagctctggcctgtatagtctgagcagtgtggtgaccgtgccctccagctccctgggaactcagacctatatctgtaacgtgaaccacaagccttctaataccaaggtggataagaaagtggaaccaaagtcatgcgacaaaacacacacctgccccccctgccccgcccctgaagccgccggcgggcctagcgtgtttctgttccctcctaagcctaaggacacactgatgatttccaggacccccgaggtgacctgcgtcgtcgtggacgtgagccacgaggaccccgaggtgaaattcaactggtacgtggacggcgtggaagtgcacaacgctaaaaccaagcctagagaggaacagtacaactctacatacagggtggtgtccgtcttgactgtgctgcaccaggactggctgaacggcaaggaatataagtgcaaggtgagcaataaagcactccccgcccccattgaaaaaaccatcagcaaggctaagggccagcctagggaaccacaggtgtataccctgccacctagcagagacgagctgaccaagaatcaggtgtctctgacatgtctggtgaaggggttttaccctagcgacattgccgtcgagtgggagtctaatggccagcctgagaacaattataagactacccctcctgtgctggacagtgatggctcattttttctgtactctaagctgacagtggacaagagcaggtggcagcagggcaatgtgttcagctgcagcgtgatgcacgaggccctgcataatcattatacccagaagtctctgtctctcagcccaggcaaaggcggaggggggtctggcggcgggggttccggcggcgggggctcccaggtgcagctgcaggagagcggacctggcctggtgaaacccagcgagaccctgtccctgacatgcaccgtgagcggcttctccctgatcgggtatgacctgaattggatccggcagcctcctggcaagggtctggagtggatcggaatcatctggggcgacggcaccaccgattataacagcgccgtgaagtctcgggtgactatctctaaggatacttccaagaaccagttttccctgaagctgagctctgtgaccgctgccgataccgccgtgtactactgcgccaggggcggttactggtacgccaccagctactacttcgactactggggccagggcaccctggtgaccgtgtccagcggcggcggcggctctggcggtgggggctccggcggcggagggtctgacattcagatgacccagagcccctcctccctgtctgcctccgtgggcgacagagtgaccatcacatgtagagcctcccagtcaatatctaacaacctgaattggtatcagcagaagcccggcaaagcccctaagctgctgatttactacacttctcgctttcactccggagtgcccagcagatttagtgggagcggctctgggacagacttcacttttaccatcagtagcctgcagcccgaggacatcgccacctattactgccagcaggagcataccctgccctacactttcggacaggggaccaagctggagatcaagtga

SEQ ID NO: 34 - protein, 572 aa, synthetic construct
EVQLVESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 327 aa, synthetic construct
DIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRTVAAPDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 578 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPEVQLVESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 327 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPDIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 444 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39 - protein, 214 aa, synthetic construct; identical to SEQ ID NO: 4

SEQ ID NO: 40 - protein, 702 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSGGGGSGGGGSGGGGSDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIK

SEQ ID NO: 41 - protein, 588 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 334 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 588 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPEVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 334 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPDIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 579 aa, synthetic construct
EVQLVESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPSVFPLAPQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 334 aa, synthetic construct
DIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRTVAAPSVFIFPPDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 585 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPEVQLVESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 334 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPDIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 445 aa, synthetic construct
EVQLLESGGGLVQPGGSLRLSCAASGFTFSSYWMHWVRQAPGKGLEWVAYINPSTGYTEYNQKFKRFTISRDSKNTVYLQMNSLRAEDTAVYYCTSGGYDDLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 460 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNDIEGRMDPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 614 aa, synthetic construct
AAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEIEGRIDPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 454 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFLLYSKLTVDKSRWQQGNVFSCSVMHEALHNHYTQKSLSLSPGK

SEQ ID NO: 53 - protein, 451 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 54 - protein, 214 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 444 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFLLYSKLTVDKSRWQQGNVFSCSVMHEALHNHYTQKSLSLSPGK

SEQ ID NO: 56 - protein, 452 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 212 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 58 - protein, 578 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 327 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 568 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 326 aa, synthetic construct
DIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 578 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPEVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 327 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPDIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 568 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPEVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 326 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPDIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 585 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 334 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 575 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 333 aa, synthetic construct
DIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 585 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPEVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 334 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPDIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 575 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPEVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 333 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPDIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 573 aa, synthetic construct
EVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 332 aa, synthetic construct
DIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 570 aa, synthetic construct
EVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 331 aa, synthetic construct
DIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPDIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 575 aa, synthetic construct
EVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 332 aa, synthetic construct
DIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 572 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPEVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 81 - protein, 331 aa, synthetic construct
DIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPDIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 577 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPEVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 83 - protein, 332 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPDIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 577 aa, synthetic construct; identical to SEQ ID NO: 82

SEQ ID NO: 85 - protein, 332 aa, synthetic construct; identical to SEQ ID NO: 83

SEQ ID NO: 86 - protein, 580 aa, synthetic construct
EVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPSVFPLAPQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 339 aa, synthetic construct
DIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 577 aa, synthetic construct
EVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPSVFPLAPQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 338 aa, synthetic construct
DIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPDIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 582 aa, synthetic construct
EVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPSVFPLAPEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 339 aa, synthetic construct
DIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 586 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPEVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 339 aa, synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPDIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 579 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPEVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 95 - protein, 338 aa, synthetic construct
DIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPDIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 584 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPEVQLVESGGGLVQPGGSLRLSCAASGFTFSSYWMHWVRQAPGKGLEWVAYINPSTGYTEYNQKFKRFTISRDDSKNTVYLQMNSLRAEDTAVYYCTSGGYDDLGYWGQGTLVTVSSASTKGPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 97 - protein, 339 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPDIQMTQSPSSLSASVGDRVTITCRSSKSLLYKDGKTYLYWYQQKPGKAPKLLIYLMSTRASGVPSRFSGSGSGTDFTLTISSLQPEDFATYYCQQLVUYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 448 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 99 - protein, 214 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 454 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFFLYSRLTVDKSRWQQGNVFSCSVMHEALHNHYTQKSLSLSPGK

SEQ ID NO: 101 - protein, 448 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFKLYSKLTVDKSRWQQGNVFSCSVMHEALHNHYTQKSLSLSPGK

SEQ ID NO: 102 - protein, 444 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFFLYSRLTVDKSRWQQGNVFSCSVMHEALHNHYTQKSLSLSPGK

SEQ ID NO: 103 - protein, 457 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 212 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 105 - protein, 583 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 327 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 573 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 326 aa, synthetic construct
DIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 583 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPEVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 327 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPDIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 573 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPEVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 326 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPDIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 590 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 334 aa, synthetic construct
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 580 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 333 aa, synthetic construct
DIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 590 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPEVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 334 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPDIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 580 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPEVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 333 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPDIQMTQSPSSLSASVGDRVTITCKASQSVGTTIVWTQQLPGLAPKLLIYSASNRHTGVPSRFSGSGSGTDFTLTISSLQPEDFATYYCQQYTSYPFTFGQGTKEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 453 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 122 - protein, 214 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 453 aa, synthetic construct
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 124 - protein, 451 aa, synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 125 - protein, 451 aa, synthetic construct; identical to SEQ ID NO: 124

SEQ ID NO: 126 - protein, 451 aa, synthetic construct
EVQLLESGGGLVQPGGSLRLSCAASGFTFSGRTVSSYAMGWVRQAPGKGLEWVAGISRSAERTYRFTISRDSKNTVYLQMNSLRAEDTAVYYCTSDLDPNRIFSREEYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 127 - protein, 445 aa, synthetic construct
EVQLLESGGGLVQPGGSLRLSCAASGFTFSSYWMHWVRQAPGKGLEWVAYINPSTGYTEYNQKFKRFTISRDSKNTVYLQMNSLRAEDTAVYYCTSGGYDDLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFLLYSKLTVDKSRWQQGNVFSCSVMHEALHNHYTQKSLSLSPGK

SEQ ID NO: 128 - protein, 219 aa, synthetic construct
DIQMTQSPSSLSASVGDRVTITCRSSKSLLYKDGKTYLYWTQQLPGLAPKLLIYLMSTRASGVPSRFSGSGSGTDFTLTISSLQPEDFATYYCQQLVUYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 445 aa, synthetic construct
EVQLLESGGGLVQPGGSLRLSCAASGFTFSSYWMHWVRQAPGKGLEWVAYINPSTGYTEYNQKFKRFTISRDSKNTVYLQMNSLRAEDTAVYYCTSGGYDDLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLVKGFYPSDIAVEWESNGQPENNYKTTPPVLDSDGSFFLYSRLTVDKSRWQQGNVFSCSVMHEALHNHYTQKSLSLSPGK

SEQ ID NO: 130 - protein, 714 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIK

SEQ ID NO: 131 - protein, 709 aa, synthetic construct
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSGGGGSGGGGSGGGGSDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIK

SEQ ID NO: 132 - protein, 704 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIK

SEQ ID NO: 133 - protein, 699 aa, synthetic construct
EVQLLESGGGLVQPGGSLRLSCAASGFTFSDAWMDWVRQAPGKGLEWVAEIRHKANDHAIFYDESVKGRFTISRDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSGGGGSGGGGSGGGGSDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIK

SEQ ID NO: 134 - protein, 709 aa, synthetic construct
EVQLLESGGGLVQPGGSLRLSCAASGFTFSGRTVSSYAMGWVRQAPGKGLEWVAGISRSAERTYRFTISRDSKNTVYLQMNSLRAEDTAVYYCTSDLDPNRIFSREEYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSGGGGSGGGGSGGGGSDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIK

SEQ ID NO: 135 - protein, 711 aa, synthetic construct
EVQLLESGGGLVQPGGSLRLSCAASGFTFSGRTVSSYAMGWVRQAPGKGLEWVAGISRSAERTYRFTISRDSKNTVYLQMNSLRAEDTAVYYCTSDLDPNRIFSREEYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIK

SEQ ID NO: 136 - protein, 706 aa, synthetic construct
EVQLLESGGGLVQPGGSLRLSCAASGFTFSGRTVSSYAMGWVRQAPGKGLEWVAGISRSAERTYRFTISRDSKNTVYLQMNSLRAEDTAVYYCTSDLDPNRIFSREEYAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSGGGGSGGGGSGGGGSDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIK

SEQ ID NO: 137 - protein, 703 aa, synthetic construct
EVQLLESGGGLVQPGGSLRLSCAASGFTFSSYWMHWVRQAPGKGLEWVAYINPSTGYTEYNQKFKRFTISRDSKNTVYLQMNSLRAEDTAVYYCTSGGYDDLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSGGGGSGGGGSGGGGSDIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIK

SEQ ID NO: 138 - protein, 705 aa, synthetic construct
EVQLLESGGGLVQPGGSLRLSCAASGFTFSSYWMHWVRQAPGKGLEWVAYINPSTGYTEYNQKFKRFTISRDSKNTVYLQMNSLRAEDTAVYYCTSGGYDDLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKLEIK

SEQ ID NO: 139 - protein, 700 aa, synthetic construct
EVQLLESGGGLVQPGGSLRLSCAASGFTFSSYWMHWVRQAPGKGLEWVAYINPSTGYTEYNQKFKRFTISRDSKNTVYLQMNSLRAEDTAVYYCTSGGYDDLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIGVVTNFDNWGQGTLVTVSSGGGGSGGGGSGGGGSDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKLEIK

SEQ ID NO: 140 - protein, 930 aa, Homo sapiens
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 141 - protein, 930 aa, Macaca fascicularis
MLLGWASLLLCAFRLPLAAAGPAAAPAQDKAGQPATAAAAAQPSRRQGEEVQERTEPPGHPHPLAQRRSSKGLVQNIDQLYSGGGKVGYLVYAGGRRFLLDLERDGSVGTAGFVPTGGGTSAPWRHRSHCFYRGTVDGSPRSLAVFDLCGGLDGFFAVKHARYTLKPLLRGPWAEEETGRVYGDGSARILHVYTREGFSFEALQPRASCETPASTPEPHERPPAHSNPGGRAALASQLLDQSAVSPAGGPGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDQPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCGLMPCPPNGKSFRHEQCEAKNGYQSDAKGVKTFVEWVPKYAGVLPADVCKLTCRAKGTGYYVVFSPKVTDGTECRPYSNSVCVRGKCVRTGCDSIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQLQWVTGPWLACSRTCDTGWHTRTVQCQDGNRKLAKGCPLSQRPSAFKQCLLKKC

SEQ ID NO: 142 - protein, 930 aa, Felis sp.
MLLGWASLLLCALRLPPVAAGPAAAPAQDTAGQPRAAAAAAQPRGRQGEEAQERAEPPGHPHPLAPQRRSGGLVHNIDQIYAGGGKVGYLVYAGGRRFLLDLERDGSLGAAGFAPAGSGPGASRRHRDHCFHRGTVDGSPRSLAVFDLCGGLDGFFAVKHARYTLKPLLRGPRAEAEAGRVYGDGSSRVLHVYTREGFSFEAVPPRASCETPASPPGPRERPPAHGGPGPRWELAPPFPDQTVPSSEGTQGPQTWWRRRRRSISRARQVELLLVADASMARMYGRGLQHYLLTLASIANRLYSHASIENHIRLAVVKVVVLGDKDKSLEVSKNAATTLKNFCKWQHQHNQLEDDHEEHHDAAILFTREDLCGHHSCDTLGMADVGTICSPERSCAVIEDDGLHAAFTVAHEIGHLLGLSHDDSKFCEENFGSTEDKRLMSSILTSIDASKPWSKCTSATITEFLDDGHGNCLLDVPRQQISGPEELPGQTYDATQQCNLTFGPEYSVCPGMDVCTRLWCAVVRQGQMVCLTKKLPAVEGTPCGKGRICLQGKCVDKTKKKYYSTSSHGNWGSWGPWGQCSRSCGGGVQFAYRHCNNPAPRNSGRYCTGKRAIYRSCSVTPCPPNGKSFRHEQCEAKNGYQSDAKGVKTFVEWVPKYAGVLLGDVCKLTCRAKGTGYYVVFSPKVTDGTECRPYSNSVCVRGKCVRTGCDGIIGSRLQYDKCAVCGGDNSSCTKVVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKALDVRYSFFVPKKSTQKVNSVSSHGSNKVGSHTPQLQWVTGPWLACSRTCDTGWHTRTVQCQDANRKLAKGCLLSQRPSAFKQCLLKKC

SEQ ID NO: 143 - protein, 934 aa, Canis sp.
MLLGWASLLLGALRLPPVAAGPAAAPAQDKAGQPWAAAAAAQPRRRQGEEAREPAEPPGHPHPLAPQRRSSGLVQNVDQIYAGGGKVGYLVYAGGRRFLLDLERDGSVGAAGSAPAGRGPGAPRRHRDHCFYRGTVDGSPRSLAVLDLCGGLDGFFAVRHARYTLKPLLRGPWAGAGAGAEAERVYGDGSPRILHVYTREGFSFEALPPRTSCETPASPPGPRERPPAHSSPDPRWSPAPPFPAPPAASPDGGPGPQTWWRRRRRSISRARQVELLLVADASMARMYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCNVTPCPPNGKSFRHEQCEAKNGYQSDAKGVKTFVEWVPKYAGVLLGDVCKLTCRAKGTGYYVVFSPKVTDGTECRPYSNSVCVRGKCVRTGCDGIIGSKLQYDKCAVCGGDNSSCTKVVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKALDVRYSFFVPKKSTQKVNSVTSHASNKVGSHTPQLQWVTGPWLACSRTCDTGWHTRTVQCQDANRKLAKGCLLSQRPSAFKQCLLKKC

SEQ ID NO: 144 - protein, 929 aa, Equus caballus
MLLGWASLLLCALRLPLVAAGPAAAPAQDKTGQPRAAAAAAQPRRRQGEEAQERAEPPGHPHPLAPQRRSSGLVQNIDQIYSGGGKVGYLVYAGGRRFLLDLERDGSVGAAGFVPVGGGPSATRRHRGHCFYRGTVDGSPRSLAVFDLCGGLDGFFAVKHARYTLKPLLRGPWAETETGRVYGDGSARILHVYTREGFSFEALPPRTSCETPASPPGSRERPPAHSSPEPRWALAPQFPDQSATSSDGGQGSQTWRRRRRSISRARQVELLLVADASMARMYGRGLQHYLLTLASIANRLYSHASIENHIRLAVVKMVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGSTEEKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSVTPCPANGKSFRHEQCEAKNGYQSDAKGVKTFVEWVPKYAGVLPGDVCKLTCRAKGTGYYVVFSPKVTDGTECRPYSNSVCVRGKCVRTGCDGIIGSKLQYDKCGVCGGDNSSCTKVVGTFNKKSKGYTDVVRIPEGATHIKVRQFKTKDQTRFTAYLALKKKNGEYLINGKYMISTSETIIDINGTVMNYSGWSHRDDFLHGMGYSATKEILIVQILATDPTKALDVRYSFFVPKKPTQKGNSVTSHGSNKVGSTTPQLQWVTGPWLACSRTCDTGWHTRTVQCQDGNRKLAKGCLLSQRPSAFKQCLLKKC

SEQ ID NO: 145 - protein, 930 aa, Mus musculus
MRLEWAPLLLLLLLLSASCLSLAADSPAAAPAQDKTRQPQAAAAAAEPDQPQGEETRERGHLQPLAGQRRSGGLVQNIDQLYSGGGKVGYLVYAGGRRFLLDLERDDTVGAAGSIVTAGGGLSASSGHRGHCFYRGTVDGSPRSLAVFDLCGGLDGFFAVKHARYTLKPLLRGSWAEYERIYGDGSSRILHVYNREGFSFEALPPRASCETPASPSGPQESPSVHSRSRRRSALAPQLLDHSAFSPSGNAGPQTWWRRRRRSISRARQVELLLVADSSMARMYGRGLQHYLLTLASIANRLYSHASIENHIRLAVVKVVVLTDKDTSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGTTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRVCLQGKCVDKTKKKYYSTSSHGNWGSWGPWGQCSRSCGGGVQFAYRHCNNPAPRNSGRYCTGKRAIYRSCSVTPCPPNGKSFRHEQCEAKNGYQSDAKGVKTFVEWVPKYAGVLPADVCKLTCRAKGTGYYVVFSPKVTDGTECRPYSNSVCVRGRCVRTGCDGIIGSKLQYDKCGVCGGDNSSCTKIIGTFNKKSKGYTDVVRIPEGATHIKVRQFKAKDQTRFTAYLALKKKTGEYLINGKYMISTSETIIDINGTVMNYSGWSHRDDFLHGMGYSATKEILIVQILATDPTKALDVRYSFFVPKKTTQKVNSVISHGSNKVGPHSTQLQWVTGPWLACSRTCDTGWHTRTVQCQDGNRKLAKGCLLSQRPSAFKQCLLKKC

SEQ ID NO: 146 - protein, 927 aa, Rattus sp.
MRLEWASLLLLLLLLCASCLALAADNPAAAPAQDKTRQPRAAAAAAQPDQRQWEETQERGHPQPLARQRRSSGLVQNIDQLYSGGGKVGYLVYAGGRRFLLDLERDDTVGAAGGIVTAGGLSASSGHRGHCFYRGTVDGSPRSLAVFDLCGGLDGFFAVKHARYTLKPLLRGSWAESERVYGDGSSRILHVYTREGFSFEALPPRTSCETPASPSGAQESPSVHSSSRRRTELAPQLLDHSAFSPAGNAGPQTWWRRRRRSISRARQVELLLVADSSMAKMYGRGLQHYLLTLASIANRLYSHASIENHIRLAVVKVVVLTDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGSTEDKRLMSSILTSIDASKPWSKCTSATITEFLDDGHGNCLLDVPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGPWGQCSRSCGGGVQFAYRHCNNPAPRNSGRYCTGKRAIYRSCSVIPCPPNGKSFRHEQCEAKNGYQSDAKGVKTFVEWVPKYAGVLPADVCKLTCRAKGTGYYVVFSPKVTDGTECRPYSNSVCVRGRCVRTGCDGIIGSKLQYDKCGVCGGDNSSCTKIIGTFNKKSKGYTDVVRIPEGATHIKVRQFKAKDQTRFTAYLALKKKTGEYLINGKYMISTSETIIDINGTVMNYSGWSHRDDFLHGMGYSATKEILIVQILATDPTKALDVRYSFFVPKKTTQKVNSVISHSSNKVGLHSPQLQWVTGPWLACSRTCDTGWHTRTVQCQDGNRKLAKGCILSQRPSAFKQCLLKKC

SEQ ID NO: 147 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 148 - protein, 241 aa, Macaca fascicularis
MSMLFYTLITAFLIGIQAEPHSESNVPAGHTIPQAHWTKLQHSLDTALRRVRSAPAVAIAARVAGQTRNITVDPRLFKKRRLRSPRVLFSTQPPPEAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 149 - protein, 241 aa, Canis sp.
MSMLFYTLITALLIGIRAEPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 150 - protein, 241 aa, Felis sp.
MSMLSYTLITALLIGIQAEPHPESNVPAGHTIPQAHWTKLQHSLDTALRRARSTPAGAIAARVAGQTRNITVDPKLFKKRRLRSPRVLFSTHPPPVAADTQGLDLEAGGAASFNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 151 - protein, 241 aa, Equus caballus
MSMLFYTLITALLIGTQAEPHTESNVPAGHAIPQAHWTKLQHSLDTALRRARSAPARAIAARVAGQTRNITVDPKLFKKRRLRSPRVLFSTQPPPVAADTQDLDFEAGGAASFNRTHRSKRSSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKTGRKA

SEQ ID NO: 152 - protein, 241 aa, Mus musculus
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRG

SEQ ID NO: 153 - protein, 241 aa, Rattus sp.
MSMLFYTLITAFLIGVQAEPYTDSNVPEGDSVPEAHWTKLQHSLDTALRRARSAPAEPIAARVTGQTRNITVDPKLFKKRRLRSPRVLFSTQPPPTSSDTLDLDFQAHGTISFNRTHRSKRSSTHPVFHMGEFSVCDSVSVWVGDKTTATDIKGKEVTVLGEVNINNSVFKQYFFETKCRAPNPVESGCRGIDSKHWNSYCTTTHTFVKALTTDDKQAAWRFIRIDTACVCVLSRKAARRG

SEQ ID NO: 154 - protein, 10 aa, synthetic construct
GISRSAERTY

SEQ ID NO: 155 - protein, 15 aa, synthetic construct
DLDPNRIFSREEYAY

SEQ ID NO: 156 - protein, 16 aa, synthetic construct
YINPSTGYTEYNQKFK

SEQ ID NO: 157 - protein, 8 aa, synthetic construct
GGYDDLGY

SEQ ID NO: 158 - protein, 7 aa, synthetic construct
LMSTRAS

SEQ ID NO: 159 - protein, 9 aa, synthetic construct
QQLVUYPYT